(19) United States
(12) Patent Application Publication
(10) Pub. No.: US 2026/0083740 A1
(43) Pub. Date: Mar. 26, 2026

(54) USE OF TAVAPADON IN TREATING PARKINSON'S DISEASE

(71) Applicant: Cerevel Therapeutics, LLC, Boston, MA (US)

(72) Inventor: Sridhar Duvvuri, Lexington, MA (US)

(21) Appl. No.: 19/337,808

(22) Filed: Sep. 23, 2025

Related U.S. Application Data
(60) Provisional application No. 63/699,137, filed on Sep. 25, 2024.
(60) Provisional application No. 63/727,555, filed on Dec. 3, 2024.
(60) Provisional application No. 63/790,534, filed on Apr. 17, 2025.

(51) Int. Cl. A61K 31/513; A61K 31/198; A61K 45/06; A61P 25/16

(52) U.S. Cl. CPC A61K 31/513; A61K 31/198; A61K 45/06; A61P 25/16

(57) ABSTRACT

The present disclosure relates to methods for treating a patient diagnosed with Parkinson's Disease (PD) by administering an escalating dose of tavapadon and monitoring efficacy of PD treatment.

### 1. RELATED APPLICATIONS

This application claims the benefit of U.S. Provisional Application Nos. 63/790,534, filed Apr. 17, 2025, 63/727,555, filed Dec. 3, 2024, and 63/699,137, filed Sep. 25, 2024, the disclosures of each of which are incorporated by reference in their entireties.

### 2. FIELD

Provided herein are methods for treating Parkinson's Disease (PD) in a patient comprising administering tavapadon in escalating doses to the patient.

### 3. BACKGROUND

Parkinson's disease (PD) is a progressive neurodegenerative disease characterized by deficits in motor control arising from the loss of dopaminergic neurons in the substantia nigra pars compacta and subsequent depletion of dopamine in motor regions of the striatum. Poewe et al., Nat Rev Dis Primers 2017; 3(1): 17013. Standard treatment remains the restoration of dopaminergic function through administration of the synthetic dopamine precursor levodopa (1-dopa) and subsequent activation of D1-like and D2-like dopamine receptors. Rizek et al., CMAJ 2016; 188: 1157-1165; Charvin et al., Nat Rev Drug Discov 2018; 17: 804-822; and Ossig C et al., Neurol Clin 2015; 33: 19-37. However, while use of levodopa provides efficacy and is generally well tolerated, long-term treatment can lead to fluctuating motor responses and dyskinesias in many individuals. Bastide et al., Prog Neurobiol 2015; 132: 96-168.

There has long been interest in the potential of D1- or D2-selective receptor agonists as an alternative to levodopa in subjects with Parkinson's disease. Clinical trials of limited duration with prototype full D1 agonists demonstrated that, while these agents exhibited robust anti-parkinsonian effects on motor symptoms challenges with pharmacokinetics, cardiovascular parameters, and side effects including dyskinesias made them ill-suited for further clinical advancement. Rascol et al., Ann Neurol 1999; 45:736-741. In contrast, efforts to develop D2-selective agonists with suitable clinical properties were more successful, with several receiving approval for the treatment of Parkinson's disease, including pramipexole, ropinirole, and rotigotine. In general, these agents exhibit less-frequent motor complications than levodopa, but are not as effective for controlling motor symptoms. Dietrichs et al., Acta Neurol Scand 2017; 136: 378-385. D2-selective agonists are also associated with an increased incidence of specific adverse effects (AEs), including nausea, somnolence, hypotension, compulsive behaviors, and hallucinations, particularly in the elderly. Stowe et al., Cochrane Database Syst Rev 2008: 2: CD006564. Thus, there remains a need for agents that provide effective and predictable control of motor symptoms while avoiding side effects associated with activation of D2-like receptors.

Existing clinical and preclinical data provide theoretical support for the long-standing hypothesis that selective activation of D1-like receptors may be an important therapeutic option for Parkinson's disease. Kim et al., Front Biol (Beijing) 2015; 10: 230-238. To date, however, attempts to develop D1-selective agonists have been hampered by tolerability issues (notably, acute hypotension) and poor pharmacokinetics (e.g. short half-life and low oral availability).

Tavapadon is a D1/D5 dopamine receptor agonist being investigated for the treatment of Parkinson's disease. By targeting the D1 dopamine receptor, tavapadon aims to improve motor control symptoms while minimizing adverse events (AEs) that may be mechanistically linked to D2/D3 dopamine receptor agonists, such as L-Dopa. There is continuing need to develop dosing schedules for tavapadon for more effective treatment of Parkinson's disease to provide sustained relief of motor symptoms with a reduced likelihood of adverse events.

### 4. SUMMARY

Provided herein are methods for the treatment of a patient suffering from Parkinson's disease, comprising administering tavapadon in an escalating dose schedule to the patient.

In certain embodiments, provided herein are methods for treating Parkinson's Disease in a patient, the methods comprising orally administering tavapadon to the patient. In one embodiment, the Parkinson's Disease is an early Parkinson's Disease. In one embodiment, the Parkinson's Disease is an advanced Parkinson's Disease. The criteria for early and advanced Parkinson's Disease are known to one of skill in the art and described elsewhere herein. An exemplary criteria are based on the Hoehn and Yahr scale.

In one embodiment, provided herein is a method for treating Parkinson's Disease in a patient, wherein the method comprises: a titration phase comprising orally administering tavapadon in escalating doses to the patient, an adjustment phase following the titration phase, wherein the dose is adjusted based on patient tolerability, and a maintenance phase wherein the patient is administered the highest tolerated dose achieved during the adjustment phase.

In one embodiment, the method provided herein comprises:
a titration phase comprising orally administering to the patient a daily dose of tavapadon in eight sequential dose escalation steps 1-8, wherein
   step 2 comprises administering to the patient a dose of 0.5 mg per day of tavapadon on days 5-8 of treatment,
   step 6 comprises administering to the patient a dose of 2.25 mg per day of tavapadon on days 21-24 of treatment,
   step 8 comprises administering to the patient a dose of a therapeutically effective amount of 5 mg per day of tavapadon,
   thereby treating the Parkinson's Disease.

In one embodiment of the method, the titration phase further comprises maintaining a dose of 3 mg per day of tavapadon once daily for a period of 7 or more days before increasing the dose of tavapadon.

In one embodiment of the method, the titration phase further comprises maintaining the 3 mg of tavapadon once daily dose for at least 15 days before increasing the dose of tavapadon.

In one embodiment of the method, the titration phase further comprises not exceeding the dose of 3 mg of tavapadon once daily until at least 40 days from the commencement of the titration phase.

In one embodiment, the method further comprises an adjustment phase following the titration phase, wherein the adjustment phase comprises:
step 9 comprising administering to the patient a dose of 10 mg per day dose of tavapadon, and
step 10 comprising administering to the patient a therapeutically effective amount of 15 mg per day dose of tavapadon;
wherein the dose of tavapadon is adjusted over the range of a therapeutically effective amount of 5, 10 or 15 mg per day of tavapadon in step 9 and step 10 based on patient tolerability.

In one embodiment, the method further comprises a maintenance phase following the adjustment phase, wherein the maintenance phase comprises administering to the patient a therapeutically effective amount of 5, 10 or 15 mg per day dose of tavapadon, wherein the patient is administered the highest tolerated dose achieved during the adjustment phase.

In one embodiment, the method comprises administering tavapadon to the patient in a dose of 3 or fewer oral pills.

In one embodiment, the method comprises administering to the patient a therapeutically effective amount of 5 mg per day of tavapadon during the maintenance phase.

In one embodiment, the method provided herein comprises administering to the patient a therapeutically effective amount of 15 mg per day of tavapadon during the maintenance phase.

In certain embodiments, the methods provided herein comprise:
a titration phase comprising orally administering to the patient:
0.25 mg per day dose of tavapadon for days 1-4 of the titration phase;
0.5 mg per day dose of tavapadon for days 5-8 of the titration phase;
0.75 mg per day dose of tavapadon for days 9-12 of the titration phase;
1.0 mg per day dose of tavapadon for days 13-16 of the titration phase;
1.5 mg per day dose of tavapadon for days 17-20 of the titration phase;
2.25 mg per day dose of tavapadon for days 21-24 of the titration phase;
3 mg per day dose of tavapadon for days 25-40 of the titration phase; and
a therapeutically effective amount of 5 mg per day dose of tavapadon for days 41-56 of the titration phase;
thereby treating the Parkinson's Disease.

In certain embodiments, the methods provided herein comprise:
i. a titration phase comprising orally administering to the patient:
   0.25 mg per day dose of tavapadon for days 1-4 of the titration phase;
   0.5 mg per day dose of tavapadon for days 5-8 of the titration phase;
   0.75 mg per day dose of tavapadon for days 9-12 of the titration phase;
   1.0 mg per day dose of tavapadon for days 13-16 of the titration phase;
   1.5 mg per day dose of tavapadon for days 17-20 of the titration phase;
   2.25 mg per day dose of tavapadon for days 21-24 of the titration phase;
   3 mg per day dose of tavapadon for days 25-40 of the titration phase; and a therapeutically effective amount of 5 mg per day dose of tavapadon for days 41-56 of the titration phase;
ii. followed by an adjustment phase comprising orally administering to the patient:
   a therapeutically effective amount of 10 mg per day dose of tavapadon, and
   a therapeutically effective amount of 15 mg per day dose of tavapadon,
   wherein the dose of tavapadon is adjusted over the range of 5, 10 or 15 mg based on patient tolerability; and
iii. followed by a maintenance phase comprising orally administering to the patient:
   a therapeutically effective amount of 5, 10 or 15 mg per day dose of tavapadon;
   wherein the patient is administered the highest tolerated dose achieved during the adjustment phase and the maintenance phase maintains the patient on a steady dose of tavapadon,
   thereby treating the Parkinson's Disease.

In one embodiment, the methods provided herein comprise administering to the patient a therapeutically effective amount of 5 mg per day of tavapadon.

In one embodiment, the methods provided herein comprise administering to the patient a therapeutically effective amount of 10 mg per day of tavapadon.

In one embodiment, the methods provided herein comprise administering to the patient a therapeutically effective amount of 15 mg per day of tavapadon.

In one embodiment, the methods provided herein comprise administering to the patient a safe and effective treatment of Parkinson's Disease, wherein the method comprises:
a titration phase comprising orally administering escalating tavapadon doses to the patient, wherein the dose of tavapadon does not exceed a therapeutically effective amount of 5 mg per day until after at least 40 days from the commencement of the titration phase, and
a maintenance phase after the titration phase, comprising orally administering once daily to the patient a therapeutically effective amount of a maintenance dose of tavapadon.

In certain embodiments, provided herein a method for treating Parkinson's Disease in a patient, wherein the method comprises a titration phase comprising orally administering to the patient a daily dose of tavapadon in dose escalation steps, wherein the dose of tavapadon does not exceed a therapeutically effective amount of 5 mg per day until after at least day 56 of the titration phase, thereby treating the Parkinson's Disease.

In certain embodiments, the patient is about 40 to 80 years of age.

In certain embodiments, the patient is not previously treated with a medication for Parkinson's Disease.

In certain embodiments of the methods provided herein, the patient is previously or concurrently treated with a therapeutically effective dose of one or more other medications for Parkinson's Disease. In certain embodiments of the methods provided herein, the patient is previously and concurrently treated with a therapeutically effective dose of one or more other medications for Parkinson's Disease.

In certain embodiments of the methods provided herein, the patient is previously and/or concurrently treated with a therapeutically effective dose of dopaminergic agent. In certain embodiments of the methods provided herein, the patient is previously and/or concurrently treated with a therapeutically effective dose of L-Dopa. In certain embodiments of the methods provided herein, the patient is previously and/or concurrently treated with a therapeutically effective dose of one or more COMT inhibitors, MAO-B inhibitors, amantadine, istradefylline, and anticholinergic drugs. In certain embodiments, patient is previously or concurrently treated with a therapeutically effective dose of monoamine oxidase B inhibitor.

In certain embodiments, the methods provided herein comprise administering L-Dopa concurrently with or prior to the treatment with tavapadon. In certain embodiments, the methods provided herein comprise administering L-Dopa concurrently with and prior to the treatment with tavapadon. In one embodiment, the methods provided herein comprise administering a stable dose of L-Dopa at least 4 weeks prior to and concurrently with the treatment with tavapadon. In one embodiment, the methods provided herein comprise administering a minimum total daily dose of 400 mg of L-Dopa at least 4 weeks prior to and concurrently with the treatment with tavapadon. In one embodiment, the methods provided herein comprise administering a minimum total daily dose of 400 mg divided in at least 4 doses per day of standard carbidopa/levodopa or divided in at least 3 doses per day of extended-release carbidopa/levodopa capsules at least 4 weeks prior to and concurrently with the treatment with tavapadon provided herein. In one embodiment, the methods provided herein comprise administering a minimum total daily dose of 400 mg divided in at least 4 doses per day of standard levodopa/benserazide or divided in at least 3 doses per day of extended-release levodopa/benserazide capsules at least 4 weeks prior to and concurrently with the treatment with tavapadon provided herein.

Without being bound to any particular theory, in certain embodiments, the methods provided herein improve motor control symptoms while minimizing adverse events (AEs) that may be mechanistically linked to D2/D3 dopamine receptor agonists, including L-Dopa. In one embodiment, the motor control symptoms that may be mechanistically linked to D2/D3 dopamine receptor agonists include dose-limiting hypotension, impulse control disorders, sleep disorders, and, potentially, some forms of hallucinations. In one embodiment, the motor control symptoms that may be mechanistically linked to D2/D3 dopamine receptor agonists include motor fluctuations, which are an alternation between “on” periods when motor symptoms are well controlled and “off” periods when motor symptoms are poorly controlled and the occurrence of dyskinesia or abnormal involuntary movements are heightened.

### 5. BRIEF DESCRIPTION OF THE FIGURES

The foregoing and other objects, features, and advantages will be apparent from the following description of particular embodiments of the invention, as illustrated in the accompanying drawings. The drawings are not necessarily to scale, emphasis instead being placed upon illustrating the principles of various embodiments of the invention.

FIG. 1 provides a schematic for a dose escalation study for tavapadon.

FIG. 2 provides a plot showing the change from baseline in MDS-UPDRS (Movement Disorder Society—Unified Parkinson's Disease Rating Scale) part II+III score for placebo and tavapadon dosing groups.

FIG. 3 provides a plot showing the change from baseline in MDS-UPDRS part II score for placebo and tavapadon dosing groups.

FIG. 4 provides a plot of percentage of PGIC (Patient Global Impression of Change) responders over time.

FIG. 5 provides a plot showing an increase in the daily “on” time without troublesome dyskinesia with tavapadon as an adjunctive therapy to levodopa as compared to placebo.

FIG. 6 provides a plot showing a reduction in the daily “off” time as compared to placebo.

FIG. 7 provides a plot showing a reduction in the total score of MDS-UPDRS Part II with tavapadon as an adjunctive therapy to levodopa as compared to placebo.

FIG. 8 provides a plot showing MDS UPDRS Part I (Non-Motor Aspects of Experiences of Daily Living) Scores for the TemPo-4 trial.

FIG. 9 provides a plot showing MDS UPDRS Parts II (Motor Aspects of Experiences of Daily Living) Scores for the TemPo-4 trial.

FIG. 10 provides a plot showing MDS UPDRS Part III (Motor Examination) Scores for the TemPo-4 trial.

FIG. 11 provides a plot showing the Mean (SEM) On-time without Troublesome Dyskinesia in in TemPo-4 Patients on Daily L-Dopa at the Time of Enrollment.

FIG. 12 provides a plot showing the Mean (SEM) Off-time in TemPo-4 Patients on Daily L-Dopa at the Time of Enrollment.

### 6. DETAILED DESCRIPTION

Provided herein are methods of treating Parkinson's disease (PD) in a subject having PD. Methods disclosed herein comprise administering an escalating dose regimen of tavapadon to the subject.

Exemplary regimens for administering an escalating dose of tavapadon are described elsewhere herein. The methods provided herein for selecting patients, determining the outcome of these methods, and/or serving as criteria in any way for these methods are described in elsewhere herein. Therefore, the methods provided herein include all permutations, including modifications, patients, dosing regimens, diagnostic and PD staging criteria, and therapeutic outcomes as described above and below.

As used in this disclosure including the claims, the singular forms “a,” “an” and “the” include plural referents unless the context clearly dictates otherwise. The terms “a” (or “an”), as well as the terms “one or more,” and “at least one” can be used interchangeably herein unless the context clearly dictates otherwise.

As used herein and unless otherwise indicated, the terms “treat,” “treating,” “treatment,” and “ameliorating” are used interchangeably herein, and mean an alleviation, of PD, or one or more of the symptoms associated with PD, or slowing or halting of further progression or worsening of those symptoms, or alleviating or eradicating the cause(s) of PD.

As used herein, “adverse event” refers to any untoward medical occurrence in a patient or clinical trial subject, temporally associated with the use of trial intervention, whether or not considered related to the trial intervention.

Exemplary adverse events include nausea, headache, fatigue, dry mouth, paraesthesia, hypotension, abnormal dreams, insomnia, hypoaethesia, naropharyngitis, and decreased appetite. In one embodiment, adverse event is nausea, headache, dizziness, fatigue, dysgeusia, dyspepsia, vomiting, dry mouth, or orthostatic hypotension.

Further exemplary adverse events include:
any abnormal laboratory test results (hematology, clinical chemistry, or urinalysis) or other safety assessments (e.g., ECG, radiological scans, vital signs measurements), including those that worsen from baseline, considered clinically significant in the medical and scientific judgment of the investigator;
exacerbation of a chronic or intermittent pre-existing condition including either an increase in frequency and/or intensity of the condition;
new conditions detected or diagnosed after trial intervention administration even though it may have been present before the start of the trial, signs, symptoms, or the clinical manifestations of a suspected drug-drug interaction;
signs, symptoms, or the clinical manifestations of a suspected overdose of either trial intervention or a concomitant medication. “Lack of efficacy” or “failure of expected pharmacological action” per se will not be reported as an AE or SAE/AESI. Such instances will be captured in the efficacy assessments.

The term “about” means within 20%, within 15%, within 10%, within 9%, within 8%, within 7%, within 6%, within 5%, within 4%, within 3%, within 2%, within 1%, or less variation of a given value or range. As used herein, a phrase such as “about A,” includes and is deemed to also recite “A.” As used herein, a phrase such as “about A to about B” includes and is deemed to also recite “A to B.”

### 6.1 Tavapadon

Provided herein are methods for the treatment of a subject suffering from Parkinson's disease, comprising administering tavapadon in an escalating dose schedule to the subject.

Tavapadon, also known as PF-06649751 or CVL-751, has the below chemical formula:

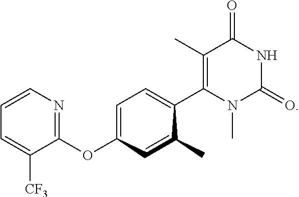

The IUPAC name for tavapadon is S-(−)-1,5-dimethyl-6-[2-methyl-4-(3-trifluoromethyl-pyridin-2-yloxy)-phenyl]-1H-pyrimidine-2,4-dione.

### 6.2 Methods of Treatment

In certain embodiments, provided herein are methods for treating Parkinson's Disease in a patient, the methods comprising orally administering tavapadon to the patient. In one embodiment, the Parkinson's Disease is an early Parkinson's Disease. In one embodiment, the Parkinson's Disease is an advanced Parkinson's Disease. The criteria for early and advanced Parkinson's Disease are known to one of skill in the art and described elsewhere herein. An exemplary criteria are based on the Hoehn and Yahr scale.

In one embodiment, provided herein is a method for treating Parkinson's Disease in a patient, wherein the method comprises: a titration phase comprising orally administering tavapadon in escalating doses to the patient, an adjustment phase following the titration phase, wherein the dose is adjusted based on patient tolerability, and a maintenance phase wherein the patient is administered the highest tolerated dose achieved during the adjustment phase.

In one embodiment, the method provided herein comprises:
a titration phase comprising orally administering to the patient a daily dose of tavapadon in eight sequential dose escalation steps 1-8, wherein
   step 2 comprises administering to the patient a dose of 0.5 mg per day of tavapadon on days 5-8 of treatment,
   step 6 comprises administering to the patient a dose of 2.25 mg per day of tavapadon on days 21-24 of treatment,
   step 8 comprises administering to the patient a dose of a therapeutically effective amount of 5 mg per day of tavapadon,
   thereby treating the Parkinson's Disease.

In one embodiment of the method, the titration phase further comprises maintaining a dose of 3 mg per day of tavapadon once daily for a period of 7 or more days before increasing the dose of tavapadon.

In one embodiment of the method, the titration phase further comprises maintaining the 3 mg of tavapadon once daily dose for at least 15 days before increasing the dose of tavapadon.

In one embodiment of the method, the titration phase further comprises not exceeding the dose of 3 mg of tavapadon once daily until at least 40 days from the commencement of the titration phase.

In one embodiment, the method further comprises an adjustment phase following the titration phase, wherein the adjustment phase comprises:
step 9 comprising administering to the patient a dose of 10 mg per day dose of tavapadon, and
step 10 comprising administering to the patient a therapeutically effective amount of 15 mg per day dose of tavapadon;
wherein the dose of tavapadon is adjusted over the range of a therapeutically effective amount of 5, 10 or 15 mg per day of tavapadon in step 9 and step 10 based on patient tolerability.

In one embodiment, the method further comprises an adjustment phase following the titration phase, wherein the adjustment phase comprises:
step 9 comprising administering to the patient a dose of 10 mg per day dose of tavapadon,
wherein a patient who is unable to increase the dose from 5 mg to 10 mg on the first day of step 9 due to tolerability issues will begin a maintenance phase at the patient's maximum tolerated dose of 5 mg per day.

In one embodiment, the method further comprises a maintenance phase following the adjustment phase, wherein the maintenance phase comprises administering to the patient a therapeutically effective amount of 5, 10 or 15 mg per day dose of tavapadon, wherein the patient is administered the highest tolerated dose achieved during the adjustment phase.

In one embodiment, the dose administered to the patient in the maintenance phase is a maximum tolerated dose of tavapadon.

In one embodiment, the method comprises administering tavapadon to the patient in a dose of 3 or fewer oral pills.

In one embodiment, the dose of tavapadon administered to the patient does not exceed a therapeutically effective amount of 5 mg per day until after at least 56 days of the titration phase.

In one embodiment of the method, the titration phase is fewer than 57 days in duration.

In one embodiment, the method comprises administering to the patient a dose of a therapeutically effective amount of 5 mg per day tavapadon from day 41 of the titration phase.

In one embodiment, the method comprises administering to the patient a 0.25 mg per day dose of tavapadon for days 1-4 of step 1 of the titration phase.

In one embodiment, the method comprises administering to the patient a 0.75 mg per day dose of tavapadon for days 9-12 of step 3 of the titration phase.

In one embodiment, the method comprises administering to the patient a 1.0 mg per day dose of tavapadon for days 13-16 of step 4 of the titration phase.

In one embodiment, the method comprises administering to the patient a 1.5 mg per day dose of tavapadon for days 17-20 of step 5 of the titration phase.

In one embodiment, the method comprises administering to the patient a 3 mg per day dose of tavapadon for days 25-40 of step 7 of the titration phase.

In one embodiment, the method comprises administering to the patient a dose of a therapeutically effective amount of 5 mg per day of tavapadon for days 41-56 of step 8 of the titration phase.

In one embodiment, the method comprises administering to the patient a therapeutically effective amount of 5 mg per day of tavapadon during the maintenance phase.

In one embodiment, the method provided herein comprises administering to the patient a therapeutically effective amount of 15 mg per day of tavapadon during the maintenance phase.

In certain embodiments, the methods provided herein comprise:
a titration phase comprising orally administering to the patient:
0.25 mg per day dose of tavapadon for days 1-4 of the titration phase;
0.5 mg per day dose of tavapadon for days 5-8 of the titration phase;
0.75 mg per day dose of tavapadon for days 9-12 of the titration phase;
1.0 mg per day dose of tavapadon for days 13-16 of the titration phase;
1.5 mg per day dose of tavapadon for days 17-20 of the titration phase;
2.25 mg per day dose of tavapadon for days 21-24 of the titration phase;
3 mg per day dose of tavapadon for days 25-40 of the titration phase; and
a therapeutically effective amount of 5 mg per day dose of tavapadon for days 41-56 of the titration phase;
thereby treating the Parkinson's Disease.

In certain embodiments, the methods provided herein comprise:
i. a titration phase comprising orally administering to the patient:
   0.25 mg per day dose of tavapadon for days 1-4 of the titration phase;
   0.5 mg per day dose of tavapadon for days 5-8 of the titration phase;
   0.75 mg per day dose of tavapadon for days 9-12 of the titration phase;
   1.0 mg per day dose of tavapadon for days 13-16 of the titration phase;
   1.5 mg per day dose of tavapadon for days 17-20 of the titration phase;
   2.25 mg per day dose of tavapadon for days 21-24 of the titration phase;
   3 mg per day dose of tavapadon for days 25-40 of the titration phase; and
   a therapeutically effective amount of 5 mg per day dose of tavapadon for days 41-56 of the titration phase;
ii. followed by an adjustment phase comprising orally administering to the patient:
   a therapeutically effective amount of 10 mg per day dose of tavapadon, and
   a therapeutically effective amount of 15 mg per day dose of tavapadon,
   wherein the dose of tavapadon is adjusted over the range of 5, 10 or 15 mg based on patient tolerability; and
iii. followed by a maintenance phase comprising orally administering to the patient:
   a therapeutically effective amount of 5, 10 or 15 mg per day dose of tavapadon;
wherein the patient is administered the highest tolerated dose achieved during the adjustment phase and the maintenance phase maintains the patient on a steady dose of tavapadon,
   thereby treating the Parkinson's Disease.

In certain embodiments, the methods provided herein comprise:
i. a titration phase comprising orally administering to the patient:
   0.25 mg per day dose of tavapadon for days 1-4 of the titration phase;
   0.5 mg per day dose of tavapadon for days 5-8 of the titration phase;
   0.75 mg per day dose of tavapadon for days 9-12 of the titration phase;
   1.0 mg per day dose of tavapadon for days 13-16 of the titration phase;
   1.5 mg per day dose of tavapadon for days 17-20 of the titration phase;
   2.25 mg per day dose of tavapadon for days 21-24 of the titration phase;
   3 mg per day dose of tavapadon for days 25-40 of the titration phase; and
   a therapeutically effective amount of 5 mg per day dose of tavapadon for days 41-56 of the titration phase;
ii. followed by an adjustment phase comprising orally administering to the patient: a therapeutically effective amount of 10 mg per day dose of tavapadon, and
   wherein a patient who is unable to increase the dose from 5 mg to 10 mg on the first day of step 9 due to tolerability issues will begin a maintenance phase at the patient's maximum tolerated dose of 5 mg per day; and
iii. followed by a maintenance phase comprising orally administering to the patient:
   a therapeutically effective amount of 5, 10 or 15 mg per day dose of tavapadon;
wherein the patient is administered the highest tolerated dose achieved during the adjustment phase and the maintenance phase maintains the patient on a steady dose of tavapadon,
   thereby treating the Parkinson's Disease.

In one embodiment, the methods provided herein comprise administering to the patient a therapeutically effective amount of 5 mg per day of tavapadon.

In one embodiment, the methods provided herein comprise administering to the patient a therapeutically effective amount of 10 mg per day of tavapadon.

In one embodiment, the methods provided herein comprise administering to the patient a therapeutically effective amount of 15 mg per day of tavapadon.

In one embodiment, the methods provided herein comprise administering to the patient a safe and effective treatment of Parkinson's Disease, wherein the method comprises:
a titration phase comprising orally administering escalating tavapadon doses to the patient, wherein the dose of tavapadon does not exceed a therapeutically effective amount of 5 mg per day until after at least 40 days from the commencement of the titration phase, and
a maintenance phase after the titration phase, comprising orally administering once daily to the patient a therapeutically effective amount of a maintenance dose of tavapadon.

In one embodiment, the maintenance dose is selected from the group consisting of a therapeutically effective amount of 5, 10, and 15 mg tavapadon.

In one embodiment, the titration phase further comprises not exceeding a therapeutically effective amount of 10 mg of tavapadon once daily until after at least 56 days from the commencement of the titration phase.

In one embodiment, the titration phase further comprises not exceeding a therapeutically effective amount of 15 mg of tavapadon once daily until after at least 72 days from the commencement of the titration phase.

In one embodiment, provided herein is a method for treating Parkinson's Disease in a patient, wherein the method comprises a titration phase comprising orally administering to the patient a daily dose of tavapadon in dose escalation steps, wherein the dose of tavapadon does not exceed a therapeutically effective amount of 5 mg per day until after at least day 56 of the titration phase, thereby treating the Parkinson's Disease.

In one embodiment provided herein is a method for treating Parkinson's Disease in a patient, the method comprising orally administering tavapadon in escalating doses to the patient, wherein the escalating doses comprise a therapeutically effective amount of a 0.25 mg dose of tavapadon per day for days 1-4 of a treatment followed by a therapeutically effective amount of a 10 mg dose of tavapadon per day from day 57 of the treatment.

In one embodiment provided herein is a method for treating Parkinson's Disease in a patient, the method comprising orally administering tavapadon in escalating doses to the patient, wherein the escalating doses comprise a therapeutically effective amount of a 0.25 mg dose of tavapadon per day for days 1-4 of a treatment followed by a therapeutically effective amount of a 15 mg dose of tavapadon per day from day 73 of the treatment.

In one embodiment of the method, the escalating doses further comprise a therapeutically effective amount of a 15 mg dose of tavapadon per day for days 73-189 of the treatment.

In one embodiment, the method comprises maintaining the patient on a therapeutically effective amount of a 5, mg, 10 mg or 15 mg dose of tavapadon per day.

In certain embodiments, the methods provided herein comprise orally administering tavapadon in escalating doses to the patient, wherein the escalating doses comprise a 0.25 mg dose of tavapadon followed by a therapeutically effective amount of a 5 mg dose of tavapadon per day from day 41 of a treatment.

In certain embodiments, the methods provided herein comprise orally administering tavapadon in escalating doses to the patient, wherein the escalating doses comprise a 0.25 mg dose of tavapadon followed by a therapeutically effective amount of a 10 mg dose of tavapadon per day from day 57 of the treatment.

In certain embodiments, the methods provided herein comprise orally administering tavapadon in escalating doses to the patient, wherein the escalating doses comprise a 0.25 mg dose of tavapadon followed by a therapeutically effective amount of a 15 mg dose of tavapadon per day from day 73 of a treatment.

In certain embodiments of the methods provided herein, the patients who cannot tolerate the tavapadon dose of 5 mg per day are discontinued from the treatment.

In certain embodiments of the methods provided herein, the patients who cannot tolerate the tavapadon dose 10 mg per day are administered the lower tavapadon dose of 5 mg per day. In certain embodiments of the methods provided herein, the patients who cannot tolerate the tavapadon dose of 10 mg per day are discontinued from the treatment.

In certain embodiments of the methods provided herein, the patients who cannot tolerate the tavapadon dose 15 mg per day are administered the lower tavapadon dose of 10 mg per day. In certain embodiments of the methods provided herein, the patients who cannot tolerate the tavapadon dose 10 mg per day are administered the lower tavapadon dose of 5 mg per day. In certain embodiments of the methods provided herein, the patients who cannot tolerate the tavapadon dose of 15 mg per day are discontinued from the treatment.

In certain embodiments, the dose escalation or step-wise increase is an increase in the amount per day of tavapadon administered to the patient. In certain embodiments, the period of time over which completion of the step-wise increase occurs (the dose escalation period of time) is 41 days, 57 days, or 73 days. In certain embodiments, a step-wise increase in the amount of tavapadon administered occurs (or is scheduled to occur) on days 5, 9, 17, 21, 25, 41, 57 and 73, during the dose escalation period of time. In certain embodiments, the final amount of tavapadon administered to the patient at the end of the dose escalation period of time comprises an effective amount of tavapadon.

In one embodiment of the method provided herein, tavapadon dose of 5 mg, once daily, provides a statistically significant and clinical meaningful improvement over placebo on the primary endpoint of change from baseline to Week 26 in MDS-UPDRS Parts II+III combined score.

In one embodiment of the method provided herein, tavapadon dose of 15 mg, once daily, provides a statistically significant and clinical meaningful improvement over placebo on the primary endpoint of change from baseline to Week 26 in MDS-UPDRS Parts II+III combined score.

In one embodiment, the combined score in the patient is improved by −12.1, wherein the patient is administered a therapeutically effective amount of 15 mg per day dose of tavapadon in the maintenance phase.

In one embodiment, the combined score in the patient is improved by −11.5, wherein the patient is administered a therapeutically effective amount of 5 mg per day dose of tavapadon in the maintenance phase.

In one embodiment of the method provided herein, tavapadon dose of 5 mg, once daily, provides a statistically significant and clinical meaningful improvement over placebo on the primary endpoint of change from baseline to Week 26 in MDS-UPDRS Part II score.

In one embodiment of the method provided herein, tavapadon dose of 15 mg, once daily, provides a statistically significant and clinical meaningful improvement over placebo on the primary endpoint of change from baseline to Week 26 in MDS-UPDRS Part II score.

In certain embodiments, statistically significant and clinical meaningful improvement in MDS-UPDRS Parts II+III combined score refers to >3-point decrease from baseline. In certain embodiments, statistically significant and clinical meaningful improvement in MDS-UPDRS Parts II+III combined score refers to >5-point decrease from baseline. In certain embodiments, statistically significant and clinical meaningful improvement in MDS-UPDRS Parts II+III combined score refers to a mean improvement (i.e. reduction) in the combined UPDRS (Parts II+III) from baseline to end of treatment of about −6 to about −13. In certain embodiments, statistically significant and clinical meaningful improvement in MDS-UPDRS Parts II+III combined score refers to a mean improvement (i.e. reduction) in the combined UPDRS (Parts II+III) from baseline to end of treatment of about −7 to about −12. In certain embodiments, statistically significant and clinical meaningful improvement in MDS-UPDRS Parts II+III combined score refers to a mean improvement (i.e. reduction) in the combined UPDRS (Parts II+III) from baseline to end of treatment of about −3 to about −20. In certain embodiments, statistically significant and clinical meaningful improvement in MDS-UPDRS Parts II+III combined score refers to a mean improvement (i.e. reduction) in the combined UPDRS (Parts II+III) from baseline to end of treatment of about −3, about −4, about −5, about −6, about −7, about −8, about −9, about −10, about −11, about −12, about −13, about −14 or about −15.

In certain embodiments, statistically significant and clinical meaningful improvement in MDS-UPDRS Part II score refers to >0.5-point decrease from baseline. In certain embodiments, statistically significant and clinical meaningful improvement in MDS-UPDRS Part II score refers to >1-point decrease from baseline. In certain embodiments, statistically significant and clinical meaningful improvement in MDS-UPDRS Part II score refers to a mean improvement (i.e. reduction) in the UPDRS (Part II) score from baseline to end of treatment of about −0.5 to about −10. In certain embodiments, statistically significant and clinical meaningful improvement in MDS-UPDRS Part II score refers to a mean improvement (i.e. reduction) in the UPDRS (Part II) score from baseline to end of treatment of about −0.5 to about −3. In certain embodiments, statistically significant and clinical meaningful improvement in MDS-UPDRS Part II score refers to a mean improvement (i.e. reduction) in the UPDRS (Part II) score from baseline to end of treatment of about −0.5, about −1, about −1.5, about −2, about −2.5, about −3, about −3.5, about −4, about −4.5, about −5, about −5.5, about −6, about −7, about −8, about −9 or about −10. In certain embodiments, statistically significant and clinical meaningful improvement in MDS-UPDRS Part II score refers to a mean improvement (i.e. reduction) in the UPDRS (Part II) score from baseline to end of treatment of about −1, about −1.6 or about −2.2. In certain embodiments, statistically significant and clinical meaningful improvement in MDS-UPDRS Part II score refers to a mean improvement (i.e. reduction) in the UPDRS (Part II) score from baseline to end of treatment of about −1.1, about −1.7 or about −2.4.

In one embodiment, the MDS-UPDRS Part II score in the patient is improved by −2.6, wherein the patient is administered a therapeutically effective amount of 15 mg per day dose of tavapadon in the maintenance phase.

In one embodiment, the MDS-UPDRS Part II score in the patient is improved by −2.5, wherein the patient is administered a therapeutically effective amount of 5 mg per day dose of tavapadon in the maintenance phase.

In one embodiment, the method herein provides an increased daily “on” time without troublesome dyskinesia as adjunctive therapy to levodopa in patients with advanced Parkinson's Disease.

In one embodiment, the method herein provides an increased daily “on” time without troublesome dyskinesia as adjunctive therapy to levodopa by about 1.1 hours as compared to placebo with p<0.0001 (Cohen's D effect size 0.4).

In one embodiment, the method herein provides an increased daily “on” time without troublesome dyskinesia as adjunctive therapy to levodopa at Week 26 by about 1.1 hours as compared to placebo with p<0.0001 (Cohen's D effect size 0.4).

In one embodiment, the method herein provides a reduced daily “off” time as adjunctive therapy to levodopa in patients with advanced Parkinson's Disease. In one embodiment, the method herein provides a reduced daily “off” time as adjunctive therapy to levodopa by 0.95 hours as compared to placebo with p=0.0006 (Cohen's D effect size 0.35). In one embodiment, the method herein provides a reduced daily “off” time as adjunctive therapy to levodopa at Week 26 by 0.95 hours as compared to placebo with p=0.0006 (Cohen's D effect size 0.35).

In certain embodiments, the methods provided herein improve motor control symptoms while minimizing adverse events (AEs) that may be mechanistically linked to D2/D3 dopamine receptor agonists, including L-Dopa. In certain embodiments, the methods provided herein improve the motor control symptoms that may be mechanistically linked to D2/D3 dopamine receptor agonists include dose-limiting hypotension, impulse control disorders, sleep disorders, and, potentially, some forms of hallucinations. In certain embodiments, the methods provided herein improve the motor control symptoms that may be mechanistically linked to D2/D3 dopamine receptor agonists include motor fluctuations, which are an alternation between “on” periods when motor symptoms are well controlled and “off” periods when motor symptoms are poorly controlled and the occurrence of dyskinesia or abnormal involuntary movements.

In certain embodiments, the treatment with tavapadon provided herein results in a reduced incidence of at least one adverse event compared to a treatment method wherein a therapeutically effective amount of at least 5 mg of tavapadon is administered once daily prior to 40 days from commencement of treatment.
the treatment with tavapadon provided herein results in a reduced incidence of at least one adverse event selected from the group consisting of nausea, headache, fatigue, dry mouth, paraesthesia, hypotension, abnormal dreams, insomnia, hypoaethesia, naropharyngitis, and decreased appetite.

In certain embodiments, the treatment with tavapadon provided herein results in a reduced incidence of at least one adverse event selected from the group consisting of nausea, headache, dizziness, fatigue, dysgeusia, dyspepsia, vomiting, dry mouth, and orthostatic hypotension. In certain embodiments, the treatment with tavapadon provided herein results in a reduced incidence of nausea.

### 6.3 Combination Therapies

In combination with the PD therapies with tavapadon disclosed herein, a subject diagnosed with or PD can also be treated with any agent that is known in the art for treatment of PD. In certain embodiments, provided herein are methods of administering one or more agents that treat or prevent PD (e.g., based on the agent's product label or known to one skilled in the art e.g., Murakami et al., Intern Med. 2023 Jan. 1; 62(1):33-42). In certain embodiments, the one or more agents are selected from dopaminergic agents, COMT inhibitors, MAO-B inhibitors, amantadine, istradefylline, and anticholinergic drugs. In some embodiments, an agent that treats or prevents PD suitable for use herein can comprise a dopaminergic agent. In some embodiments, an agent that treats or prevents PD suitable for use herein can comprise a COMT (catechol-O-methyltransferase) inhibitor. In some embodiments, an agent that treats or prevents PD suitable for use herein can comprise a MAO-B (monoamine oxidase—B) inhibitor. In some embodiments, an agent that treats or prevents PD suitable for use herein can comprise an anticholinergic. In some embodiments, an agent that treats or prevents PD suitable for use herein can comprise an amantadine. Additional non-limiting examples of agents that treat and/or prevent PD include levodopa, levodopa-carbidopa, pramipexole, ropinirole, rotigotine, apomorphine, selegiline, rasagiline, safinamide, entacapone, opicapone, tolcapone, amantadine, apomorphine, istradefylline, benzotropine, triheyphenidyl, pimavanserin, or a combination thereof. In certain embodiments, methods disclosed herein can include administering a regimen of tavapadon in combination with one or more agents that treats or prevents PD. In certain embodiments, methods disclosed herein can include administering a regimen of tavapadon in combination with one or more of levodopa, levodopa-carbidopa, pramipexole, ropinirole, rotigotine, apomorphine, selegiline, rasagiline, safinamide, entacapone, opicapone, tolcapone, amantadine, apomorphine, istradefylline, benzotropine, triheyphenidyl, pimavanserin, derivatives thereof, or any combination thereof.

In certain embodiments, the methods provided herein comprise administering one or more agents that treat PD concurrently with or prior to the treatment with tavapadon provided herein. In certain embodiments, the methods provided herein comprise administering one or more agents that treat PD concurrently with the treatment with tavapadon provided herein. In certain embodiments, the methods provided herein comprise administering one or more agents that treat PD prior to the treatment with tavapadon provided herein.

In certain embodiments of the methods provided herein, the patient is previously and/or concurrently treated with a therapeutically effective dose of one or more monoamine oxidase B (MAO-B) inhibitors. In certain embodiments of the methods provided herein, the patient is previously and/or concurrently treated with a therapeutically effective dose of one or more monoamine oxidase B (MAO-B) inhibitors, provided that the treatment with the one or more monoamine oxidase B (MAO-B) inhibitors was initiated >90 days prior to the treatment with tavapadon.

In certain embodiments of the methods provided herein, the patient is previously treated with a therapeutically effective dose of one or more monoamine oxidase B (MAO-B) inhibitors. In certain embodiments of the methods provided herein, the patient is previously treated with a therapeutically effective dose of one or more monoamine oxidase B (MAO-B) inhibitors, provided that the treatment with the one or more monoamine oxidase B (MAO-B) inhibitors was initiated >90 days prior to the treatment with tavapadon and the dose of the one or more monoamine oxidase B (MAO-B) inhibitors is stable for the duration of the treatment.

In certain embodiments of the methods provided herein, the patient is concurrently treated with a therapeutically effective dose of one or more monoamine oxidase B (MAO-B) inhibitors. In certain embodiments of the methods provided herein, the patient is concurrently treated with a therapeutically effective dose of one or more monoamine oxidase B (MAO-B) inhibitors, provided that the treatment with the one or more monoamine oxidase B (MAO-B) inhibitors was initiated >90 days prior to the treatment with tavapadon and the dose of the one or more monoamine oxidase B (MAO-B) inhibitors is stable for the duration of the treatment.

In certain embodiments, the methods provided herein comprise administering L-Dopa concurrently with or prior to the treatment with tavapadon. In certain embodiments, the methods provided herein comprise administering L-Dopa concurrently with or prior to the treatment with tavapadon in patient with advanced PD. In certain embodiments, the methods provided herein comprise administering L-Dopa concurrently with and prior to the treatment with tavapadon. In certain embodiments, the methods provided herein comprise administering standard carbidopa/levodopa or extended-release carbidopa/levodopa capsules concurrently with or prior to the treatment with tavapadon. In certain embodiments, the methods provided herein comprise administering standard carbidopa/levodopa or extended-release carbidopa/levodopa capsules concurrently with and prior to the treatment with tavapadon. In one embodiment, the methods provided herein comprise administering a stable dose of L-Dopa at least 4 weeks prior to and concurrently with the treatment with tavapadon. In one embodiment, the methods provided herein comprise administering a minimum total daily dose of 400 mg of L-Dopa at least 4 weeks prior to and concurrently with the treatment with tavapadon. In one embodiment, the methods provided herein comprise administering a minimum total daily dose of 400 mg divided in at least 4 doses per day of standard carbidopa/levodopa or divided in at least 3 doses per day of extended-release carbidopa/levodopa capsules at least 4 weeks prior to and concurrently with the treatment with tavapadon. In one embodiment, the methods provided herein comprise administering a minimum total daily dose of 400 mg divided in at least 4 doses per day of standard levodopa/benserazide or divided in at least 3 doses per day of extended-release levodopa/benserazide capsules at least 4 weeks prior to and concurrently with the treatment with tavapadon.

In certain embodiments of the methods provided herein, the patient is not previously and/or concurrently treated with other Parkinson's Disease medications. In certain embodiments of the methods provided herein, the patient is not previously or concurrently treated with other Parkinson's Disease medications. In certain embodiments of the methods provided herein, the patient is not previously treated with other Parkinson's Disease medications. In certain embodiments of the methods provided herein, the patient is not concurrently treated with other Parkinson's Disease medications.

### 6.4 Patient Population

Parkinson's disease (PD) is a chronic and progressive neurodegenerative condition characterized by the loss dopaminergic neurons, and associated levels of the neurotransmitter dopamine, in the brain. In general, PD diagnosed as beginning after age 50 is considered late-onset disease whereas a PD diagnosis before age 20 is considered early-onset disease and is sometimes referred to as juvenile-onset Parkinson disease. Most PD cases are classified as sporadic with unknown etiology; however, about 15% of PD cases are considered to be familial. Familial PD cases can result from one or more mutations in the following genes: GBA, LRRK2, PARK7, PINK1, PRKKN, SNCA, and VPS3. Karen Nuyteman et al, Hum Mutat. 2010 July; 31(7): 763-780.

Described herein are methods for treating PD in a subject diagnosed with PD. The term “subject” and “patient” are used interchangeably throughout the present disclosure. Subjects for whom the methods provided herein can be used can be diagnosed and selected for based on assessing diagnostic criteria and/or assessing markers associated with PD according to the methods described elsewhere herein. In certain embodiments, subjects for whom the methods provided herein can be used may have a particular stage of PD at the time of treatment.

In certain embodiments, a subject to the methods disclosed herein is a mammal. In some embodiments, the subject is a human subject. In some embodiments, the subject is an adult human subject. In some embodiments, the subject is a female human subject. In some embodiments, the subject is a male human subject. In some embodiments, the human subject is between about 18 to about 90 years of age. In some embodiments, the human subject is over 18, over 20, over 25, over 30, over 35, over 50, over 55, over 60, over 65, over 70, over 75, over 80, over 85, or over 90 years of age. In certain embodiments, the subject is about 40 to 80 years of age.

In certain embodiments, the methods provided herein are for treatment of a subject diagnosed as having PD according to UK Parkinson's Disease Society Brain Bank (UKBB) criteria (described elsewhere herein).

In certain embodiments, the methods provided herein are for treatment of a subject diagnosed as having possible PD or probable PD according to Gelb criteria (described elsewhere herein). In certain embodiments, the methods provided herein are for treatment of a subject diagnosed as having possible PD. In certain embodiments, the methods provided herein are for treatment of a subject diagnosed as having probable PD.

In certain embodiments, the methods provided herein are for treatment of a subject diagnosed as having clinically established PD or clinically probable PD according to the Movement Disorder Society (MDS) PD criteria (described elsewhere herein). In certain embodiments, the methods provided herein are for treatment of a subject diagnosed as having clinically established PD. In certain embodiments, the methods provided herein are for treatment of a subject diagnosed as having clinically probable PD.

In some embodiments, the subject is a human subject recently diagnosed with PD (e.g., diagnosed as having first symptoms within 3 months to 3 years from start of administration of a PD therapy contemplated in the present disclosure). In some embodiments, the subject is a human subject having a diagnosis of PD at any time prior to the administration of a PD therapy contemplated in the present disclosure.

In certain embodiments, patients for whom the methods provided herein can be used include PD patients at various disease stages. In certain embodiments, the subject has a diagnosis of PD that is consistent with the UK Parkinson's Disease Society Brain Bank diagnostic criteria. In certain embodiments, the subject having more than one affected relative (Step 2 UK Parkinson's Disease Society Brain Bank exclusion criterion) but meeting all other criteria consistent with the UK criteria will be considered to have met eligibility criteria for a diagnosis of PD.

In some embodiments, PD patients for whom the methods provided herein can be used can be staged according to the Hoehn and Yahr scale of staging. In some embodiments, PD patients for whom the methods provided herein can be used are stage 1 PD patients, stage 1.5 PD patients, stage 2 PD patients, stage 2.5 PD patients, stage 3 PD patients, stage 4 PD patients, and/or stage 5 PD patients according to the Hoehn and Yahr scale/modified Hoehn and Yahr scale of staging. In some embodiments, PD patients for whom the methods provided herein can be used are stage 1 PD patients, stage 1.5 PD patients, or stage 2 PD patients according to the modified Hoehn and Yahr scale of staging. In some embodiments, PD patients for whom the methods provided herein can be used are stage 1 PD patients according to the Hoehn and Yahr scale/modified Hoehn and Yahr scale of staging. In some embodiments, PD patients for whom the methods provided herein can be used are stage 1.5 PD patients according to the modified Hoehn and Yahr scale of staging. In some embodiments, PD patients for whom the methods provided herein can be used are stage 2 PD patients according to the Hoehn and Yahr scale/modified Hoehn and Yahr scale of staging. In some embodiments, PD patients for whom the methods provided herein can be used are stage 2.5 PD patients according to the Hoehn and Yahr scale/modified Hoehn and Yahr scale of staging. In some embodiments, PD patients for whom the methods provided herein can be used are stage 3 PD patients according to the Hoehn and Yahr scale/modified Hoehn and Yahr scale of staging. In some embodiments, PD patients for whom the methods provided herein can be used are stage 2, 2.5, or 3 PD patients in the “on” state according to modified Hoehn and Yahr stage.

In certain embodiments, patients for whom the methods provided herein can be used include PD patients in the early stages of PD according to the Hoehn and Yahr scale of staging. In certain embodiments, patients for whom the methods provided herein can be used include PD patients in the middle stages of PD according to the Hoehn and Yahr scale of staging. In certain embodiments, patients for whom the methods provided herein can be used include PD patients in advanced stages of PD according to the Hoehn and Yahr scale of staging.

In certain embodiments, the subject has PD diagnosed according to Movement Disorder Society (MDS) clinical diagnostic criteria as described in Postuma et al. Mov Disord. 2015; 30:1591-601. In one embodiment, the subject has an MDS-UPDRS Part II score≥2 and Part III score≥10.

In some embodiments, the human subject is a naïve subject (i.e., a subject that has not been treated for PD). In some embodiments, the human subject has been previously treated with an agent that is used for treating PD. In some embodiments, the human subject was not responsive to previous PD treatment(s). In some embodiments, the subject is unresponsive to at least one treatment for PD. In some embodiments, the subject is unresponsive to several treatments of PD. In some embodiments, the subject has sporadic PD. In some embodiments, the subject has familial PD. In some embodiments, the subject has a genetic mutation associated with PD (e.g., a mutation in GBA, LRRK2, PARK7, PINK1, PRKN, SNCA, and/or VPS3).

In some embodiments, the subject does not have a history or clinical features consistent with essential tremor, atypical or secondary parkinsonian syndrome (including, but not limited to, progressive supranuclear palsy, multiple system atrophy, cortico-basal degeneration, or drug-induced or poststroke parkinsonism). In certain embodiments, the subject is treated with other Parkinson's Disease medications concurrently with or prior to the treatment with tavapadon provided herein. Exemplary medications for Parkinson's Disease are described elsewhere herein.

In certain embodiments, the subject is treated with a therapeutically effective dose of one or more monoamine oxidase B (MAO-B) inhibitors concurrently with or prior to the treatment with tavapadon provided herein. In certain embodiments, the subject is treated with a therapeutically effective dose of one or more monoamine oxidase B (MAO-B) inhibitors concurrently with or prior to the treatment with tavapadon provided herein, provided that the treatment with the one or more monoamine oxidase B (MAO-B) inhibitors was initiated >90 days prior to the treatment with tavapadon.

In certain embodiments, the subject is treated with a therapeutically effective dose of one or more monoamine oxidase B (MAO-B) inhibitors prior to the treatment with tavapadon provided herein. In certain embodiments, the subject is treated with a therapeutically effective dose of one or more monoamine oxidase B (MAO-B) inhibitors prior to the treatment with tavapadon provided herein, provided that the treatment with the one or more monoamine oxidase B (MAO-B) inhibitors was initiated >90 days prior to the treatment with tavapadon and the dose of the one or more monoamine oxidase B (MAO-B) inhibitors is stable for the duration of the treatment.

In certain embodiments, the subject is treated with a therapeutically effective dose of one or more monoamine oxidase B (MAO-B) inhibitors concurrently with the treatment with tavapadon provided herein. In certain embodiments, the subject is treated with a therapeutically effective dose of one or more monoamine oxidase B (MAO-B) inhibitors concurrently with the treatment with tavapadon provided herein, provided that the treatment with the one or more monoamine oxidase B (MAO-B) inhibitors was initiated >90 days prior to the treatment with tavapadon and the dose of the one or more monoamine oxidase B (MAO-B) inhibitors is stable for the duration of the treatment.

In certain embodiments, the subject is treated with a therapeutically effective dose of L-Dopa concurrently with or prior to the treatment with tavapadon provided herein. In certain embodiments, the subject is treated with a therapeutically effective dose of L-Dopa concurrently with and prior to the treatment with tavapadon provided herein. In one embodiment, the subject is on a stable dose of L-Dopa at least 4 weeks prior to and concurrently with the treatment with tavapadon provided herein. In one embodiment, the subject is on a minimum total daily dose of 400 mg of L-Dopa at least 4 weeks prior to and concurrently with the treatment with tavapadon provided herein. In one embodiment, the subject is on a minimum total daily dose of 400 mg divided in at least 4 doses per day of standard carbidopa/levodopa or divided in at least 3 doses per day of extended-release carbidopa/levodopa capsules at least 4 weeks prior to and concurrently with the treatment with tavapadon provided herein. In one embodiment, the subject is on a minimum total daily dose of 400 mg divided in at least 4 doses per day of standard levodopa/benserazide or divided in at least 3 doses per day of extended-release levodopa/benserazide capsules at least 4 weeks prior to and concurrently with the treatment with tavapadon provided herein.

In some embodiments, the subject has a good response to L-Dopa in the judgment of a medical practitioner.

In certain embodiments, the subject is not treated with levodopa/carbidopa intestinal gel prior to and concurrently with the treatment with tavapadon provided herein. In certain embodiments, the subject is not treated with levodopa/carbidopa intestinal gel prior to or concurrently with the treatment with tavapadon provided herein.

In some embodiments, the subject does not have a history of nonresponse or insufficient response to L-Dopa or 2 or more other antiparkinsonian drugs at therapeutic dosages.

In some embodiments, the subject does not have a previous surgical intervention (e.g., deep brain stimulation) for PD or for whom such a procedure is planned or anticipated during the trial period.

In some embodiments, the subject does not have another acute or chronic disorder of the immune system (e.g., only PD). In some embodiments, the subject does not have another immune related disorder (e.g., only PD). In some embodiments, the subject does not have another chronic or acute inflammatory disorder of the immune system (e.g., only PD). In some embodiments, the subject does not have another disease or condition besides PD. In some embodiments, the subject has another acute or chronic disorder of the immune system (e.g., in addition to PD). In some embodiments, the subject has another immune related disorder (e.g., in addition to PD). In some embodiments, the subject has another chronic or acute inflammatory disorder of the immune system (e.g., in addition to PD). In some embodiments, the subject has another disease or condition in addition to PD.

### 6.5 Pharmaceutical Compositions

Provided herein are pharmaceutical compositions comprising a therapeutically effective amount of tavapadon and a pharmaceutically acceptable carrier.

In one embodiment, the pharmaceutical composition is a tablet suitable for oral administration.

The pharmaceutical composition may be in unit dosage forms suitable for single administration of precise dosages. In some embodiments, the pharmaceutical compositions are in unit dosage forms containing 0.25 mg, 1 mg, and 5 mg of tavapadon.

### 6.6 Methods of Monitoring Efficacy of a PD Therapy

In certain embodiments, provided herein is a method for treating a PD patient comprising administering a first PD therapy to the PD patient; assessing the responsiveness of the PD patient to the first PD therapy; and (a) continue administering the first PD therapy to the PD patient if the PD patient is assessed to be responsive to the first PD therapy, or (b) discontinue administering the first PD therapy to the PD patient if the PD patient is assessed to be non-responsive to the first PD therapy according to the methods disclosed herein.

In certain embodiments, provided herein is a method for treating PD in a patient diagnosed therewith, the method comprising: administering a first PD therapy to the PD patient; assessing the responsiveness of the PD patient to the first PD therapy; and continuing to administer the first PD therapy to the PD patient if the PD patient is assessed to be responsive to the first PD therapy according to the methods disclosed herein.

In certain embodiments of the above method for treating PD, in instances where a patient is found to not be responsive to the first PD therapy, the method further comprises administering to the patient a second PD therapy. In some embodiments, the method may further comprise, following the administering the second PD therapy to the PD patient, assessing the responsiveness of the PD patient to the second PD therapy, and continuing to administer the second PD therapy to the PD patient if the PD patient is assessed to be responsive to the second PD therapy according to the methods disclosed herein.

In certain embodiments provided herein are methods for treating a PD patient comprising administering a combination of PD therapies to the PD patient; assessing the responsiveness of the PD patient to the combination of PD therapies administered; and continue administering the combination of PD therapies to the PD patient if the PD patient is assessed to be responsive to the combination of PD therapies according to the methods disclosed herein.

In certain embodiments, the progression of PD can be measured using the Unified Parkinson's Disease Rating Scale (UPDRS). The UPDRS comprises four parts: 1) mentation, behavior, mood; 2) activities of daily living (determine for “on” or “off”, indicating either a “good” or “bad” day, respectively); 3) motor examination; and 4) complications of therapy (in the past week). One of skill in the art administering the UPDRS scores Parts I to III on a 0-4 rating scale and scores Part IV with “yes” and “no” ratings according to the methods described in Fahn et al., RECENT DEVELOPMENTS IN PARKINSON'S DISEASE, Vol 2. Florham Park, NJ. Macmillan Health Care Information 1987, pp 15 3-163, 293-304. Higher UPDRS scores indicate increased severity with 0 indicating no disability and 260 indicating total disability.

In certain embodiments, a patient administered tavapadon according to the methods disclosed herein has no change in UPDRS score compared to baseline. In certain embodiments, a patient administered tavapadon according to the methods disclosed herein has a decreased UPDRS score compared to baseline. In certain embodiments, a patient administered tavapadon according to the methods disclosed herein has minimal increase (no more than a 0.1 point increase) in UPDRS score compared to baseline.

In certain embodiments, a patient administered tavapadon according to the methods disclosed herein has a UPDRS score decreased by at least about 0.2 points compared to baseline. In certain embodiments, a patient administered tavapadon according to the methods disclosed herein has a UPDRS score decreased by at least about 0.2 points, at least about 0.3 points, at least about 0.4 points, at least about 0.5 points, at least about 0.6 points, at least about 0.7 points, at least about 0.8 points, at least about 0.9 points, at least about 1.0 points, at least about 2.0 points, at least about 2.5 points, at least about 3.0 points, at least about 3.5 points, at least about 4.0 points, at least about 4.5 points, at least about 5.0 points, at least about 5.5 points, at least about 6.0 points, at least about 6.5 points, at least about 7.0 points, at least about 7.5 points, at least about 8.0 points, at least about 8.5 points, at least about 9.0 points, at least about 9.5 points, at least about 10 points, at least about 11 points, at least about 12 points, at least about 13 points, at least about 14 points, at least about 15 points, at least about 16 points, at least about 17 points, at least about 18 points, at least about 19 points, at least about 20 points, at least about 25 points, at least about 30 points, at least about 35 points, at least about 40 points, at least about 45 points, or at least about 50 points compared to baseline. In certain embodiments, a patient administered tavapadon according to the methods disclosed herein has a UPDRS score decreased by about 0.1 to about 10 points, by about 0.1 to about 0.2 points, by about 0.2 to about 0.3 points, by about 0.3 to about 0.4 points, by about 0.4 to about 0.5 points, by about 0.5 to about 0.6 points, by about 0.6 to about 0.7 points, by about 0.7 to about 0.8 points, by about 0.8 to about 0.9 points, by about 0.9 to about 1.0 points, by about 0.1 to about 0.5 points, by about 0.5 to about 1.0 points, by about 0.1 to about 1.0 points, by about 1.0 to about 1.5 points, by about 1.5 to about 2.0 points, by about 2.0 to about 2.5 points, by about 2.5 to about 3.0 points, by about 3.0 to about 3.5 points, by about 3.5 to about 4.0 points, by about 4.0 to about 4.5 points, by about 4.5 to about 5.0 points, by about 5.0 to about 5.5 points, by about 5.5 to about 6.0 points, by about 6.0 to about 6.5 points, by about 6.5 to about 7.0 points, by about 7.0 to about 7.5 points, by about 7.5 to about 8.0 points, by about 8.0 to about 8.5 points, by about 8.5 to about 9.0 points, by about 9.0 to about 9.5 points, by about 9.5 to about 10 points, by about 1.0 to about 5.0 points, by about 5.0 to about 10 points, by about 10 to about 11 points, by about 11 to about 12 points, by about 12 to about 13 points, by about 13 to about 14 points, by about 14 to about 15 points, by about 15 to about 16 points, by about 16 to about 17 points, by about 17 to about 18 points, by about 18 to about 19 points, by about 19 to about 20 points, by about 20 to about 25 points, by about 25 to about 30 points, by about 30 to about 35 points, by about 35 to about 40 points, by about 40 to about 45 points, by about 45 to about 50 points, by about 10 to about 15 points, or by about 15 to about 20 points. In certain embodiments, a patient administered tavapadon according to the methods disclosed herein has a UPDRS score decreased by about 0.1 points, about 0.2 points, about 0.3 points, about 0.4 points, about 0.5 points, about 0.6 points, about 0.7 points, about 0.8 points, about 0.9 points, about 1.0 points, about 2 points, about 2.5 points, about 3 points, about 3.5 points, about 4 points, about 4.5 points, about 5 points, about 5.5 points, about 6 points, about 6.5 points, about 7 points, about 7.5 points, about 8 points, about 8.5 points, about 9 points, about 9.5 points, about 10 points, about 11 points, about 12 points, about 13 points, about 14 points, about 15 points, about 16 points, about 17 points, about 18 points, about 19 points, about 20 points, about 25 points, about 30 points, about 35 points, about 40 points, about 45 points, about 50 points, or more than 50 points compared to baseline.

In certain embodiments, a patient administered tavapadon according to the methods disclosed herein has a UPDRS score decreased by at least about 5% compared to baseline. In certain embodiments, a patient administered tavapadon according to the methods disclosed herein has a UPDRS score decreased by at least about 5%, at least about 10%, at least about 15%, at least about 20%, at least about 25%, at least about 50%, at least about 75%, or at least about 100% compared to baseline. In certain embodiments, a patient administered tavapadon according to the methods disclosed herein has a UPDRS score decreased by about 1% to about 2%, by about 1% to about 5%, by about 1% to about 10%, by about 1% to about 15%, by about 1% to about 20%, by about 1% to about 25%, by about 1% to about 50%, by about 1% to about 75%, by about 1% to about 100%, by about 5% to about 25%, by about 25% to about 50%, or by about 5% to about 10%. In certain embodiments, a patient administered tavapadon according to the methods disclosed herein has a UPDRS score decreased by about 5%, about 10%, about 15%, about 20%, about 25%, about 50%, about 75%, about 100%, or more than 100% compared to baseline.

The methods disclosed herein may be beneficial in treating one or more motor symptoms resembling the motor symptoms that are characteristic of PD. Non-limiting examples of diseases and/or disorders having motor symptoms similar to those associated with PD can include supranuclear palsy (PSP), multiple system atrophy (MSA), Lewy body disease or dementia with Lewy bodies (LBD), corticobasal degeneration (CBD), and corticobasal ganglionic degeneration.

### 6.7 Diagnostic Criteria and PD Disease Stage

Provided herein are various methods for selecting patients for treatment with the methods disclosed herein. Also provided herein are methods of selecting patients with various stages of PD to be treated with the methods disclosed herein. Methods of diagnosing subjects to be treated with a method disclosed herein can include assessing diagnostic criteria and/or disease stage according to any of the methods described in this section. Methods of determining disease stage, as detailed below, are also available for measuring the severity and/or rate of progression of PD. In certain embodiments, one or more criteria described in sections 5.7.1 to 5.7.9 are used for efficacy assessment during the treatment.

### 6.7.1 UKBB Criteria

In certain embodiments, a PD diagnosis may be carried out according to UK Parkinson's Disease Society Brain Bank (UKBB) diagnostic criteria as described in Gibb and Lees, J Neurol Neurosurg Psychiatry. 1988; 51:745-52). There are three steps for applying the UKBB diagnostic criteria to arrive at a diagnosis of PD. In step one (1), the subject must demonstrate bradykinesia and one of the following features: rigidity, 4-6 Hz rest tremor, or postural instability not caused by primary visual, vestibular, cerebellar or proprioceptive dysfunction. For step two (2), all other causes of parkinsonism must be excluded. Specifically, all secondary causes of a parkinsonian syndrome including history of repeated strokes or head injury with stepwise progression of parkinsonian features, history of definite encephalitis, oculogyric crises, neuroleptic treatment at onset of symptoms, familial history, unilateral features after 3 years, supranuclear gaze palsy, cerebellar signs, early severe autonomic involvement or dementia, unexplained Babinski sign, presence of a secondary cause on imaging, negative response to levodopa, and exposure to toxic agents should be excluded. And for step three (3), the subject must present with at least three of the following supportive (prospective) criteria: unilateral onset; rest tremor; progressive disorder; persistent asymmetry primarily affecting side of onset; excellent response (70-100%) to levodopa; severe levodopa-induced chorea (dyskinesia); levodopa response for 5 years or more; and/or clinical course of 10 years or more.

In certain embodiments of the methods provided herein, the subject having more than one affected relative (Step 2 UK Parkinson's Disease Society Brain Bank exclusion criterion) but meeting all other criteria consistent with the UK criteria will be considered to have met eligibility criteria for a diagnosis of PD.

### 6.7.2 Gelb Criteria

In certain embodiments, PD may be diagnosed according to Gelb criteria as described in Gelb et al., Arch Neurol. 1999; 56:33-9. The Gelb criteria characterize symptoms into two groups: Group A, which include symptoms characteristic of PD; and Group B, which include symptoms suggestive of alternative diagnoses. Group A symptoms include: resting tremor; bradykinesia; rigidity; and asymmetric onset. Group B symptoms include: features unusual early in the clinical course; prominent postural instability in the first 3 years after symptom onset; freezing phenomena in the first 3 years; hallucinations unrelated to medications in the first 3 years; dementia preceding motor symptoms or in the first year; supranuclear gaze palsy (other than restriction of upward gaze) or slowing of vertical saccades; severe, symptomatic dysautonomia unrelated to medications; and documentation of a condition known to cause parkinsonism and plausibly connected to the patient's symptoms (such as suitably located focal brain lesions or neuroleptic use within the past 6 months). For a diagnosis of “possible PD” according to Gelb criteria, a subject 1) must demonstrate at least 2 of the 4 symptoms in Group A where at least 1 of these Group A symptoms is tremor or bradykinesia; 2) either demonstrate none of the symptoms from Group B or symptoms have been present for less than 3 years, and none of the features in Group B is present to date; and 3) either substantial and sustained response to levodopa or a dopamine agonist has been documented or the subject has not had an adequate trial of levodopa or dopamine agonist. For a diagnosis of “probable PD” according to Gelb criteria, a subject 1) must demonstrate at least 3 of the 4 features in Group A; 2) either demonstrate none of the symptoms from Group B or symptoms have been present for less than 3 years, and none of the features in Group B is present to date; and 3) a substantial and sustained response to levodopa or a dopamine agonist has been documented. A diagnosis of “definite” PD according to the Gelb criteria requires post-mortem neuropathologic confirmation in subjects with the clinical diagnosis of possible or probable PD.

### 6.7.3 MDS Criteria

In certain embodiments, PD may be diagnosed in a subject according to Movement Disorder Society (MDS) clinical diagnostic criteria as described in Goetz C G et al. Mov Disord. 2008; 15; 23(15):2129-70.

The MDS-UPDRS has four parts:

Part I, nonmotor aspects of experiences of daily living, comprises 13 items, 6 of which are rated by the physician (Part IA) and 7 of which are rated by the patient (Part IB).

Part II, motor aspects of experiences of daily living, comprises 13 items that are rated by the patient. The 13 items in Part II and the 7 items in Part IB constitute the patient questionnaire portion of the MDS-UPDRS.

Part III, motor examination, comprises 18 items that are assessed by the investigator (resulting in 33 scores by location and lateralization). Part I (non-motor experiences of daily living), Part II (motor experiences of daily living), Part III (motor examination) and Part IV (motor complications).

Part IV, motor complications, comprises 6 item (3 items for dyskinesia and 3 items for fluctuation) and requires the physician to use historical and objective information to assess dyskinesia and motor fluctuations.

Each item is rated on a scale from 0 to 4 on which 0=normal, 1=slight, 2=mild, 3=moderate, and 4=severe.

In one embodiment of the methods provided herein, the subject has an MDS-UPDRS Part II score≥2 and Part III score≥10 at the screening visit and at the baseline visit.

### 6.7.4 Hoehn & Yahr Scale

In certain embodiments, the stage of PD progression can be defined according to the Hoehn and Yahr scale according to the methods described in Hoehn and Yahr, Neurology. 1967 May; 17(5):427-42, or alternatively the modified Hoehn and Yahr scale according to Goetz et al., Movement Disorders. 2004 19(9):1020-1028. The Hoehn and Yahr scale uses five stages, from 1 to 5 where on this scale, stages 1 and 2 represent early-stage, 2 and 3 mid-stage, and 4 and 5 advanced-stage PD. The modified Hoehn and Yahr scale includes additional of stages 1.5 and 2.5 to assess the intermediate course of the disease.

In one embodiment, the subject has modified Hoehn and Yahr stage 1, 1.5, or 2. In one embodiment, the subject has modified Hoehn and Yahr stage 2, 2.5, or 3 in the “on” state. In one embodiment, the subject has modified Hoehn and Yahr stage 2, 2.5, or 3 in the “on” state; and a minimum of 21 hours of “off” time on 2 consecutive days.

The symptoms used to characterize the stage of PD for both scales are provided in Table 1.

TABLE 1
Hoehn and Yahr Scales for PD Progression

| Stage | Hoehn and Yahr Scale | Modified Hoehn and Yahr Scale |
|---|---|---|
| 1 | Unilateral involvement only usually | Unilateral involvement only |
| | with minimal or no functional | |
| | disability | |
| 1.5 | — | Unilateral and axial involvement |
| 2 | Bilateral or midline involvement | Bilateral involvement without impairment of |
| | without impairment of balance | balance |
| 2.5 | — | Mild bilateral disease with recovery on pull |
| | | test |
| 3 | Bilateral disease: mild to moderate | Mild to moderate bilateral disease; some |
| | disability with impaired postural | postural instability; physically independent |
| | reflexes; physically independent | |
| 4 | Severely disabling disease; still able | Severe disability; still able to walk or stand |
| | to walk or stand unassisted | unassisted |
| 5 | Confinement to bed or wheelchair | Wheelchair bound or bedridden unless aided |
| | unless aided | |

### 6.7.5 Generic Clinical Global Impression (CGI)

In certain embodiments, the stage of PD progression can be defined according to the generic Clinical Global Impression (CGI). The CGI has two main components, respectively focused on severity (CGIS) and change (CGIC).

The Clinical Global Impression—Severity of Illness (CGI-S) scale is a 7-point scale that requires the clinician to rate the severity of the patient's illness at the time of the assessment relative to the clinician's past experience with patients who have the same diagnosis (Guy W, editor. ECDEU Assessment Manual for Psychopharmacology. Rockville, MD: US Department of Health, Education, and Welfare Public Health Service Alcohol, Drug Abuse, and Mental Health Administration; 1976).

Raters select one response based on the following question:
“Considering your total clinical experience with this particular population, how ill is the patient at this time?”
Scores are: 1=normal, not at all ill; 2=borderline ill; 3=mildly ill; 4=moderately ill; 5=markedly ill; 6=severely ill; and 7=among the most extremely ill patients.

The Clinical Global Impression—Improvement (CGI-I) a 7-point scale that requires the clinician to assess how much the patient's illness has improved or worsened relative to the baseline state at the beginning of the intervention (Guy W, editor. ECDEU Assessment Manual for Psychopharmacology. Rockville, MD: US Department of Health, Education, and Welfare Public Health Service Alcohol, Drug Abuse, and Mental Health Administration; 1976).

Raters select one response based on the following question:
“Compared to your patient's condition at the beginning of treatment, how much has your patient changed?”
Scores are: 1=very much improved; 2=much improved; 3=minimally improved; 4=no change; 5=minimally worse; 6=much worse; and 7=very much worse.

The PGIC is the patient-reported outcome counterpoint to the CGI-I. The qualitative assessment of meaningful change is determined by the patient in response to the question:
“Compared to your condition at the beginning of treatment, how much has your condition changed?”
Scores are: 1=very much improved; 2=much improved; 3=minimally improved; 4=no change; 5=minimally worse; 6=much worse; and 7=very much worse.

### 6.7.6 39-Item Parkinson's Disease Questionnaire

The 39-item Parkinson's Disease Questionnaire (PDQ-39) is the most thoroughly validated and extensively used self-report measure for the assessment of health-related quality of life in people with PD. The questionnaire measures 39 items, which assess 8 domains of health: mobility (10 items), activities of daily living (6 items), emotional well-being (6 items), stigma (4 items), social support (3 items), cognitions (4 items), communication (3 items), and bodily discomfort (3 items) (Peto et al, a review of the development, validation and application of a Parkinson's disease quality of life questionnaire and its associated measures. J Neurol. 1998; 245(Suppl 1):S10-4). Each item is scored on the following scale: 0=never, 1=occasionally, 2=sometimes, 3=often, and 4=always. Items in each subscale and the total scale can be summarized into an index and transformed linearly to a scale from 0 (perfect health as assessed by the measure) to 100 (worst health as assessed by the measure).

### 6.7.7 Schwab and England Activities of Daily Living Scale

The Schwab and England Activities of Daily Living (ADL) scale is a method of assessing a person's ability to perform daily activities in terms of speed and independence through a percentage figure (Schwab and England, 1969). The clinician determines the rating according to the following criteria, with 100% indicating total independence and 0% indicating a state of complete dependence:

100% Completely independent. Able to do all chores without slowness, difficulty or impairment. Essentially normal. Unaware of any difficulty.

90% Completely independent. Able to do all chores with some degree of slowness, difficulty and impairment. May take twice as long. Beginning to be aware of difficulty

80% Completely independent in most chores. Takes twice as long. Conscious of difficulty and slowness.

70% Not completely independent. More difficulty with some chores. Three to four times as long in some. May spend a large part of the day with chores.

60% Some dependency. Can do most chores, but exceedingly slowly and with much effort. Errors; some impossible.

50% More dependent. Help with half, slower, etc. Difficulty with everything.

40% Very dependent. Can assist with all chores, but few alone

30% With effort, now and then does a few chores alone or begins alone. Much help needed

20% Nothing alone. Can be a slight help with some chores. Severe invalid

10% Totally dependent, helpless. Complete invalid.

0% Vegetative functions such as swallowing, bladder and bowel functions are not functioning. Bedridden.

### 6.7.8 EuroQol 5 Dimension 5 Level

The EuroQol 5 Dimension 5 Level (EQ-5D-5L) is a patient-reported outcome that measures health in 5 dimensions. It is a widely used survey instrument for measuring economic preferences for health states, is applicable to a wide variety of health conditions and treatments, and provides a simple descriptive profile and a single index value for: health status (Herdman et al, 2011). The EQ-5D-5L consists of a descriptive system and a visual analog scale (VAS).

The descriptive system comprises 5 dimensions: mobility, self-care, usual activities, pain/discomfort, and anxiety/depression. Each dimension has 5 levels: no problems, slight problems, moderate problems, severe problems, and extreme problems. The patient is asked to indicate his or her health state by ticking the box next to the most appropriate statement in each of the 5 dimensions. This decision results in a 1-digit number that expresses the level that was selected for that dimension. The digits for the 5 dimensions are combined into a 5-digit number that describes the patient's health state.

The EQ-5D-5L VAS records the patient's self-rated health on a vertical VAS on which the endpoints are labeled “the best health quality that you can imagine” and “the worst health quality that you can imagine.” The VAS can be used as a quantitative measure of health outcome that reflects the patient's own judgment.

### 6.7.9 Other Markers of PD

In certain embodiments, PD may be diagnosed by other markers associated with the disease other than clinical presentation of symptoms described herein. In some embodiments, PD can be diagnosed by imaging methods, such as magnetic resonance imaging (MRI), positron emission tomography (PET) scan, and/or dopamine transporter scan (DaT scan). A DaTscan involves injection of a small amount of a radioactive drug that is then measured by a single-photon emission computed tomography scanner (SPECT scanner). The SPECT scanner measures the levels and location of the drug in the brain. The resulting DAT-SPECT scans can be visually categorized by an observer according to predefined visual patterns of dopaminergic degeneration and may be graded as normal (grade 1) or abnormal (grade 2-5), distinguishing an almost normal, symmetrical tracer uptake with a discrete reduction in one or both putamina (grade 2 “eagle wing”), an asymmetric tracer uptake with normal or almost normal uptake in the putamen of one hemisphere and reduced uptake in the contralateral putamen (grade 3 “mixed type”), a posterior-anterior degeneration pattern (grade 4 “egg shape”) and severe degeneration pattern (grade 5 “burst striatum”) (See, e.g., Lloyd et al., Neuroimage Clin. 2018; 20:823-829).

In some embodiments, PD can be diagnosed by a biomarker detected in a fluid or tissue sample collected from the subject suspected of having PD. Non-limiting examples of biomarkers of PD include alpha-synuclein, urate, neurofilament light chain (NfL), amyloid 042, tau, phosphorylated tau (p-tau), cytokines (e.g., IL-1β, IL-6, and TGF-β), and the like.

The following examples and figures are provided to aid the understanding of the present invention, the true scope of which is set forth in the appended claims. It is understood that modifications can be made in the procedures set forth without departing from the spirit of the invention.

### 7. EXAMPLES

The following is a description of various methods and materials used in the studies. They are put forth so as to provide those of ordinary skill in the art with a complete disclosure and description of how to make and use the present invention and are not intended to limit the scope of what the inventors regard as their invention, nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, percentages, etc.), but some experimental errors and deviations should be accounted for.

### 7.1 Example 1: A Phase 3, Double-Blind, Randomized, Placebo-Controlled, Parallel Group, 27-Week Trial to Evaluate the Efficacy, Safety, and Tolerability of Two Fixed Doses of Tavapadon in Early Parkinson's Disease (TEMPO-1 Trial)

This trial is designed to define and confirm the magnitude of efficacy and the risk/benefit profile of tavapadon over the target dose range of 5 to 15 mg once daily (QD).

### Objectives and Endpoints:

The objectives and efficacy, safety, and pharmacokinetic (PK) endpoints of the trial are summarized below.
Primary objective: To assess the efficacy of 2 fixed doses of tavapadon in subjects with early PD
Secondary objective: To assess the safety and tolerability of 2 fixed doses of tavapadon in subjects with early PD

### Primary Efficacy Endpoint

Change from baseline to endpoint in the MDS-UPDRS Parts II and III combined score

### Key Secondary Efficacy Endpoints

Change from baseline to endpoint in the MDS-UPDRS Part II score
Percentage of responders at endpoint, defined as a score of “much improved” or “very much improved” on the PGIC

### Secondary Efficacy Endpoints (All Time Points)

Change from baseline in the MDS-UPDRS Parts II and III combined score
Change from baseline in the MDS-UPDRS Parts I, II, and III combined score
Change from baseline in the MDS-UPDRS Part I, Part II, and Part III individual scores
Change from baseline in the CGI-S score
CGI-I score
PGIC score

### Other Endpoints

Change from baseline in PDQ-39 score
Change from baseline in Schwab and England ADL score
Change from baseline in the EQ-5D-5L index and VAS scores

### Overall Design:

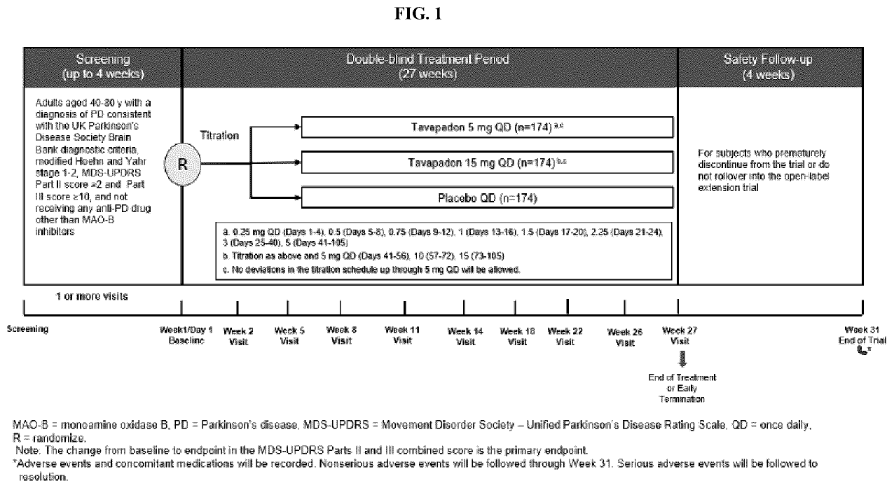

This is a prospective, Phase 3, multicenter, multinational, randomized, double-blind, placebo-controlled, parallel-group, 27-week trial to evaluate the efficacy, safety, tolerability, and PK of 2 fixed doses of tavapadon (5 mg QD and 15 mg QD) in male and female subjects aged 40 to 80 years who have a diagnosis of PD (consistent with the UK Parkinson's Disease Society Brain Bank diagnostic criteria); a modified Hoehn and Yahr stage of 1, 1.5, or 2; a Movement Disorder Society—Unified Parkinson's Disease Rating Scale (MDS-UPDRS) Part II score≥2 and Part III score≥10; and are not receiving antiparkinsonian medication other than monoamine oxidase B (MAO-B) inhibitors. The trial will include a Screening Period (maximum of 4 weeks), a 27-week Treatment Period, and a 4-week Safety Follow-up Period. FIG. 1 provides a schematic for a dose escalation study for tavapadon.

Each subject will participate in the trial for up to approximately 35 weeks, including screening (up to 4 weeks), treatment (27 weeks), and posttreatment safety follow-up (4 weeks). Subjects who complete through Week 27 of the trial may have the opportunity to enter an open-label extension trial. The dosing schedule is provided below:

TABLE 2
Dosing schedule

| | Titration | Target Dose | | |
|---|---|---|---|---|
| Trial Day | Step | Tavapadon 5 mg QD | Tavapadon 15 mg QD | Placebo QD |
| Days 1-4: | Step 1 | 0.25 mg tavapadon QD | 0.25 mg tavapadon QD | Placebo QD |
| Days 5-8: | Step 2 | 0.5 mg tavapadon QD | 0.5 mg tavapadon QD | Placebo QD |
| Days 9-12: | Step 3 | 0.75 mg tavapadon QD | 0.75 mg tavapadon QD | Placebo QD |
| Days 13-16: | Step 4 | 1 mg tavapadon QD | 1 mg tavapadon QD | Placebo QD |
| Days 17-20: | Step 5 | 1.5 mg tavapadon QD | 1.5 mg tavapadon QD | Placebo QD |
| Days 21-24: | Step 6 | 2.25 mg tavapadon QD | 2.25 mg tavapadon QD | Placebo QD |
| Days 25-40: | Step 7 | 3 mg tavapadon QD | 3 mg tavapadon QD | Placebo QD |
| Days 41-56: | Step 8 | 5 mg tavapadon QD | 5 mg tavapadon QD | Placebo QD |
| Days 57-72: | Step 9 | 5 mg tavapadon QD | 10 mg tavapadon QD | Placebo QD |
| Days 73-189: | Step 10 | 5 mg tavapadon QD | 15 mg tavapadon QD | Placebo QD |

QD—once daily

This titration scheme will be used for all subjects who are randomized to tavapadon. Subjects who are randomized to the tavapadon 5 mg QD treatment group will receive this dose from Day 41 until Day 189 (end of Week 27). The dose of tavapadon will continue to be titrated for subjects who are randomized to the tavapadon 15 mg QD treatment group; these subjects will receive 10 mg QD of tavapadon from Day 57 through Day 72 and then 15 mg QD of tavapadon from Day 73 until Day 189 (end of Week 27).

Titration of tavapadon should be guided by absence of tolerability issues that are reported as AEs and that are of sufficient severity resulting in significant dysfunction or distress to the subject. Subjects who cannot achieve or tolerate the dose at Step 8 (5 mg QD) or Step 9 (10 mg QD) must be discontinued from the trial.

Starting on Day 74, subjects who achieve but do not tolerate the dose at Step 10 may go back to the dose at Step 9. Subjects may remain at the Step 9 dose or will be permitted a one-time rechallenge back to the Step 10 dose if needed for symptomatic control. Subjects must receive the Step 9 dose for at least 7 days before a rechallenge to the Step 10 dose is attempted. Subjects who are unable to tolerate the Step 10 dose upon rechallenge will be discontinued from the trial. The first dose of IMP will be taken at the trial site at the Baseline Visit; all other doses will be taken on an outpatient basis. Assessments will be conducted in the clinic, ˜2 to ˜6 hours after the subjects have taken their daily dose of IMP (at home), at the end of Weeks 2, 5, 8, 11, 14, 18, 22, 26, and 27. Blood samples for PK will be collected after administration of the first dose on Day 1 (˜1 hour after dosing) and at the Weeks 5, 11, 14, 22, and 27 clinic visits.

Concentration-QT Substudy: A substudy to evaluate the effect of tavapadon on the QT interval using concentration-QT modelling is also included in this trial. Matched triplicate electrocardiograms (ECGs) and plasma tavapadon concentrations that are collected at preselected sites will be used to characterize the effects of tavapadon on the QT interval.

Number of Subjects: A total of 522 subjects are planned to be randomized into 3 treatment groups (174 subjects per treatment group).

Key Entry Criteria: Male and female subjects aged 40 to 80 years who have a diagnosis of PD (consistent with the UK Parkinson's Disease Society Brain Bank diagnostic criteria); a modified Hoehn and Yahr stage of 1, 1.5, or 2; an MDS-UPDRS Part II score≥2 and Part III score≥10; and are not receiving antiparkinsonian medication other than MAO-B inhibitors will be enrolled.

Intervention Groups, Trial Treatments, and Duration: Subjects will be randomized in a 1:1:1 ratio to receive tavapadon 5 mg QD, tavapadon 15 mg QD, or placebo QD. The dose of tavapadon will be titrated to a target dose using a fixed titration scheme.

Randomization will be stratified by concurrent use of allowed PD medications (MAO-B inhibitors) during the Treatment Period. The planned duration of treatment is 27 weeks, including the Titration, Adjustment, and Maintenance Phases. The investigational medicinal products (IMPs) will be taken orally.

### Statistical Methods

Sample Size Estimation: A sample size of 174 subjects per group (total, 522 subjects) will provide at least 90% power to detect a change in the primary outcome measure (change from baseline in the MDS-UPDRS Parts II and III combined score) of 4 points, with a standard deviation of 9, 2-sided alpha level=0.05, and assuming a 27% dropout rate.

Efficacy Analyses: The efficacy endpoints will be assessed for each of the 2 dose levels of tavapadon versus placebo. A hierarchical testing procedure with a prespecified order for the primary and key secondary endpoints will be used to control the Type I error rate at the 0.05 level (2-sided).

The primary efficacy endpoint (the change from baseline to endpoint in the MDS-UPDRS Parts II and III combined score) will be analyzed using a Mixed Model for Repeated Measures (MMRM). The change from baseline at each postbaseline timepoint will be included in the MMRM as repeated measures. The baseline score will be included as a covariate, and treatment group, visit, the interaction between treatment group and visit, and the stratification factor (concurrent use of MAO-B inhibitor; yes/no) will be included as fixed factors. An unstructured covariance structure will be utilized. The difference between each tavapadon dose level and placebo at endpoint will be estimated based on the least square means (LSMeans) from the MMIRM. A hypothetical strategy will be used to address intercurrent events (ICEs) of potential death, treatment discontinuations, missed visits/assessments, and start of prohibited concomitant medications. The data after treatment discontinuations or after the start of prohibited concomitant medications will be censored under the hypothetical strategy. The missing values are assumed to be missing at random (MAR) in the primary analysis, including missing due to COVID-19 control measures. Sensitivity analyses will be performed to assess the impact of deviation from MAR assumptions on the analysis results.

The key secondary endpoint of the change from baseline to endpoint in the MDS-UPDRS Part II score will be analyzed using an MMRM analysis similar to the analysis of the primary endpoint.

The key secondary endpoint of the percentage of responders at endpoint, defined as a score of “much improved” or “very much improved” on the Patient Global Impression of Change (PGIC), will be analyzed using the SAS® GLIMMIX procedure for binomial data with logit link. This generalized linear mixed model analysis will include response data from each postbaseline visit as repeated measures with treatment group, visit, and interaction between treatment group and visit as fixed effects. An unstructured covariance structure will be used for the repeated measures. If the model fails to converge with the unstructured covariance structure, the heterogeneous Toeplitz structure or further reduced covariance structure may be used.

Other secondary endpoints (as listed above) will be analyzed similarly to the primary efficacy endpoint if the data are of a continuous nature, and similarly to the key secondary endpoint of percentage of responders on the PGIC at endpoint if the data are of ordered categorical nature.

Safety Analyses: Treatment-emergent adverse events (TEAEs) will be coded according to the Medical Dictionary for Regulatory Activities (MedDRA) and summarized by treatment group, system organ class, and preferred term. Additional summaries by seriousness, severity, relationship to IMP, and dose at the time of onset will also be prepared. Other safety endpoints will be summarized with descriptive statistics. Abuse potential will be assessed through active monitoring of events subject to additional monitoring (ESAM; i.e., TEAEs related to abuse potential and TEAEs related to medication handling irregularities [MHIs]).

Inclusion Criteria: Subjects are eligible to be included in the trial only if all of the following criteria apply:

### General and Administrative:

1. Male and female subjects aged 40 to 80 years, inclusive, at the time of signing the ICF.
2. Sexually active men or women of childbearing potential must agree to use acceptable (at minimum) or highly effective birth control, or remain abstinent during the trial and for 4 weeks after the last dose of trial treatment.
3. Subjects who are capable of giving signed informed consent, which includes compliance with the requirements and restrictions listed in the ICF and in this protocol.
4. Subjects who are able, in the opinion of the investigator, to understand the nature of the trial and comply with protocol requirements, including the prescribed dosage regimens, scheduled visits, laboratory tests, and other trial procedures.

### Parkinson's Disease Diagnosis

5. Subjects with a diagnosis of PD that is consistent with the UK Parkinson's Disease Society Brain Bank diagnostic criteria.
   Note: Subjects having more than one affected relative (Step 2 UK Parkinson's Disease Society Brain Bank exclusion criterion) but meeting all other criteria consistent with the UK criteria will be considered to have met eligibility criteria for a diagnosis of PD.
   Note: Motor asymmetry may or may not be present at the time of Screening, and is not an explicit requirement for inclusion, as long as the subject meets the UK Parkinson's Disease Society Brain Bank diagnostic criteria based on medical history or physical examination.
6. Subjects with modified Hoehn and Yahr stage 1, 1.5, or 2.
7. Subjects with disease duration (from time of diagnosis) of <3 years and disease progression in the 3 years before signing the ICF.
8. Subjects with an MDS-UPDRS Part II score≥2 and Part III score≥10 at the Screening Visit and at the Baseline Visit.
9. Subjects with early PD who, in the opinion of the investigator, require pharmacologic intervention for disease management.

### Concomitant Parkinson's Disease Medications

10. Subjects who are treatment naïve or have a history of prior incidental treatment with dopaminergic agents (including L-Dopa and dopamine receptor agonist medications) for <3 months in total but not within 2 months of the Baseline Visit. Prior and concurrent use of MAO-B inhibitors is permitted if use was initiated >90 days before the Baseline Visit and the dosage will remain stable for the duration of the trial (i.e., no change in the MAO-B inhibitor dose is permitted during the trial).
11. Subjects who are willing and able to refrain from any PD medications that are not permitted by the protocol (including dopaminergic agents) throughout participation in the trial.

Abbreviations: ICF=informed consent form, IMP=investigational medicinal product, L-Dopa=levodopa, MAO-B=monoamine oxidase B, MDS-UPDRS=Movement Disorder Society-Unified Parkinson's Disease Rating Scale, PD=Parkinson's disease.

### Exclusion Criteria

Subjects are excluded from the trial if any of the following criteria apply:

### Parkinson's Disease Diagnosis

1. Subjects with a history or clinical features consistent with essential tremor, atypical or secondary parkinsonian syndrome (including, but not limited to, progressive supranuclear palsy, multiple system atrophy, cortico-basal degeneration, or drug-induced or poststroke parkinsonism).
2. Subjects with a history of nonresponse or insufficient response to L-Dopa or 2 or more other antiparkinsonian drugs at therapeutic dosages.
3. Subjects who have had previous surgical intervention (e.g., deep brain stimulation) for PD or for whom such a procedure is planned or anticipated during the trial period.

### Medical History

4. Subjects with an acute or chronic, clinically significant medical or psychiatric condition, cognitive impairment, or laboratory abnormality that might increase the risk associated with trial participation or administration of trial treatment or interfere with the interpretation of the trial results or that, in the judgment of the investigator, would make the subject inappropriate for entry into this trial.
   Medical conditions that are minor or well controlled may be considered acceptable if the condition does not expose the subject to an undue risk of a significant AE or interfere with the assessments of safety or efficacy during the course of the trial. Subjects with symptoms of anxiety or depression that are not debilitating and that are stable or adequately controlled with non-prohibited medication are considered acceptable. The medical monitor should be contacted in any instance where the investigator is uncertain regarding the stability of a subject's medical conditions(s) and the potential impact of the condition(s) on trial participation.
5. Subjects with a history or current diagnosis of a clinically significant impulse control disorder (Disruptive, Impulse Control, and Conduct Disorder per DSM-5) (American Psychiatric Association, 2013).
6. Subjects with the presence of or history of brain tumor, hospitalization for severe head trauma, epilepsy (as defined by the International League Against Epilepsy), or seizures.
7. Subjects with a history of psychosis or hallucinations within the previous 12 months based on medical records or subject/caregiver feedback.
8. Subjects who answer “yes” on the C-SSRS Suicidal Ideation Item 4 or Item 5 (Active Suicidal Ideation with Some Intent to Act, Without Specific Plan, or Active Suicidal Ideation with Specific Plan and Intent) and whose most recent episode meeting the criteria for C-SSRS Item or Item 5 occurred within the last 6 months, OR
   Subjects who answer “yes” on any of the 5 C-SSRS Suicidal Behavior Items (actual attempt, interrupted attempt, aborted attempt, preparatory acts, or behavior) and whose most recent episode meeting the criteria for any of these 5 C-SSRS Suicidal Behavior Items occurred within the last 2 years, OR
   Subjects who, in the opinion of the investigator, present a serious risk of suicide.
9. Subjects with substance abuse or dependence disorder, including alcohol, benzodiazepines, and opioids, but excluding nicotine, within the past 6 months (180 days).
10. Subjects with dementia or cognitive impairment that, in the judgement of the investigator, would exclude the subject from understanding the ICF or participating in the trial.
11. Subjects with any condition that could possibly affect drug absorption, including bowel resections, bariatric weight loss surgery, or gastrectomy (this does not include gastric banding).
12. Subjects who have a positive result for HIV antibodies, HbsAg, or HCV antibodies at screening.
   Note: Subjects who were previously infected with hepatitis C but have been successfully treated (defined as a sustained virologic response or undetectable hepatitis C viral RNA levels at 12 or more weeks after the end of treatment) may be enrolled after discussion with the medical monitor.
13. Subjects with a history of malignancy other than:
   Non-metastatic basal or squamous cell carcinoma of the skin or carcinoma in situ that was surgically removed in total>1 year before signing the ICF and had not recurred
   Another type of malignancy that had been in remission for ≥5 years before signing the ICF and had not recurred.
14. Subjects with a history of myocardial infarction with residual atrial, nodal, or ventricular arrhythmias that are not controlled with medical and/or surgical intervention; second- or third-degree atrioventricular block; sick sinus syndrome; severe or unstable angina; or congestive heart failure within the last 12 months. A recent (≤12 months) history of myocardial infarction with secondary arrhythmias is exclusionary regardless of the therapeutic control.
15. Subjects with a history of neuroleptic malignant syndrome.
16. Female subjects who are breastfeeding and/or who have a positive pregnancy test result prior to receiving IMP.

### Prior or Concomitant Medications

17. Subjects who are currently receiving moderate or strong CYP3A4 inducers or CYP3A4 inhibitors (except for topical administration).
18. Subjects with a positive urine drug screen for illicit drugs are excluded and may not be retested or rescreened. Subjects with a positive urine drug screen resulting from use of marijuana (any THC-containing product), prescription, or over-the-counter medications or products that, in the investigator's documented opinion, do not signal a clinical condition that would impact the safety of the subject or interpretation of the trial results may continue evaluation for the trial following consultation and approval by the medical monitor.
19. Subjects who are using prohibited medications prior to randomization or who would be likely to require the use of prohibited concomitant medications during the trial.

### Screening Assessments

20. Subjects with a MoCA score<26.
21. Subjects with a supine blood pressure≥160 mmHg (systolic) or ≥100 mmHg (diastolic) at screening. The average of two supine measurements will be used to assess eligibility.
22. Subjects with clinically significant orthostatic hypotension (e.g., syncope).
23. Subjects with a 12-lead ECG demonstrating a QTcF interval>450 msec
At screening:
   If the QTcF interval is >450 msec on the machine reading, the ECG should be repeated with 2 additional recordings. Based on the QTcF intervals that are reported by the central service, a subject will be excluded if the QTcF interval is >450 msec on 2 or more of the 3 ECG recordings, unless due to ventricular pacing.
At baseline:
   If the QTcF interval is >450 msec on the machine readings, consult the medical monitor to determine whether the subject remains eligible to be randomized while awaiting the readings from the central service.
24. Subjects with moderate or severe renal impairment (creatinine clearance as estimated by Cockcroft-Gault formula<30 mL/min or on dialysis).
25. Subjects with any of the following abnormalities in clinical laboratory tests at the Screening Visit, as assessed by the central laboratory and confirmed by a single repeat measurement, if deemed necessary:
   AST or ALT≥3×ULN.
   Total bilirubin≥1.5×ULN. Subjects with a history of Gilbert's syndrome may be eligible provided they have a value<ULN for direct bilirubin.
Subjects with other abnormal laboratory test results, vital sign results, or ECG findings unless, in the judgment of the investigator, the findings are not medically significant and would not impact the safety of the subjects or the interpretation of the trial results. The medical monitor should be contacted to discuss individual cases, as needed. Tests with exclusionary results should be repeated to ensure reproducibility of the abnormality before excluding a subject based on the criteria provided in the protocol. For medically significant or exclusory abnormal ECGs results, 2 additional ECG recordings should be collected, to ensure reproducibility of the abnormality, and the 3 ECG recordings read by the central service to confirm the abnormality before excluding a subject.

### Other

26. Subjects who previously participated in any tavapadon trial, including this trial, and received IMP.
27. Subjects who received treatment with any other investigational drug within 60 days before signing the ICF.
28. Any subject who, in the opinion of the sponsor, investigator, or medical monitor, should not participate in the trial.

Abbreviations: AE=adverse event, ALT=alanine aminotransferase, AST=aspartate aminotransferase, C-SSRS=Columbia-Suicide Severity Rating Scale, CYP=cytochrome P450, DSM-5=Diagnostic and Statistical Manual of Mental Disorders, 5th Edition, ECG=electrocardiogram, HbsAg=hepatitis B surface antigen, HCV=hepatitis C virus, HIV=human immunodeficiency virus, ICF=informed consent form, IMP=investigational medicinal product, L-Dopa=levodopa, MoCA=Montreal Cognitive Assessment, PD=Parkinson's disease, QTcF=QT interval as corrected for heart rate by Fridericia's formula, THC=tetrahydrocannabinol, ULN=upper limit of normal.

### Results

### Efficacy

The primary and key secondary endpoints for the two doses was tested in sequential order at 2-sided α=0.05 in the following order:
1. Tavapadon 15 mg QD vs. placebo on change from baseline (CFB) to Week 26 in MDS-UPDRS Pars II+III Combined Score
2. Tavapadon 5 mg QD vs. placebo on CFB to Week 26 in MDS-UPDRS Pars II+III Combined Score
3. Tavapadon 15 mg QD vs. CFB to Week 26 in placebo on MDS-UPDRS Pars II Score
4. Tavapadon 15 mg QD vs. placebo on Patient Global Impression of Change (PGIC) responder at Week 26
5. Tavapadon 5 mg QD vs. placebo on CFB to Week 26 in MDS-UPDRS Pars II Score
6. Tavapadon 5 mg QD vs. placebo on PGIC responder at Week 26

MDS-UPDRS Part II is based on the patient's assessments of motor aspects of experiences of daily living and contains 13 items with total score ranges from 0 to 52. Part III of the MDS-UPDRS is the motor evaluation comprising 18 items that are assessed by the investigator (resulting in 33 scores by location and lateralization). Part III score ranges from 0- to 132. Parts II+III combined score ranges from 0 to 184, with higher score indicating increasing severity of motor symptoms. A negative change from baseline represents an improvement in motor function.

The PGIC is a patient-reported outcome in a 7-point scale to rate how much the patient's illness has improved or worsened relative to the baseline state with scores from 1 to 7 with 1 being very much improved and 7 being very much worse. The PGIC responder is defined as a score of “much improved” or “very much improved” of a given assessment.

The primary efficacy assessment timepoint was specified apriori as Week 26 of the trial.

Both tavapadon 15 mg and 5 mg QD demonstrated a statistically significant and clinically meaningful improvement over placebo in the primary endpoint of MDS-UPDRS Parts II+III Combined Score. The hypothesis testing sequence continued unbroken to show a statistically significant improvement over placebo (with multiplicity taken into account) of both tavapadon 15 mg and 5 mg QD in the key secondary efficacy endpoints of MDS-UPDRS Parts II and PGIC Responder (Table 3 below).

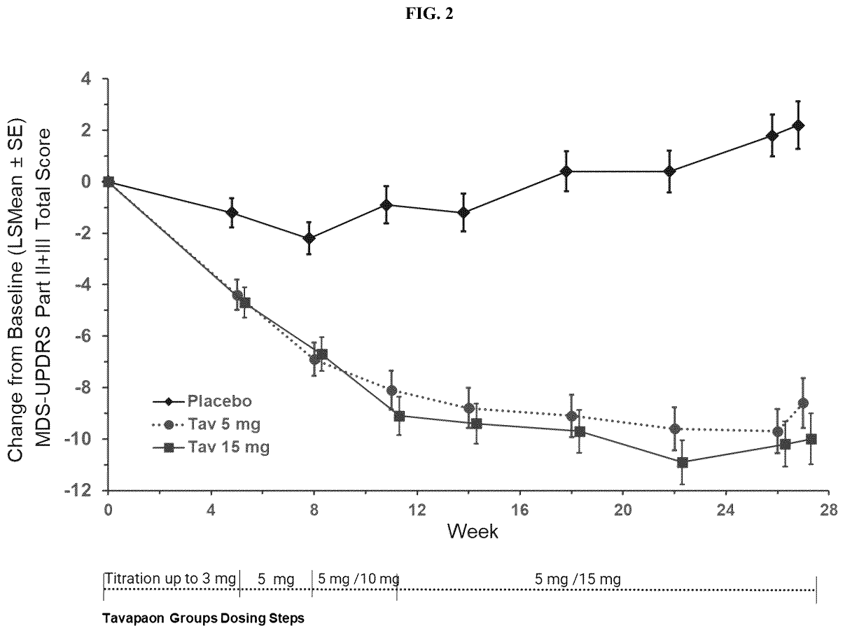

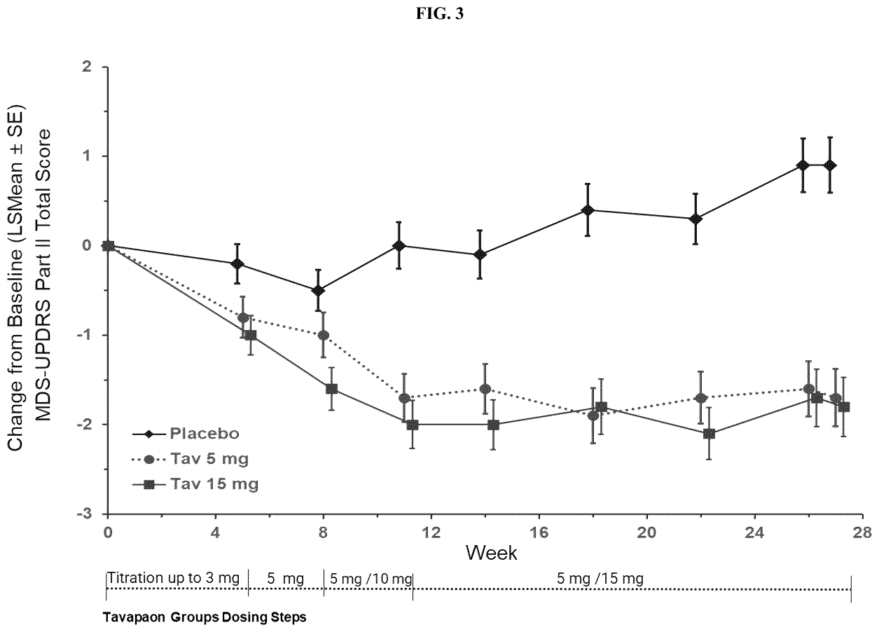

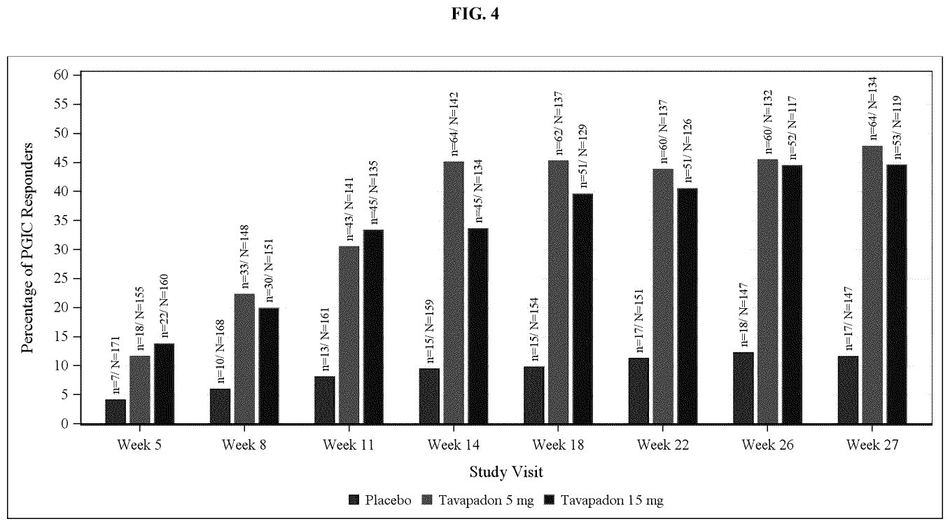

The time course of MDS-UPDRS Part I+III, MDS-UPDRS Part II, and PGIC responder showed a separation from placebo early and sustained for that both doses of tavapadon groups (FIGS. 2, 3, and 4) reaching nominal statistical significance as early as Week 5. Similar results were also observed on MDS-UPDRS Part III. At Week 26, the LSMD (95% CI) versus placebo of was −9.4 (−11.2, −7.6) with nominal p-value<0.0001 and −9.0 (−10.8, −7.3) with nominal p-value<0.0001 for tavapadon 15 mg QD and <mg QD, respectively.

TABLE 3
Summary of Primary and Key Secondary Endpoints - mITT Population

| | | | |
|---|---|---|---|
| | | Tavapadon | Tavapadon |
| | Placebo | 5 mg QD | 15 mg QD |
| Key Efficacy Endpoint | (N = 174) | (N = 174) | (N = 172) |

Primary: Change from Baseline to Week 26 in
MDS-UPDRS Parts II + III Combined Score

| | | | | | | |
|---|---|---|---|---|---|---|
| Baseline (SD) | 32.0 | (9.96) | 31.4 | (10.86) | 32.0 | (11.61) |
| LS Mean (95% CI) | 1.8 | (0.2, 3.4) | −9.7 | (−11.3, −8.0) | −10.2 | (−12.0, −8.5) |

| | | | | | |
|---|---|---|---|---|---|
| LS Mean Diff vs Placebo (95% CI) | — | −11.5 | (−13.8, −9.2) | −12.1 | (−14.4, −9.8) |

| | | | |
|---|---|---|---|
| P-value of Difference | — | <0.0001 | <0.0001 |
| Cohen's D Effect Size | — | 1.14 | 1.20 |

Key Secondary 1: Change from Baseline
to Week 26 in MDS-UPDRS Parts II Score

| | | | | | | |
|---|---|---|---|---|---|---|
| Baseline (SD) | 7.4 | (3.83) | 7.1 | (4.00) | 7.7 | (4.26) |
| LS Mean (95% CI) | 0.9 | (0.3, 1.5) | −1.6 | (−2.2, −1.0) | −1.7 | (−2.4, −1.1) |

| | | | | | |
|---|---|---|---|---|---|
| LS Mean Diff vs Placebo (95% CI) | — | −2.5 | (−3.3, −1.7) | −2.6 | (−3.4, −1.7) |

| | | | |
|---|---|---|---|
| P-value of Difference | — | <0.0001 | <0.0001 |
| Cohen's D Effect Size | — | 0.68 | 0.70 |

Key Secondary 2: PGIC Response of “much improved”
or “very much improved” at Week 26

| | | | | | |
|---|---|---|---|---|---|
| Percent (n/N) of Responders | 12.2% (18/147) | 45.5% | (60/132) | 44.4% | (52/117) |
| Odds Ratio (95% CI) | — | 6.15 | (3.34, 11.32) | 5.97 | (3.22, 11.06) |

| | | | |
|---|---|---|---|
| P-value | — | <0.0001 | <0.0001 |

### Safety

Tavapadon 5 mg QD and 15 mg QD were generally safe and tolerated. An increase in treatment-emerging adverse events as compared to placebo was observed in both tavapadon dose groups without clear dose response (Table 4). Most adverse events were mild or moderate. Treatment-emergent SAEs were reported at relatively low rate with most of the events reported by 1 or 2 patients. There were 2 deaths in the placebo group due to COVID-19 infection and 1 death in tavapadon 5 mg QD group with cause unknown.

TABLE 4
Overall Summary of Adverse Events - FAS

| | | | Tavapadon 5 | | Tavapadon 15 | |
|---|---|---|---|---|---|---|
| | Placebo | | mg QD | | mg QD | |
| | (N = 175) | | (N = 177) | | (N = 177) | |
| | n (%) | | n (%) | | n (%) | |
| Treatment-emergent adverse event | 100 | (57.1%) | 142 | (80.2%) | 139 | (78.5%) |
| (TEAE) | | | | | | |
| Mild | 55 | (31.4%) | 73 | (41.2%) | 54 | (30.5%) |
| Moderate | 34 | (19.4%) | 62 | (35.0%) | 68 | (38.4%) |
| Severe | 11 | (6.3%) | 7 | (4.0%) | 17 | (9.6%) |
| TEAE related to IMP | 34 | (19.4%) | 90 | (50.8%) | 99 | (55.9%) |
| Treatment-emergent serious | 11 | (6.3%) | 4 | (2.3%) | 10 | (5.6%) |
| adverse event (TESAE) | | | | | | |
| TEAE leading to discontinuation | 7 | (4.0%) | 29 | (16.4%) | 35 | (19.8%) |
| of treatment | | | | | | |

| | | | | | |
|---|---|---|---|---|---|
| Death | 2 | (1.1%) | 1 | (0.6%) | 0 |

The most common TEAEs in the tavapadon groups that were reported at a substantially higher frequency than the placebo group were nausea, headache, dizziness, fatigue, dysgeusia, dyspepsia, vomiting, dry mouth, and orthostatic hypotension. However, most of the common TEAEs occurred in the first 72 days of the treatment period. The frequency of events decreased considerably afterwards.

The rate of TESAEs was lowest in the tavapadon 5 mg QD group (2.3%) and comparable between placebo (6.3%) and tavapadon 15 mg QD (5.6%) treatment groups. TESAEs reported by more than 1 subject include pneumonia, COVID-19, COVID-19 pneumonia and angina unstable.

Placebo (11 subjects reported 15 SAEs): COVID-19 (2 subjects); COVID-19 and COVID-19 pneumonia; fall, head injury and femur fracture (left, then right); COVID-19 pneumonia; pneumonia; inguinal hernia; atrial fibrillation; prostate cancer; basal cell carcinoma; renal cell carcinoma.

Tavapadon 5 mg QD (4 subjects reported 1 SAE each): prostatomegaly; death; angina unstable; constipation.

Tavapadon 15 mg QD (10 subjects reported 12 SAEs): pneumonia and tracheobronchitis; hallucination, tactile; myocardial infarction; angina unstable; transient ischaemic attack (2 events in same subject); peripheral swelling; coronary artery stenosis; pneumonia; radius fracture; thrombosis.

TEAEs leading to treatment discontinuation occurred more frequently in the tavapadon groups: 7 (4.0%), 29 (16.4%) and 35 (19.8%) in placebo, tavapadon 5 mg QD and tavapadon 15 mg QD respectively. The most frequent events leading to discontinuation of treatment were nausea, headache, and dizziness, consistent with the most reported TEAEs. The frequency decreased considerably after Day 72.

Treatment-emergent AESIs without leading to treatment discontinuation were reported at low rates: 1 (0.6%), 4 (2.3%) and 1 (0.6%) in placebo, tavapadon 5 mg QD and tavapadon 15 mg QD respectively.

### Potentially Clinically Significant Lab and Vital Signs Findings

No safety trends were observed in labs, ECGs or C-SSRS. A decrease from baseline in supine and standing systolic blood pressure (SBP) was observed in both tavapadon dose groups and the mean decrease was approximately 11 to 13 mmHg from Week 11 onward. However, a low rate of cases of SBP<90 mmHg based on the lowest measurement result of the 27-week period were reported: 0 (0.0%), 2 (1.1%) and 5 (2.8%) in placebo, tavapadon 5 mg QD and tavapadon 15 mg QD, respectively. Similarly, a decrease from baseline in supine and standing diastolic blood pressure (DBP) was observed in both tavapadon dose groups and the mean decrease was approximately 6 to 7 mmHg from Week 11 onward. However, a low rate of cases of DBP<50 mmHg based on the lowest measurement result of the 27-week period were reported: 0 (0.0%), 3 (1.7%) and 5 (2.8%) in placebo, tavapadon 5 mg QD and tavapadon 15 mg QD, respectively. No apparent imbalance versus placebo was observed in the orthostatic blood pressures as measured at clinical visits.

### 7.2 Example 2: A Phase 3, Double-Blind, Randomized, Placebo-Controlled, Parallel Group, Flexible-Dose, 27-Week Trial to Evaluate the Efficacy, Safety, and Tolerability of Tavapadon in Early Parkinson's Disease (TEMPO-2 Trial)

This trial is designed to evaluate the efficacy, safety, tolerability, and PK of flexible doses of tavapadon (5 to 15 mg QD) in male and female subjects aged 40 to 80 years who have a diagnosis of PD (consistent with the UK Parkinson's Disease Society Brain Bank diagnostic criteria); a modified Hoehn and Yahr stage of 1, 1.5, or 2; a Movement Disorder Society—Unified Parkinson's Disease Rating Scale (MDS-UPDRS) Part II score≥2 and Part III score≥10; and are not receiving antiparkinsonian medication other than monoamine oxidase B (MAO-B) inhibitors. The trial will include a Screening Period (maximum of 4 weeks), a 27-week Treatment Period, a 10-day Safety/Withdrawal Assessment Period to assess potential withdrawal symptoms after discontinuation of the investigational medicinal product (IMP), and a 20-day Safety Follow-up Period.

### Objectives and Endpoints:

The objectives and efficacy, safety, and pharmacokinetic (PK) endpoints of the trial are summarized below.
Primary objective: To assess the efficacy of tavapadon in subjects with early PD
Secondary objective: To assess the safety and tolerability of tavapadon in subjects with early PD
Pharmacokinetic: To evaluate the PK of tavapadon in this population

### Primary Efficacy Endpoint

Change from baseline to endpoint in the MDS-UPDRS Parts II and III combined score

### Key Secondary Efficacy Endpoints

Change from baseline to endpoint in the MDS-UPDRS Parts II and III combined score Percentage of responders at endpoint, defined as a score of “much improved” or “very much improved” on the PGIC

### Secondary Efficacy Endpoints (All Time Points)

Change from baseline in the MDS-UPDRS Parts II and III combined score
Change from baseline in the MDS-UPDRS Parts I, II and III combined score
Change from baseline in the MDS-UPDRS Parts I, II and III individual scores
Change from baseline in the CGI-S score
CGI-I score
PGIC score

### Other Endpoints

Change from baseline in PDQ-39 score
Change from baseline in Schwab and England ADL score
Change from baseline in the EQ-5D-5L index and VAS scores

### Overall Design:

This is a prospective, Phase 3, multicenter, multinational randomized, double-blind, placebo-controlled, parallel-group, 27-week trial to evaluate the efficacy, safety, tolerability, and PK of flexible doses of tavapadon (5 to 15 mg QD) in male and female subjects aged 40 to 80 years who have a diagnosis of PD (consistent with the UK Parkinson's Disease Society Brain Bank diagnostic criteria); a modified Hoehn and Yahr stage of 1, 1.5, or 2; a Movement Disorder Society—Unified Parkinson's Disease Rating Scale (MDS-UPDRS) Part II score≥2 and Part III score≥10; and are not receiving antiparkinsonian medication other than monoamine oxidase B (MAO-B) inhibitors. The trial will include a Screening Period (maximum of 4 weeks), a 27-week Treatment Period, a 10-day Safety/Withdrawal Assessment Period to assess potential withdrawal symptoms after discontinuation of the investigational medicinal product (IMP), and a 20-day Safety Follow-up Period.

Each subject will participate in the trial for up to approximately 35 weeks. Subjects who complete through Week 27 of the trial and the 10-day Safety/Withdrawal Assessment Period may have the opportunity to enter an open-label extension trial (Protocol CVL-751-PD-004). Subjects who are ineligible or elect not to enter the open-label trial will complete the 10-day Safety/Withdrawal Assessment Period and the 20-day Safety Follow-up period.

The dosing schedule is provided below in Table 5:

TABLE 5
Dosing schedule

| | | |
|---|---|---|
| | Titration | |
| Trial Day | Step | Blinded Treatment Assignment |

Blinded Dose Titration Phase^{a}

| | | |
|---|---|---|
| Days 1-4: | Step 1 | 0.25 mg tavapadon or placebo QD |
| Days 5-8: | Step 2 | 0.5 mg tavapadon or placebo QD |
| Days 9-12: | Step 3 | 0.75 mg tavapadon or placebo QD |
| Days 13-16: | Step 4 | 1 mg tavapadon or placebo QD |
| Days 17-20: | Step 5 | 1.5 mg tavapadon or placebo QD |
| Days 21-24: | Step 6 | 2.25 mg tavapadon or placebo QD |
| Days 25-40: | Step 7 | 3 mg tavapadon or placebo QD |
| Days 41-56: | Step 8 | 5 mg tavapadon or placebo QD |

Blinded Dose Adjustment Phase^{b}

| | | |
|---|---|---|
| Days 57-72: | Step 9 | 10 mg tavapadon or placebo QD |
| Days 73-189: | Step 10 | 15 mg tavapadon or placebo QD |

Blinded Maintenance Phase^{c}

| | | |
|---|---|---|
| Days 105-189 | Step 11 | Maximum tolerated tavapadon |
| | | dose (5-15 mg) or placebo QD |

QD—once daily
^{a}No deviations in the titration schedule up through 5 mg QD (Step 8) will be allowed.
^{b}The dose will be adjusted over the range of 5 to 15 mg (Step 9 and Step 10) based on individual subject tolerability.
^{c}Subjects will receive the highest tolerated dose level that is achieved during the Dose Adjustment Phase. No adjustment in the tavapadon dose will be allowed during the Maintenance Phase.

Titration of tavapadon should be guided by absence of tolerability issues that are reported as AEs and that are of sufficient severity resulting in significant dysfunction or distress to the subject. Any questions regarding tolerability issues and titration should be directed to the medical monitor before adjustments are initiated. The dose of tavapadon will be gradually titrated to the Step 10 dose (15 mg QD) in all subjects, as shown Table 5, unless prevented by intolerance.

Subjects who are unable to achieve or tolerate the Step 10 dose (15 mg QD) may receive the Step 9 dose (10 mg QD). Subjects who cannot achieve or tolerate the Step 9 dose (10 mg QD) may receive the Step 8 dose (5 mg QD). Subjects who cannot achieve or tolerate the Step 8 dose (5 mg QD) will be discontinued from the trial. Subjects who require a dose reduction may be rechallenged with a higher dose to address symptomatic needs after at least 7 days at the lower dose during the Dose Adjustment Phase.

Subjects will receive the highest tolerated dose level that is achieved during the Dose Adjustment Phase for the duration of the subsequent 13-week Maintenance Phase. Adjustments in the dose of tavapadon will not be allowed during the Maintenance Phase. Subjects who cannot tolerate their maintenance dose will be discontinued from the trial.

The first dose of IMP will be taken at the trial site at the Baseline Visit; all other doses will be taken on an outpatient basis. Assessments will be conducted in the clinic at the end of Weeks 2, 5, 8, 11, 14, 18, 22, 26, 27, and 29. During the Treatment Period, the in-clinic assessments will occur ˜2 to ˜6 hours after the subjects have taken their daily dose of IMP (at home). Blood samples for PK will be collected after administration of the first dose on Day 1 (˜1 hour after dosing) and at the Weeks 5, 11, 14, 22, 27, and 29 clinic visits.

Number of Subjects: A total of 296 subjects are planned to be randomized into 2 treatment groups (148 subjects per treatment group).

Key Entry Criteria: Male and female subjects aged 40 to 80 years who have a diagnosis of PD (consistent with the UK Parkinson's Disease Society Brain Bank diagnostic criteria); a modified Hoehn and Yahr stage of 1, 1.5, or 2; an MDS-UPDRS Part II score≥2 and Part III score≥10; and are not receiving antiparkinsonian medication other than MAO-B inhibitors will be enrolled.

Intervention Groups, Trial Treatments, and Duration: Subjects will be randomized in a 1:1 ratio to receive tavapadon 5 to 15 mg QD or placebo QD. Randomization will be stratified by concurrent use MAO-B inhibitors during the Treatment Period. The planned duration of treatment is 27 weeks, including the Dose Titration, Dose Adjustment, and Maintenance Phases. The IMPs will be taken orally.

### Statistical Methods

Sample Size Estimation: A sample size of 148 subjects per group (total, 296 subjects) will provide at least 90% power to detect a change in the primary outcome measure (change from baseline in the MDS-UPDRS Parts II and III combined score) of 4 points, with a standard deviation of 9, 2-sided alpha level=0.05, and assuming a 27% dropout rate.

In the event of higher than anticipated early terminations due to COVID-19 or other reasons, enrollment may extend in order to maintain the planned statistical power

Efficacy Analyses: Each primary and secondary endpoint will include the comparison of tavapadon (5 to 15 mg) QD versus placebo. To avoid inflation of Type I error rate that may occur due to multiple testing of the 2 key secondary endpoints, a hierarchical testing procedure will be used. Additional details will be provided in the statistical analysis plan.

The primary efficacy endpoint (the change from baseline to endpoint in the MDS-UPDRS Parts II and III combined score) will be analyzed using a Mixed Model for Repeated Measures (MMRM). The change from baseline at each postbaseline timepoint will be included in the MMRM as repeated measures. The baseline score will be included as a covariate, and treatment group, visit, the interaction between treatment group and visit, and the stratification factor (concurrent use of MAO-B inhibitor; yes/no) will be included as fixed factors. An unstructured covariance structure will be utilized. The difference between tavapadon and placebo at endpoint will be estimated based on the least square means (LSMeans) from the MMRM. A hypothetical strategy will be used to address intercurrent events (ICEs) of potential death, treatment discontinuations, missed visits/assessments, and start of prohibited concomitant medications. The data after treatment discontinuations or after the start of prohibited concomitant medications will be censored under the hypothetical strategy. The missing values are assumed to be missing at random (MAR) in the primary analysis, including missing due to COVID-19 control measures. Sensitivity analyses will be performed to assess the impact of deviation from MAR assumptions on the analysis results.

The key secondary endpoint of the change from baseline to endpoint in the MDS-UPDRS Part II score will be analyzed using an MMRM analysis similar to the analysis of the primary endpoint. Similar sensitivity analyses will also be performed.

The key secondary endpoint of the percentage of responders at endpoint, defined as a score of “much improved” or “very much improved” on the Patient Global Impression of Change (PGIC), will be analyzed using the SAS® GLIMMIX procedure for binomial data with logit link. This generalized linear mixed model analysis will include response data from each postbaseline visit as repeated measures with treatment group, visit, and interaction between treatment group and visit as fixed effects. An unstructured covariance structure will be used for the repeated measures. If the model fails to converge with the unstructured covariance structure, the heterogeneous Toeplitz structure or further reduced covariance structure may be used.

Other secondary endpoints will be analyzed similarly to the primary efficacy endpoint if the data are of a continuous nature, and similarly to the key secondary endpoint of percentage of responders on the PGIC at endpoint if the data are of ordered categorical nature.

Safety Analyses: Treatment-emergent adverse events (TEAEs) will be coded according to the Medical Dictionary for Regulatory Activities (MedDRA) and summarized by treatment group, system organ class, and preferred term. Additional summaries by seriousness, severity, relationship to IMP, and dose at the time of onset will also be prepared. Other safety endpoints (as listed above) will be summarized with descriptive statistics. Abuse potential will be assessed through active monitoring of events subject to additional monitoring (ESAMs; i.e., TEAEs related to abuse potential and TEAEs related to medication handling irregularities [MHIs]).

Inclusion Criteria: Subjects are eligible to be included in the trial only if all of the following criteria apply:

### General and Administrative:

1. Male and female subjects aged 40 to 80 years, inclusive, at the time of signing the ICF
2. Sexually active men or women of childbearing potential must agree to use acceptable (at minimum) or highly effective birth control, or remain abstinent during the trial and for 4 weeks after the last dose of trial treatment.
3. Subjects who are capable of giving signed informed consent, which includes compliance with the requirements and restrictions listed in the ICF and in this protocol.
4. Subjects who are able, in the opinion of the investigator, to understand the nature of the trial and comply with protocol requirements, including the prescribed dosage regimens, scheduled visits, laboratory tests, and other trial procedures

### Parkinson's Disease Diagnosis

5. Subjects with a diagnosis of PD that is consistent with the UK Parkinson's Disease Society Brain Bank diagnostic criteria.
   Note: Subjects having more than one affected relative (Step 2 UK Parkinson's Disease Society Brain Bank exclusion criterion) but meeting all other criteria consistent with the UK criteria will be considered to have met eligibility criteria for a diagnosis of PD.
   Note: Motor asymmetry may or may not be present at the time of Screening, and is not an explicit requirement for inclusion, as long as the subject meets the UK Parkinson's Disease Society Brain Bank diagnostic criteria based on medical history or physical examination.
6. Subjects with modified Hoehn and Yahr stage 1, 1.5, or 2 in the “on” state.
7. Subjects with disease duration (from time of diagnosis) of <3 years and disease progression in the 3 years before signing the ICF.
8. Subjects with an MDS-UPDRS Part II score≥2 and Part III score≥10 at the Screening Visit and at the Baseline Visit
9. Subjects with early PD who, in the opinion of the investigator, require pharmacologic intervention for disease management

### Concomitant Parkinson's Disease Medications

10. Subjects who are treatment naïve or have a history of prior incidental treatment with dopaminergic agents (including L-Dopa and dopamine receptor agonist medications) for <3 months in total but not within 2 months of the Baseline Visit. Prior and concurrent use of MAO-B inhibitors is permitted if use was initiated >90 days before the Baseline Visit and the dosage will remain stable for the duration of the trial (i.e., no change in the MAO-B inhibitor dose is permitted during the trial)
11. Subjects who are willing and able to refrain from any PD medications that are not permitted by the protocol (including dopaminergic agents) throughout participation in the trial.

Abbreviations: ICF=informed consent form, IMP=investigational medicinal product, L-Dopa=levodopa, MAO-B=monoamine oxidase B, MDS-UPDRS=Movement Disorder Society-Unified Parkinson's Disease Rating Scale, PD=Parkinson's disease.

### Exclusion Criteria

Subjects are excluded from the trial if any of the following criteria apply:

### Parkinson's Disease Diagnosis

1. Subjects with a history or clinical features consistent with essential tremor, atypical or secondary parkinsonian syndrome (including, but not limited to, progressive supranuclear palsy, multiple system atrophy, cortico-basal degeneration, or drug-induced or poststroke parkinsonism).
2. Subjects with a history of nonresponse or insufficient response to L-Dopa or 2 or more other antiparkinsonian drugs at therapeutic dosages.
3. Subjects who have had previous surgical intervention (e.g., deep brain stimulation) for PD or for whom such a procedure is planned or anticipated during the trial period.

### Medical History

4. Subjects with an acute or chronic, clinically significant medical or psychiatric condition, cognitive impairment, or laboratory abnormality that might increase the risk associated with trial participation or administration of trial treatment or interfere with the interpretation of the trial results or that, in the judgment of the investigator, would make the subject inappropriate for entry into this trial.
   Medical conditions that are minor or well controlled may be considered acceptable if the condition does not expose the subject to an undue risk of a significant AE or interfere with the assessments of safety or efficacy during the course of the trial. Subjects with symptoms of anxiety or depression that are not debilitating and that are stable or adequately controlled with non-prohibited medication are considered acceptable. The medical monitor should be contacted in any instance where the investigator is uncertain regarding the stability of a subject's medical conditions(s) and the potential impact of the condition(s) on trial participation.
5. Subjects with a history or current diagnosis of a clinically significant impulse control disorder (Disruptive, Impulse Control, and Conduct Disorder per DSM-5)
6. Subjects with the presence of or history of brain tumor, hospitalization for severe head trauma, epilepsy (as defined by the International League Against Epilepsy), or seizures.
7. Subjects with a history of psychosis or hallucinations within the previous 12 months based on medical records or subject/caregiver feedback.
8. Subjects who answer “yes” on the C-SSRS Suicidal Ideation Item 4 or Item 5 (Active Suicidal Ideation with Some Intent to Act, Without Specific Plan, or Active Suicidal Ideation with Specific Plan and Intent) and whose most recent episode meeting the criteria for C-SSRS Item 4 or Item 5 occurred within the last 6 months, OR Subjects who answer “yes” on any of the 5 C-SSRS Suicidal Behavior Items (actual attempt, interrupted attempt, aborted attempt, preparatory acts, or behavior) and whose most recent episode meeting the criteria for any of these 5 C-SSRS Suicidal Behavior Items occurred within the last 2 years, OR Subjects who, in the opinion of the investigator, present a serious risk of suicide, OR
9. Subjects with substance abuse or dependence disorder, including alcohol, benzodiazepines, and opioids, but excluding nicotine, within the past 6 months (180 days).
10. Subjects with dementia or cognitive impairment that, in the judgement of the investigator, would exclude the subject from understanding the ICF or participating in the trial.
11. Subjects with any condition that could possibly affect drug absorption, including bowel resections, bariatric weight loss surgery, or gastrectomy (this does not include gastric banding).
12. Subjects who have a positive result for HIV antibodies, HbsAg, or HCV antibodies at screening.
Note: Subjects who were previously infected with hepatitis C but have been successfully treated (defined as a sustained virologic response or undetectable hepatitis C viral RNA levels at 12 or more weeks after the end of treatment) may be enrolled after discussion with the medical monitor.
13. Subjects with a history of malignancy other than:
   Non-metastatic basal or squamous cell carcinoma of the skin or carcinoma in situ that was surgically removed in total>1 year before signing the ICF and had not recurred
   Another type of malignancy that had been in remission for ≥5 years before signing the ICF and had not recurred
14. Subjects with a history of myocardial infarction with residual atrial, nodal, or ventricular arrhythmias that are not controlled with medical and/or surgical intervention; second- or third-degree atrioventricular block; sick sinus syndrome; severe or unstable angina; or congestive heart failure within the last 12 months. A recent (≤12 months) history of myocardial infarction with secondary arrhythmias is exclusionary regardless of the therapeutic control.
15. Subjects with a history of neuroleptic malignant syndrome.
16. Female subjects who are breastfeeding and/or who have a positive pregnancy test result prior to receiving IMP.

### Prior or Concomitant Medications

17. Subjects who are currently receiving moderate or strong CYP3A4 inducers or CYP3A4 inhibitors (except for topical administration).
18. Subjects with a positive urine drug screen for illicit drugs are excluded and may not be retested or rescreened. Subjects with a positive urine drug screen resulting from use of marijuana (any THC-containing product), prescription, or over-the-counter medications or products that, in the investigator's documented opinion, do not signal a clinical condition that would impact the safety of the subject or interpretation of the trial results may continue evaluation for the trial following consultation and approval by the medical monitor.
19. Subjects who are using prohibited medications prior to randomization or who would be likely to require the use of prohibited concomitant medications during the trial.

### Screening Assessments

20. Subjects with a MoCA score<26.
21. Subjects with a supine blood pressure≥160 mmHg (systolic) or ≥100 mmHg (diastolic) at screening. The average of two supine measurements will be used to assess eligibility.
22. Subjects with clinically significant orthostatic hypotension (e.g., syncope).
23. Subjects with a 12-lead ECG demonstrating a QTcF interval>450 msec
At screening:
   If the QTcF interval is >450 msec on the machine reading, the ECG should be repeated with 2 additional recordings. Based on the QTcF intervals that are reported by the central service, a subject will be excluded if the QTcF interval is >450 msec on 2 or more of the 3 ECG recordings, unless due to ventricular pacing.
At baseline:
   If the QTcF interval is >450 msec on the machine readings, consult the medical monitor to determine whether the subject remains eligible to be randomized while awaiting the readings from the central service.
24. Subjects with moderate or severe renal impairment (creatinine clearance as estimated by Cockcroft-Gault formula<30 mL/min or on dialysis).
25. Subjects with any of the following abnormalities in clinical laboratory tests at the Screening Visit, as assessed by the central laboratory and confirmed by a single repeat measurement, if deemed necessary:
   AST or ALT≥3×ULN.
   Total bilirubin≥1.5×ULN. Subjects with a history of Gilbert's syndrome may be eligible provided they have a value<ULN for direct bilirubin.
26. Subjects with other abnormal laboratory test results, vital sign results, or ECG findings unless, in the judgment of the investigator, the findings are not medically significant and would not impact the safety of the subjects or the interpretation of the trial results. The medical monitor should be contacted to discuss individual cases, as needed. Tests with exclusionary results should be repeated to ensure reproducibility of the abnormality before excluding a subject based on the criteria provided in the protocol. For medically significant or exclusory abnormal ECGs results, 2 additional ECG recordings should be collected, to ensure reproducibility of the abnormality, and the 3 ECG recordings read by the central service to confirm the abnormality before excluding a subject.

### Other

27. Subjects who previously participated in any tavapadon trial, including this trial, and received IMP.
28. Subjects who received treatment with any other investigational drug within 60 days before signing the ICF.
29. Any subject who, in the opinion of the sponsor, investigator, or medical monitor, should not participate in the trial.

Abbreviations: AE=adverse event, ALT=alanine aminotransferase, AST=aspartate aminotransferase, C-SSRS=Columbia-Suicide Severity Rating Scale, CYP=cytochrome P450, DSM-5=Diagnostic and Statistical Manual of Mental Disorders, 5th Edition, ECG=electrocardiogram, HbsAg=hepatitis B surface antigen, HCV=hepatitis C virus, HIV=human immunodeficiency virus, ICF=informed consent form, IMP=investigational medicinal product, L-Dopa=levodopa, MoCA=Montreal Cognitive Assessment, PD=Parkinson's disease, QTcF=QT interval as corrected for heart rate by Fridericia's formula, THC=tetrahydrocannabinol, ULN=upper limit of normal.

### 7.3 Example 3: A Phase 3, Double-Blind, Randomized, Placebo-Controlled, Parallel Group, 27-Week Trial to Evaluate the Efficacy, Safety, and Tolerability of Tavapadon as Adjunctive Therapy for Parkinson's Disease in Levodopa-Treated Adults with Motor Fluctuations (TEMPO-3 Trial)

This is a prospective, Phase 3, multicenter, multinational, randomized, double-blind, placebo-controlled, parallel-group, 27-week trial to evaluate the efficacy, safety, tolerability, and PK of tavapadon as adjunctive therapy to levodopa (L-Dopa) in male and female subjects aged 40 to 80 years who have a diagnosis of PD (consistent with the UK Parkinson's Disease Society Brain Bank diagnostic criteria); a modified Hoehn and Yahr score of 2, 2.5, or 3 in the “on” state; a minimum of 21 hours of “off” time on 2 consecutive days; and a good response to L-Dopa in the judgment of the investigator. The trial will include a Screening Period (maximum of 4 weeks), a 27-week Treatment Period, and a 4-week Safety Follow-up Period.

### Objectives and Endpoints:

The objectives and efficacy, safety, and pharmacokinetic (PK) endpoints of the trial are summarized below.
Primary objective: To assess the effect of tavapadon on the change from baseline in total daily hours of “on” time without troublesome dyskinesia in L-Dopa-treated subjects with PD who are experiencing motor fluctuations
Secondary objective: To assess the effect of tavapadon on the change from baseline in total daily hours of “on” time without troublesome dyskinesia in L-Dopa-treated subjects with PD who are experiencing motor fluctuations

### Primary Efficacy Endpoint

Change from baseline in total daily hours of “on” time without troublesome dyskinesia in L-Dopa-treated subjects with PD who are experiencing motor functional status (Hauser diary)

### Key Secondary Efficacy Endpoints

Change from baseline to endpoint in total daily “off” time based on the 2-day average of the self-completed home diary for motor function status (Hauser diary)

### Secondary Efficacy Endpoints (All Time Points)

Change from baseline in the total “on” time without troublesome dyskinesia based on the 2-day average of the self-completed home diary for motor function status (Hauser diary)
Change from baseline in the total “off” time without troublesome dyskinesia based on the 2-day average of the self-completed home diary for motor function status (Hauser diary)
Change from baseline in the MDS-UPDRS Part I score
Change from baseline in the MDS-UPDRS Part II score
Change from baseline in the MDS-UPDRS Part III score

### Other Endpoints

Change from baseline in PDQ-39 score
Change from baseline in Schwab and England ADL score
Change from baseline in the EQ-5D-5L index and VAS scores

### Safety and Tolerability

To assess the safety and tolerability of tavapadon in L-Dopa-treated subjects with PD who are experiencing motor fluctuations

### Overall Design:

This is a prospective, Phase 3, multicenter, multinational, randomized, double-blind, placebo-controlled, parallel-group, 27-week trial to evaluate the efficacy, safety, tolerability, and PK of tavapadon as adjunctive therapy to levodopa (L-Dopa) in male and female subjects aged 40 to 80 years who have a diagnosis of PD (consistent with the UK Parkinson's Disease Society Brain Bank diagnostic criteria); a modified Hoehn and Yahr score of 2, 2.5, or 3 in the “on” state; a minimum of 21 hours of “off” time on 2 consecutive days; and a good response to L-Dopa in the judgment of the investigator. The trial will include a Screening Period (maximum of 4 weeks), a 27-week Treatment Period, and a 4-week Safety Follow-up Period.

Each subject will participate in the trial for up to approximately 35 weeks, including screening (up to 4 weeks), treatment (27 weeks), and posttreatment safety follow-up (4 weeks). Subjects who complete through Week 27 of the trial may have the opportunity to enter an open-label extension trial. The dosing schedule is provided below:

TABLE 6
Dosing schedule

| | | |
|---|---|---|
| | Titration | |
| Trial Day | Step | Blinded Treatment Assignment |

Blinded Dose Titration Phase^{a}

| | | |
|---|---|---|
| Days 1-4: | Step 1 | 0.25 mg tavapadon or placebo QD |
| Days 5-8: | Step 2 | 0.5 mg tavapadon or placebo QD |
| Days 9-12: | Step 3 | 0.75 mg tavapadon or placebo QD |
| Days 13-16: | Step 4 | 1 mg tavapadon or placebo QD |
| Days 17-20: | Step 5 | 1.5 mg tavapadon or placebo QD |
| Days 21-24: | Step 6 | 2.25 mg tavapadon or placebo QD |
| Days 25-40: | Step 7 | 3 mg tavapadon or placebo QD |
| Days 41-56: | Step 8 | 5 mg tavapadon or placebo QD |

Blinded Dose Adjustment Phase^{b}

| | | |
|---|---|---|
| Days 57-72: | Step 9 | 10 mg tavapadon or placebo QD |
| Days 73-189: | Step 10 | 15 mg tavapadon or placebo QD |

Blinded Maintenance Phase^{c}

| | | |
|---|---|---|
| Days 105-189 | Step 11 | Maximum tolerated tavapadon |
| | | dose (5-15 mg) or placebo QD |

QD—once daily
^{a}No deviations in the titration schedule up through 5 mg QD (Step 8) will be allowed.
^{b}The dose will be adjusted over the range of 5 to 15 mg (Step 9 and Step 10) based on individual subject tolerability.
^{c}Subjects will receive the highest tolerated dose level that is achieved during the Dose Adjustment Phase. No adjustment in the tavapadon dose will be allowed during the Maintenance Phase.

Titration of tavapadon should be guided by absence of tolerability issues that are reported as AEs and that are of sufficient severity resulting in significant dysfunction or distress to the subject. Any questions regarding tolerability issues and titration should be directed to the medical monitor before adjustments are initiated. The dose of tavapadon will be gradually titrated to the Step 10 dose (15 mg QD) in all subjects, as shown Table 6, unless prevented by intolerance.

Subjects who are unable to achieve or tolerate the Step 10 dose (15 mg QD) may receive the Step 9 dose (10 mg QD). Subjects who cannot achieve or tolerate the Step 9 dose (10 mg QD) may receive the Step 8 dose (5 mg QD). Subjects who cannot achieve or tolerate the Step 8 dose (5 mg QD) will be discontinued from the trial. Subjects who require a dose reduction may be rechallenged with a higher dose to address symptomatic needs after at least 7 days at the lower dose during the Dose Adjustment Phase.

Subjects will receive the highest tolerated dose level that is achieved during the Dose Adjustment Phase for the duration of the subsequent 13-week Maintenance Phase. Adjustments in the dose of tavapadon will not be allowed during the Maintenance Phase. Subjects who cannot tolerate their maintenance dose will be discontinued from the trial.

The first dose of IMP will be taken at the trial site at the Baseline Visit; all other doses will be taken on an outpatient basis. Assessments will be conducted in the clinic at the end of Weeks 2, 5, 8, 11, 14, 18, 22, 26, and 27. Subjects will complete the Hauser diary on 2 consecutive days within the 7 days before each scheduled postbaseline clinic visit. Blood samples for PK will be collected after administration of the first dose on Day 1 (˜1 hour after dosing) and at the Weeks 5, 11, 14, 22, and 27 clinic visits.

Number of Subjects: A total of 368 subjects are planned to be randomized into 2 treatment groups (184 subjects per treatment group).

Key Entry Criteria: Male and female subjects aged 40 to 80 years who have a diagnosis of PD (consistent with the UK Parkinson's Disease Society Brain Bank diagnostic criteria), a modified Hoehn and Yahr score of 2, 2.5, or 3 in the “on” state, a minimum of 22 hours of “off” time on 2 consecutive days, and a good response to L-Dopa in the judgment of the investigator will be enrolled.

Intervention Groups, Trial Treatments, and Duration: Subjects will be randomized in a 1:1 ratio to receive tavapadon 5 to 15 mg QD or placebo QD. The planned duration of treatment is 27 weeks, including the Dose Titration, Dose Adjustment, and Maintenance Phases. The IMPs will be taken orally.

Interim Analysis Review Committee: Given that the assumptions for the sample size were based on a preliminary study using a different dosing schedule, it is unknown if these assumptions are appropriate for this study as designed. To this end, an interim analysis of the primary efficacy endpoint will be included to assess the adequacy of the overall sample size relative to achieving the study objectives. This analysis will be conducted by an independent Interim Analysis Review Committee (IARC), which may make a recommendation to increase the overall sample size up to a maximum of 528 subjects. Full details and rules for the IARC and planned interim analysis will be provided in a separate IARC charter and in the statistical analysis plan.

### Statistical Methods

Sample Size Estimation: A sample size of 184 subjects per group (total, 368 subjects) will provide at least 90% power to detect a change in the primary outcome measure (change from baseline in “on” time without troublesome dyskinesia) of 1 hour with a standard deviation of 2.5, a 2-sided alpha level=0.049, and assuming a 27% dropout rate.

In the event of higher than anticipated early terminations due to COVID-19 or other reasons, enrollment may extend in order to maintain the planned statistical power.

Efficacy Analyses: The analysis of the primary and each secondary endpoint will include the comparison of tavapadon versus placebo. The hypothesis testing will be done in hierarchical order from the primary endpoint onward. To control for any potential inflation of Type I error that may be incurred from the interim analysis, the primary hypothesis will be tested at an a level of 0.049 (2-sided) to ensure that the overall Type I error rate is below 0.05. Subsequent testing in the secondary endpoints will be conducted at an a level of 0.05 (2-sided).

The primary endpoint (change from baseline to endpoint in “on” time without troublesome dyskinesia) will be analyzed using a Mixed Model for Repeated Measures (MMRM) on the modified intent-to-treat (mITT) population. This estimand will be based on the 2-day average of the self-completed Hauser diary from each postbaseline visit. A hypothetical strategy will be used to address the intercurrent events of discontinuation or use of prohibited medications with the data post intercurrent events treated as censored. The baseline value will be included as a covariate, and the treatment group (tavapadon or placebo), visit, and interaction between treatment group and visit will be included as fixed factors in the MMRM. The difference between tavapadon versus placebo at endpoint will be estimated based on the least square means (LSMeans) from the MMRM

The key secondary endpoint (change from baseline to endpoint in “off” time) and other continuous secondary endpoints will be analyzed in a similar manner to the primary endpoint. Categorical endpoints will be analyzed on the mITT population using the SAS® GLIMMIX procedure for binomial data with logit link. This generalized linear mixed model analysis will include response data from each postbaseline visit that occurs before discontinuation of treatment or addition of rescue medication through endpoint. The model will include the treatment group, visit, and interaction between treatment group and visit. An unstructured covariance structure will be used for the repeated measures. If the model fails to converge with the unstructured covariance structure, the heterogeneous Toeplitz structure or further reduced covariance structure may be used.

Safety Analyses: Treatment-emergent adverse events (TEAEs) will be coded according to the Medical Dictionary for Regulatory Activities (MedDRA) and summarized by treatment group, system organ class, and preferred term. Additional summaries by seriousness, severity, relationship to IMP, and dose at the time of onset will also be prepared. Other safety endpoints (as listed above) will be summarized with descriptive statistics. Abuse potential will be assessed through active monitoring of events subject to additional monitoring (ESAMs; i.e., TEAEs related to abuse potential and TEAEs related to medication handling irregularities [MHIs]).

Inclusion Criteria: Subjects are eligible to be included in the trial only if all of the following criteria apply:

### General and Administrative:

12. Male and female subjects aged 40 to 80 years, inclusive, at the time of signing the ICF
13. Sexually active men or women of childbearing potential must agree to use acceptable (at minimum) or highly effective birth control, or remain abstinent during the trial and for 4 weeks after the last dose of trial treatment.
14. Subjects who are capable of giving signed informed consent, which includes compliance with the requirements and restrictions listed in the ICF and in this protocol.
15. Subjects who are able, in the opinion of the investigator, to understand the nature of the trial and comply with protocol requirements, including the prescribed dosage regimens, scheduled visits, laboratory tests, and other trial procedures

### Parkinson's Disease Diagnosis

16. Subjects with a diagnosis of PD that is consistent with the UK Parkinson's Disease Society Brain Bank diagnostic criteria.
   Note: Subjects having more than one affected relative (Step 2 UK Parkinson's Disease Society Brain Bank exclusion criterion) but meeting all other criteria consistent with the UK criteria will be considered to have met eligibility criteria for a diagnosis of PD.
   Note: Motor asymmetry may or may not be present at the time of Screening, and is not an explicit requirement for inclusion, as long as the subject meets the UK Parkinson's Disease Society Brain Bank diagnostic criteria based on medical history or physical examination.
17. Subjects with modified Hoehn and Yahr stage 2, 2.5, or 3 in the “on” state.
18. Subjects with a good response to L-Dopa in the judgment of the investigator.
   Note: Subjects using levodopa/carbidopa intestinal gel are not eligible to participate in the trial
19. Subjects who return a completed self-reported home diary for motor function status (Hauser diary) during the screening period (after diary training and concordance testing has occurred), with recordings for 2 consecutive days (i.e., 2 consecutive 24-hour periods) showing at least 2½ hours of “off” time on each of the 2 days.

### Concomitant Parkinson's Disease Medications

20. Subjects who are on a stable dose of L-Dopa for at least 4 weeks prior to screening and are taking a minimum total daily dose of 400 mg divided in at least 4 doses per day of standard carbidopa/levodopa or divided in at least 3 doses per day of extended-release carbidopa/levodopa capsules. The carbidopa/levodopa dose and frequency must be maintained for the duration of the trial.
21. Prior and concurrent use of COMT inhibitors, MAO-B inhibitors, amantadine, istradefylline, or anticholinergic drugs is permitted if use was initiated >90 days before the Baseline Visit and the dosage will remain stable for the duration of the trial (i.e., no change in the COMT, MAO-B inhibitor, amantadine, istradefylline, or anticholinergic dose is permitted during the trial).

Abbreviations: COMT=catechol-O-methyl transferase, ICF=informed consent form, IMP=investigational medicinal product, L-Dopa=levodopa, MAO-B=monoamine oxidase inhibitor B, PD=Parkinson's disease.

### Exclusion Criteria

Subjects are excluded from the trial if any of the following criteria apply:

### Parkinson's Disease Diagnosis

1. Subjects with a history or clinical features consistent with essential tremor, atypical or secondary parkinsonian syndrome (including, but not limited to, progressive supranuclear palsy, multiple system atrophy, cortico-basal degeneration, or drug-induced or poststroke parkinsonism).
2. Subjects with a history of nonresponse or insufficient response to L-Dopa at therapeutic dosages.
3. Subjects who have had previous surgical intervention (e.g., deep brain stimulation) for PD or for whom such a procedure is planned or anticipated during the trial period.

### Medical History

4. Subjects with an acute or chronic, clinically significant medical or psychiatric condition, cognitive impairment, or laboratory abnormality that might increase the risk associated with trial participation or administration of trial treatment or interfere with the interpretation of the trial results or that, in the judgment of the investigator, would make the subject inappropriate for entry into this trial.
Medical conditions that are minor or well controlled may be considered acceptable if the condition does not expose the subject to an undue risk of a significant AE or interfere with the assessments of safety or efficacy during the course of the trial. Subjects with symptoms of anxiety or depression that are not debilitating and that are stable or adequately controlled with non-prohibited medication are considered acceptable. The medical monitor should be contacted in any instance where the investigator is uncertain regarding the stability of a subject's medical conditions(s) and the potential impact of the condition(s) on trial participation.
5. Subjects with a history or current diagnosis of a clinically significant impulse control disorder (Disruptive, Impulse Control, and Conduct Disorder per DSM-5) (American Psychiatric Association, 2013).
6. Subjects with the presence of or history of brain tumor, hospitalization for severe head trauma, epilepsy (as defined by the International League Against Epilepsy), or seizures.
7. Subjects with a history of psychosis or hallucinations within the previous 12 months.
8. Subjects who answer “yes” on the C-SSRS Suicidal Ideation Item 4 or Item 5 (Active Suicidal Ideation with Some Intent to Act, Without Specific Plan, or Active Suicidal Ideation with Specific Plan and Intent) and whose most recent episode meeting the criteria for C-SSRS Item 4 or Item 5 occurred within the last 6 months, OR Subjects who answer “yes” on any of the 5 C-SSRS Suicidal Behavior Items (actual attempt, interrupted attempt, aborted attempt, preparatory acts, or behavior) and whose most recent episode meeting the criteria for any of these 5 C-SSRS Suicidal Behavior Items occurred within the last 2 years, OR
Subjects who, in the opinion of the investigator, present a serious risk of suicide.
9. Subjects with substance abuse or dependence disorder, including alcohol, benzodiazepines, and opioids, but excluding nicotine, within the past 6 months (180 days).
10. Subjects with dementia or cognitive impairment that, in the judgement of the investigator, would exclude the subject from understanding the ICF or participating in the trial.
11. Subjects with any condition that could possibly affect drug absorption, including bowel resections, bariatric weight loss surgery, or gastrectomy (this does not include gastric banding).
12. Subjects who have a positive result for HIV antibodies, HbsAg, or HCV antibodies at screening.
Note: Subjects who were previously infected with hepatitis C but have been successfully treated (defined as a sustained virologic response or undetectable hepatitis C viral RNA levels at 12 or more weeks after the end of treatment) may be enrolled after discussion with the medical monitor.
13. Subjects with a history of malignancy other than:
   Non-metastatic basal or squamous cell carcinoma of the skin or carcinoma in situ that was surgically removed in total>1 year before signing the ICF and had not recurred
   Another type of malignancy that had been in remission for ≥5 years before signing the ICF and had not recurred
14. Subjects with a history of myocardial infarction with residual atrial, nodal, or ventricular arrhythmias that are not controlled with medical and/or surgical intervention; second- or third-degree atrioventricular block; sick sinus syndrome; severe or unstable angina; or congestive heart failure within the last 12 months. A recent (≤12 months) history of myocardial infarction with secondary arrhythmias is exclusionary regardless of the therapeutic control.
15. Subjects with a history of neuroleptic malignant syndrome.
16. Female subjects who are breastfeeding and/or who have a positive pregnancy test result prior to receiving IMP.

### Prior or Concomitant Medications

17. Subjects who are currently receiving moderate or strong CYP3A4 inducers or CYP3A4 inhibitors (except for topical administration).
18. Subjects with a positive urine drug screen for illicit drugs are excluded and may not be retested or rescreened. Subjects with a positive urine drug screen resulting from use of marijuana (any THC-containing product), prescription, or over-the-counter medications or products that, in the investigator's documented opinion, do not signal a clinical condition that would impact the safety of the subject or interpretation of the trial results may continue evaluation for the trial following consultation and approval by the medical monitor.
19. Subjects who are using prohibited medications prior to randomization or who would be likely to require the use of prohibited concomitant medications during the trial.

### Screening Assessments

20. Subjects with a MoCA score<26.
21. Subjects with a supine blood pressure≥160 mmHg (systolic) or ≥100 mmHg (diastolic) at screening. The average of two supine measurements will be used to assess eligibility.
22. Subjects with clinically significant orthostatic hypotension (e.g., syncope).
23. Subjects with a 12-lead ECG demonstrating a QTcF interval>450 msec
At screening:
   If the QTcF interval is >450 msec on the machine reading, the ECG should be repeated with 2 additional recordings. Based on the QTcF intervals that are reported by the central service, a subject will be excluded if the QTcF interval is >450 msec on 2 or more of the 3 ECG recordings, unless due to ventricular pacing.
At baseline:
   If the QTcF interval is >450 msec on the machine readings, consult the medical monitor to determine whether the subject remains eligible to be randomized while awaiting the readings from the central service.
24. Subjects with moderate or severe renal impairment (creatinine clearance as estimated by Cockcroft-Gault formula<30 mL/min or on dialysis).
25. Subjects with any of the following abnormalities in clinical laboratory tests at the Screening Visit, as assessed by the central laboratory and confirmed by a single repeat measurement, if deemed necessary:
   AST or ALT≥3×ULN.
   Total bilirubin≥1.5×ULN. Subjects with a history of Gilbert's syndrome may be eligible provided they have a value<ULN for direct bilirubin.
26. Subjects with other abnormal laboratory test results, vital sign results, or ECG findings unless, in the judgment of the investigator, the findings are not medically significant and would not impact the safety of the subjects or the interpretation of the trial results. The medical monitor should be contacted to discuss individual cases, as needed. Tests with exclusionary results should be repeated to ensure reproducibility of the abnormality before excluding a subject based on the criteria provided in the protocol. For medically significant or exclusory abnormal ECGs results, 2 additional ECG recordings should be collected, to ensure reproducibility of the abnormality, and the 3 ECG recordings read by the central service to confirm the abnormality before excluding a subject.

### Other

27. Subjects who previously participated in any tavapadon trial, including this trial, and received IMP.
28. Subjects who received treatment with any other investigational drug within 60 days before signing the ICF.
29. Any subject who, in the opinion of the sponsor, investigator, or medical monitor, should not participate in the trial.

Abbreviations: AE=adverse event, ALT=alanine aminotransferase, AST=aspartate aminotransferase, C-SSRS=Columbia-Suicide Severity Rating Scale, CYP=cytochrome P450, DSM-5=Diagnostic and Statistical Manual of Mental Disorders, 5th Edition, ECG=electrocardiogram, HbsAg=hepatitis B surface antigen, HCV=hepatitis C virus, HIV=human immunodeficiency virus, ICF=informed consent form, IMP=investigational medicinal product, L-Dopa=levodopa, MoCA=Montreal Cognitive Assessment, PD=Parkinson's disease, QTcF=QT interval as corrected for heart rate by Fridericia's formula, THC=tetrahydrocannabinol, ULN=upper limit of normal.

### Results

### Efficacy Objectives:

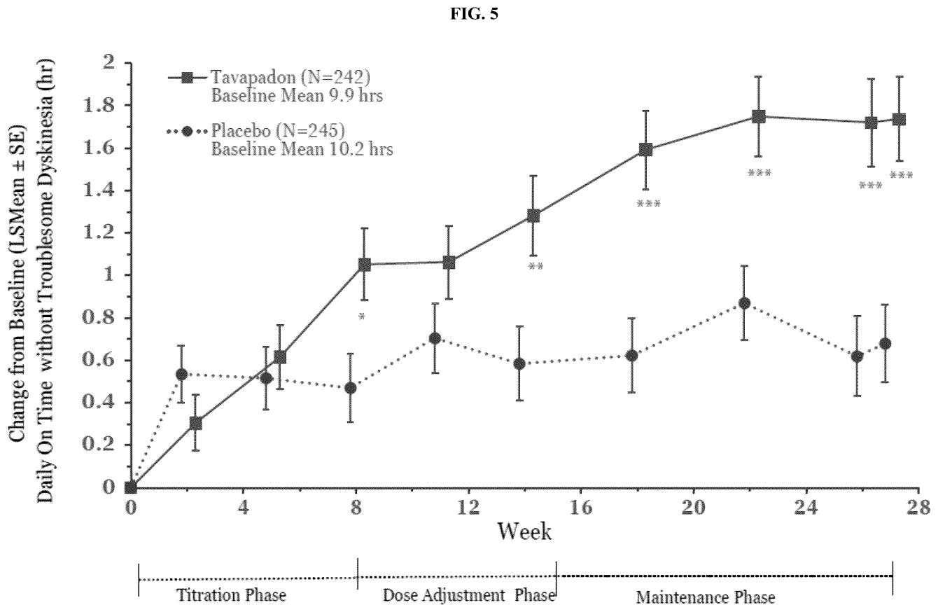

Primary: Tavapadon increased the daily “on” time without troublesome dyskinesia as adjunctive therapy to levodopa at Week 26 by 1.1 hours as compared to placebo (LS means of 1.7 hours increase from baseline in tavapadon group versus 0.6 hours increase in placebo group) with p<0.0001 (Cohen's D effect size 0.4) as seen in FIG. 5.

The increase in the daily “on” time without troublesome dyskinesia was consistent across subgroups of the trial population in prespecified subgroup analyses.

In subgroups with baseline daily off time>5 hours, the increase in on time without troublesome dyskinesis versus placebo was 1.7 hours with P<0.001.

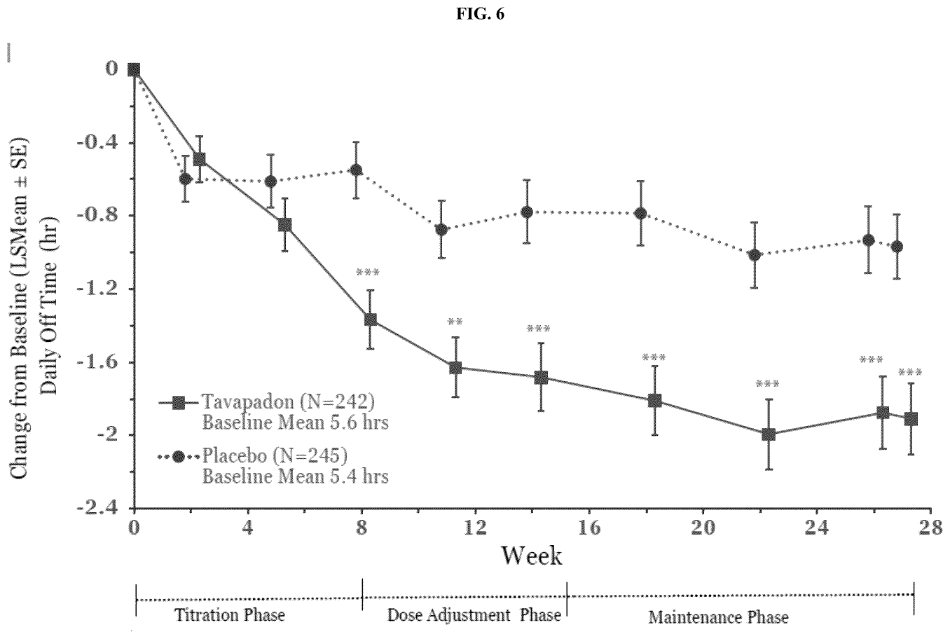

Secondary: Tavapadon reduced the daily “off” time as adjunctive therapy to levodopa at Week 26 by 0.95 hours as compared to placebo (LS means of 1.88 hours decrease from baseline in tavapadon group versus 0.93 hours decrease in placebo group) with p=0.0006 (Cohen's D effect size 0.35) as seen in FIG. 6.

The decrease in the daily “off” time was generally consistent across subgroups of the trial population in prespecified subgroup analyses.

In subgroups with baseline daily off time>5 hours, the decrease in off time versus placebo was 1.8 hours with P<0.001.

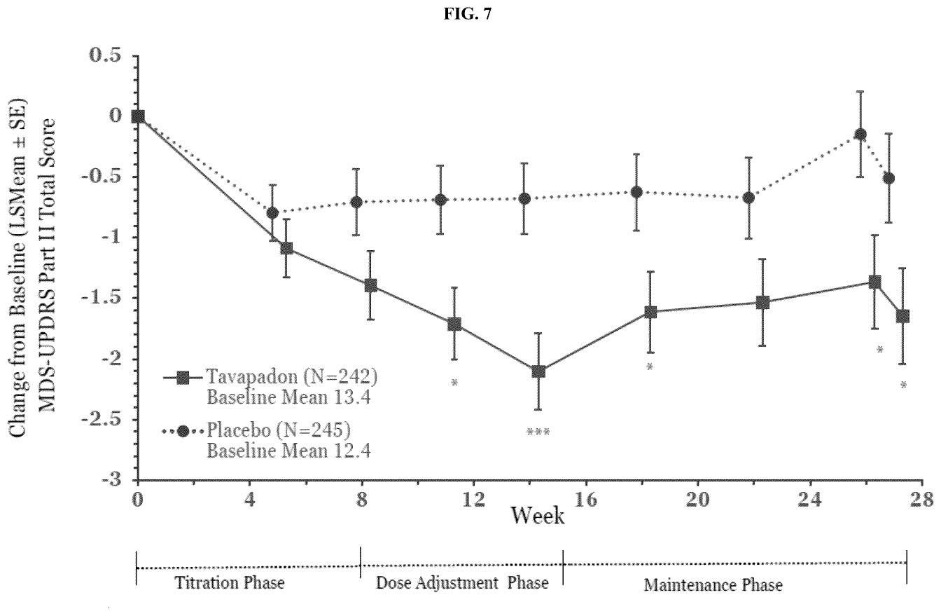

Tavapadon decreased the total score of MDS-UPDRS Part II as adjunctive therapy to levodopa at Week 26 by 1.2 points as compared to placebo (LS means of 1.4-point decrease from baseline in tavapadon group versus 0.2-point decrease in placebo group) with p=0.020 (Cohen's D effect size 0.24) as seen FIG. 7.

### Summary of Adverse Events

| | Number (%) of Subjects | | | |
|---|---|---|---|---|
| | | | Tavapadon | |
| | Placebo QD | | 5-15 mg QD | |
| | (N = 254) | | (N = 251) | |
| Subjects with Treatment | 140 | (55.1%) | 180 | (71.7%) |
| Emergent AE (TEAE) | | | | |
| Mild | 65 | (25.6%) | 80 | (31.9%) |
| Moderate | 64 | (25.2%) | 82 | (32.7%) |
| Severe | 11 | (4.3%) | 18 | (7.2%) |
| Subjects with Serious TEAE^{a} | 14 | (5.5%) | 17 | (6.8%) |
| Subjects with TEAE | 23 | (9.1%) | 43 | (17.1%) |
| Leading to Discontinuation | | | | |
| Subjects with TEAE of Special | 0 | | 3 | (1.2%)^{c} |
| Interest (AESI)^{b} without | | | | |
| Leading to Discontinuation | | | | |
| Subjects with TEAE Considered | 54 | (21.3%) | 105 | (41.8%) |
| Related to IMP | | | | |

| | | | |
|---|---|---|---|
| Death | 0 | 1 | (0.4%)^{c} |

^{a}TESAEs reported for more than one subject in tavapadon group: fall 3 (1.2%) vs placebo 0; abdominal pain 2 (0.8%) vs placebo 0.
^{b}Protocol definition of AESIs are AEs potentially related to abuse, abnormal liver function tests, or AEs leading to discontinuation
^{c}Subject 4759 (70-year old male) discontinued IMP and withdrew from the study after receiving tavapadon for 35 days. The subject died on Day 70 since randomization (after withdrawal from the study) from metastatic pancreatic carcinoma. This subject also reported an AESI not leading to discontinuation of PT Liver Function Test Abnormal which occurred in the context of the metastatic pancreatic carcinoma.

Most Common Adverse Events in Tavapadon Group are summarized below: TEAEs with ≥5% Incidence in Tavapadon Group and >2× Placebo AEs

| | Number (%) of Subjects | | |
|---|---|---|---|
| | | | Tavapadon |
| | | Placebo QD | 5-15 mg QD |
| Preferred Term | | (N = 254) | (N = 251) |

| | | | | |
|---|---|---|---|---|
| Nausea | 11 | (4.3%) | 36 | (14.3%) |
| Dyskinesia | 4 | (1.6%) | 25 | (10.0%) |
| Dizziness | 8 | (3.1%) | 19 | (7.6%) |
| Headache | 7 | (2.8%) | 17 | (6.8%) |
| Orthostatic hypotension | 3 | (1.2%) | 15 | (6.0%) |
| Hallucination, visual | 3 | (1.2%) | 14 | (5.6%) |

Majority of adverse events (AEs) reported were mild to moderate in severity. Most Common Adverse Events Occurrence by Dosing Phase are summarized below:

| | | Number (%) of Subjects | | | |
|---|---|---|---|---|---|
| | | | | Tavapadon | |
| | | Placebo QD | | 5-15 mg QD | |
| Preferred Term | Dosing Phase | (N = 254) | | (N = 251) | |
| Nausea | Titration | 10 | (3.9%) | 23 | (9.2%) |
| | Adjustment | 0 | | 12 | (5.6%) |
| | Maintenance | 1 | (0.5%) | 4 | (2.1%) |
| Dyskinesia | Titration | 4 | (1.6%) | 17 | (6.8%) |
| | Adjustment | 1 | (0.4%) | 10 | (4.7%) |
| | Maintenance | 0 | | 1 | (0.5%) |
| Dizziness | Titration | 6 | (2.4%) | 11 | (4.4%) |
| | Adjustment | 1 | (0.4%) | 9 | (4.2%) |
| | Maintenance | 1 | (0.5%) | 0 | |
| Headache | Titration | 3 | (1.2%) | 15 | (6.0%) |
| | Adjustment | 2 | (0.8%) | 2 | (0.9%) |
| | Maintenance | 2 | (0.9%) | 1 | (0.5%) |
| Orthostatic | Titration | 1 | (0.4%) | 3 | (1.2%) |
| hypotension | Adjustment | 1 | (0.4%) | 5 | (2.3%) |
| | Maintenance | 1 | (0.5%) | 7 | (3.7%) |
| Hallucination, | Titration | 1 | (0.4%) | 3 | (1.2%) |
| visual | Adjustment | 2 | (0.8%) | 6 | (2.8%) |

| | | | |
|---|---|---|---|
| Maintenance | 0 | 6 | (3.2%) |

### Safety and Tolerability

Tavapadon was generally well tolerated. The safety profile of tavapadon was consistent with previous findings and no new safety concerns were identified. Majority of adverse events (AEs) reported were mild to moderate in severity.

### 7.4 Example 4: A Phase 3, Double-Blind, Randomized, Placebo-Controlled, Parallel Group, 27-Week Trial to Evaluate the Efficacy, Safety, and Tolerability of Tavapadon as Adjunctive Therapy for Parkinson's Disease in Levodopa-Treated Adults with Motor Fluctuations (TEMPO-4 Trial)

This is a prospective, Phase 3, multicenter, multinational, open-label, 58-week trial to evaluate the safety and tolerability of tavapadon in male and female subjects aged 40 to 80 years with a diagnosis of PD.

### Objectives and Endpoints:

### Objectives:

The objectives and efficacy, safety, and pharmacokinetic (PK) endpoints of the trial are summarized below.
Safety: To evaluate the safety and tolerability of long-term administration of tavapadon in subjects with PD

### Efficacy:

To evaluate the effects of tavapadon on PD symptoms during long-term treatment

### Other:

To evaluate the effects of tavapadon on L-Dopa usage
To evaluate speech and facial expression characteristics of subjects with PD

### Pharmacokinetic:

To evaluate the PK of tavapadon in this population

### Safety Endpoints:

Nature, frequency, and temporality of TEAEs (nonserious and serious), including abuse-related AEs and AEs related to MHIs
Frequency of discontinuations

### QUIP-RS

ESS
C-SSRS
SMWQ
Clinical laboratory evaluations
Vital signs
Physical and neurological examinations
ECGs

### Efficacy Endpoints

Change from baseline in the MDS-UPDRS Parts I, II, and III scores
Change from baseline in the Hauser diary (only in subjects who require daily L-Dopa or equivalent therapy for symptom control at the time of enrollment)
Change from baseline in the EQ-5D-5L index and VAS scores

### Other Endpoints

Change from baseline in L-Dopa usage
Exploratory analyses of audio and video data collected from subject-administered remote digital testing of speech and facial expressions through an interactive application platform on the subject's personal mobile device may be conducted

### Overall Design:

This is a prospective, Phase 3, multicenter, multinational, open-label, 58-week trial to evaluate the safety and tolerability of tavapadon in male and female subjects aged 40 to 80 years with a diagnosis of PD. The trial will enroll eligible subjects who complete the Phase 3, double-blind, placebo-controlled trials of tavapadon in early or advanced PD and enter this trial within 72 hours after the last trial visit in the double-blind trial (rollover subjects) and de novo subjects with PD. The trial will include a baseline assessment, a 58-week Treatment Period (including the Dose Titration, Dose Adjustment, and Maintenance Phases), a 10-day Safety/Withdrawal Assessment Period to assess potential withdrawal symptoms after discontinuation of tavapadon, and a 20-day Safety Follow-up Period. A Screening Period of up to 4 weeks will be included for de novo subjects.

Rollover subjects will participate in the trial for up to approximately 62 weeks, including open-label treatment (58 weeks), safety/withdrawal assessment (10 days), and safety follow-up (20 days). De novo subjects will participate in the trial for up to approximately 66 weeks, including screening (up to 4 weeks), open-label treatment (58 weeks), safety/withdrawal assessment (10 days), and safety follow-up (20 days).

Each subject will participate in the trial for up to approximately 35 weeks, including screening (up to 4 weeks), treatment (27 weeks), and posttreatment safety follow-up (4 weeks). Subjects who complete through Week 27 of the trial may have the opportunity to enter an open-label extension trial. The dosing schedule is provided below:

### Dosing Schedule

| | |
|---|---|
| Trial Day | Tavapadon Dose |

Dose Titration Phase (Blinded)
No deviations in the titration schedule up through 5 mg
QD will be allowed. Subjects who are unable to tolerate
IMP up through Day 56 must discontinued from the trial

| | |
|---|---|
| Days 1-4 | 0.25 mg tavapadon QD |
| Days 5-8 | 0.5 mg tavapadon QD |
| Days 9-12 | 0.75 mg tavapadon QD |
| Days 13-16 | 1 mg tavapadon QD |
| Days 17-20 | 1.5 mg tavapadon QD |
| Days 21-24 | 2.25 mg tavapadon QD |
| Days 25-40 | 3 mg tavapadon QD |
| Days 41-56 | 5 mg tavapadon QD |

Dose Adjustment Phase (Blinded)

| | |
|---|---|
| Days 57-72 | 10 mg tavapadon QD |
| | Subjects who are unable to increase dose from 5 mg |
| | to |
| | 10 mg on Day 57 due to tolerability issues will begin |
| | open- label maintenance dosing at their maximum |
| | tolerated dose of 5 mg QD at that time. |
| | Subjects who achieve 10 mg dosing on Day 57 but |
| | who require a dose reduction due to tolerability |
| | issues between Days 58-72 will also begin open- |
| | label maintenance dosing at their maximum |
| | tolerated dose of 5 mg QD at the time of the dose |
| | reduction. |

Maintenance Phase (Open-label)
Transition to open-label maintenance dosing can occur as early as
Day 57 (5 mg QD) or as late as Day 73 (10 mg QD or 15 mg QD),
depending on the timing of determination of the maximum tolerated
dose. Adjustments in the dose of tavapadon (from 5-15 mg QD) will
be allowed during the Maintenance Phase based on individual
subject tolerability and optimized treatment response.

| | |
|---|---|
| Day 73- | 15 mg QD |
| Week 58 | Subjects unable to increase dose from 10 mg to 15 |
| | mg on Day 73 due to tolerability issues will begin |
| | open-label maintenance dosing at their maximum |
| | tolerated dose of 10 mg QD at that time |

Abbreviations:
IMP = investigational medicinal product,
QD = once daily

Abbreviations: IMP=investigational medicinal product, QD=once daily

To ensure that the treatment assignments in the Phase 3 double-blind trials remain blinded for the subjects who rollover into the open-label trial, all subjects (rollover and de novo) will be dispensed blinded tavapadon tablets during the Dose Titration and Dose Adjustment Phases. All subjects will take 3 tablets QD (either all tavapadon tablets or a combination of tavapadon and placebo tablets) during the Dose Titration and Dose Adjustment Phases until they reach the Maintenance Phase on Day 73, achieving dosing with 15 mg QD.

Subjects whose maximum tolerated dose is determined to be either 5 mg or 10 mg QD during the Dose Adjustment Phase (prior to Day 73) will begin open-label maintenance dosing at the time their maximum tolerated dose is determined.

### Blinded Dose Titration Phase (Days 1-56)

The dose of tavapadon will be gradually titrated to 5 mg QD for all subjects during the blinded Dose Titration Phase through Day 56. No deviations in the titration schedule will be allowed. Subjects who are unable to tolerate their blinded dose at any time during the Dose Titration Phase will be discontinued from the trial.

### Blinded Dose Adjustment Phase (Days 57-72)

The dose of tavapadon will be increased to 10 mg QD at the start of the blinded Dose Adjustment Phase on Day 57, and subjects will continue to receive 10 mg QD through Day 72. Subjects who are determined by the investigator to be unable to increase their dose on the final day of the blinded Dose Titration Phase (Day 56) due to tolerability issues will be considered to have achieved their maximum tolerated dose (5 mg) and will initiate open-label maintenance dosing of 5 mg QD at Day 57.

Subjects who achieve 10 mg QD dosing (blinded) on Day 57 but who require a dose reduction to 5 mg QD due to tolerability issues between Days 58 and 72 will be considered to have achieved their maximum tolerated dose (5 mg QD) and will initiate open-label maintenance dosing of 5 mg QD at the time of the dose reduction

### Open-Label Maintenance Phase (Day 73-Week 58)

The dose of tavapadon will be increased from 10 mg to 15 mg QD at the start of the open-label Maintenance Phase. Subjects who are determined by the investigator to be unable to increase their dose on Day 72 of the blinded Dose Adjustment Phase will be considered to have achieved their maximum tolerated dose (10 mg) and will initiate open-label maintenance dosing of 10 mg QD at Day 73.

Subjects whose maximum tolerated dose is determined to be 5 mg QD during the blinded Dose Adjustment Phase will begin open-label maintenance dosing at the time they achieve their maximum tolerated dose (i.e., prior to Day 73).

Adjustments in the dose of tavapadon will be allowed during the Maintenance Phase based on individual subject tolerability and optimized treatment response. Dose adjustments will be made following the same criteria as noted above for the Dose Titration and Dose Adjustment Phases. Subjects who cannot tolerate a dose of 5 mg QD during the Maintenance Phase will be discontinued from the trial.

Subjects who require a dose reduction may be rechallenged with a higher dose to address symptomatic needs after at least 7 days at the lower dose during the Maintenance Phase

Number of Subjects: Up to approximately 1200 subjects are expected to be enrolled to provide at least 300 evaluable subjects who complete 26 weeks of treatment at doses of 5 to 15 mg QD and 100 evaluable subjects who complete 52 weeks of treatment at doses of 5 to 15 mg QD.

### Key Entry Criteria:

The trial will enroll subjects who have completed one of the double-blind trials in PD (Trials CVL-751-PD-001, -002, or -003) and who, in the judgment of the investigator, demonstrated adequate compliance with the investigational medicinal product (IMP) and protocol requirements in the double-blind trial. In addition, the trial will enroll de novo subjects of either sex aged 40 to 80 years who have a diagnosis of PD (consistent with the UK Parkinson's Disease Society Brain Bank diagnostic criteria) and a modified Hoehn and Yahr stage of 1, 1.5, 2, 2.5, or 3. De novo subjects must be receiving some form of levodopa/carbidopa (or levodopa/benserazide) at the Screening Visit and must continue to use levodopa/carbidopa (or levodopa/benserazide) for the duration of the trial. Prior and/or concomitant use of levodopa (L-Dopa), catechol-O-methyl transferase (COMT) inhibitors, monoamine oxidase B (MAO-B) inhibitors, amantadine, istradefylline, and anticholinergic drugs will be permitted in all subject groups.

Intervention Groups, Trial Treatments, and Duration: All subjects will receive open-label tavapadon, 5 to 15 mg QD, administered orally during a 58-week Treatment Period.

### Statistical Methods

Sample Size Estimation: No sample size estimation is required. The trial will enroll eligible subjects who complete one of the Phase 3, double-blind, placebo-controlled trials (CVL-751-PD-001, -002, or -003), as well as de novo subjects.

Up to approximately 1200 subjects are expected to be enrolled to provide at least 300 evaluable subjects who complete 26 weeks of treatment at doses of 5 to 15 mg QD (with at least 50% at 15 mg QD) and 100 evaluable subjects who complete 52 weeks of treatment at doses of 5 to 15 mg QD (with at least 50% at 15 mg QD). In the event of higher than anticipated early terminations due to COVID-19 or other reasons, Cerevel may extend enrollment in order to achieve the target number of safety exposure at 26 and 52 weeks noted above.

### Efficacy Analyses:

Descriptive statistics will be used to display the observed and change-from-baseline values for each efficacy endpoint for the FAS population. Baseline will be defined as the last available measurement before the first dose of IMP in the open-label trial. Additional analyses may be conducted for the subjects who rollover from the double-blind trials to evaluate changes from the baseline of the double-blind trial. These analyses will be detailed in the statistical analysis plan.

### Safety Analyses:

All safety analyses will be conducted on the Full Analysis Set (FAS), defined as all subjects who receive at least 1 dose of IMP. Treatment-emergent adverse events (TEAEs) will be coded according to the Medical Dictionary for Regulatory Activities (MedDRA) and summarized by system organ class and preferred term. Additional summaries by seriousness, severity, relationship to the IMP, and dose at the time of onset will also be prepared. Descriptive statistics will be used to display the observed and change-from-baseline values for continuous safety endpoints, and counts and percentages will be used to display categorical data. Additional analyses may be conducted for the subjects who rollover from the double-blind trials to evaluate changes from the baseline of the double-blind trial. These analyses will be detailed in the statistical analysis plan.

### Inclusion Criteria:

### Inclusion Criteria—Rollover Subjects

Rollover subjects (those who enter this trial within 72 hours after completing the last trial visit in the double-blind trial) are eligible to be included in the trial only if all of the following criteria apply:

1. Subjects who complete the 27-week double-blind Treatment Period of Trial CVL-751-PD-001 or Trial CVL-751-PD-003 or the 27-week double-blind Treatment Period and 10-day Safety/Withdrawal Assessment Period of Trial CVL-751-PD-002 and enter this trial within 72 hours after completing the last trial visit in the double-blind trial. Rollover subjects from Trial CVL-751-PD-003 must continue to use levodopa/carbidopa (or levodopa/benserazide) for the duration of the trial.

2. Sexually active men or women of childbearing potential must agree to use acceptable (at minimum) or highly effective birth control, as defined in Section 10.4 (Appendix 4), or remain abstinent during the trial and for 4 weeks after the last dose of trial treatment.

3. Subjects who are capable of giving signed informed consent, which includes compliance with the requirements and restrictions listed in the ICF (informed consent form), and in this protocol.

4. Subjects who are able, in the opinion of the investigator, to understand the nature of the trial and comply with protocol requirements, including the prescribed dosage regimens, scheduled visits, laboratory tests, and other trial procedures.

5. Subjects who, in the judgement of the investigator, demonstrated adequate compliance with the IMP (investigational medicinal product) and protocol requirements in the double-blind trial.

6. Subjects who are willing and able to refrain from any PD medications that are not permitted by the protocol (including dopaminergic agents) throughout participation in the trial.

### De Novo Subjects

De Novo are eligible to be included in the trial only if all of the following criteria apply:

### General and Administrative:

1. Male and female subjects aged 40 to 80 years, inclusive, at the time of signing the ICF
2. Sexually active men or women of childbearing potential must agree to use acceptable (at minimum) or highly effective birth control, or remain abstinent during the trial and for 4 weeks after the last dose of trial treatment.
3. Subjects who are capable of giving signed informed consent, which includes compliance with the requirements and restrictions listed in the ICF and in this protocol.
4. Subjects who are able, in the opinion of the investigator, to understand the nature of the trial and comply with protocol requirements, including the prescribed dosage regimens, scheduled visits, laboratory tests, and other trial procedures.

### Parkinson's Disease Diagnosis

5. Subjects with a diagnosis of PD that is consistent with the UK Parkinson's Disease Society Brain Bank diagnostic criteria.
   Note: Subjects having more than one affected relative (Step 2 UK Parkinson's Disease Society Brain Bank exclusion criterion) but meeting all other criteria consistent with the UK criteria will be considered to have met eligibility criteria for a diagnosis of PD.
   Note: Motor asymmetry may or may not be present at the time of Screening, and is not an explicit requirement for inclusion, as long as the subject meets the UK Parkinson's Disease Society Brain Bank diagnostic criteria based on medical history or physical examination.
6. Subjects with modified Hoehn and Yahr stage 1, 1.5, 2, 2.5, or 3.

### Concomitant Parkinson's Disease Medications

7. Subjects must be receiving some form of levodopa/carbidopa at the Screening Visit and must continue to use levodopa/carbidopa for the duration of the trial. Standard or extended-release levodopa/benserazide is allowed as an alternative to levodopa/carbidopa, where available.
8. Prior and concurrent use of COMT inhibitors, MAO-B inhibitors, amantadine, istradefylline, or anticholinergic drugs is permitted.
9. Subjects who are willing and able to refrain from any PD medications that are not permitted by the protocol (including dopaminergic agents) throughout participation in the trial.

### Exclusion Criteria

### Exclusion Criteria—Rollover Subjects

Rollover subjects are excluded from the trial if any of the following criteria apply:

1. Subjects who do not enroll in this open-label trial within 72 hours after completing the last trial visit in the double-blind trial.

2. Subjects who, in the judgement of the investigator, experienced poor tolerability to the IMP during the double-blind trial or whose safety assessments resulted in new concerns that would suggest that the subject may not be appropriate for a 58-week trial of tavapadon.

3. Subjects who, in the judgment of the investigator, were noncompliant with trial procedures or with IMP administration during the double-blind trial.

4. Subjects who answer “yes” on the C-SSRS Suicidal Ideation Item 4 or Item 5 (Active Suicidal Ideation with Some Intent to Act, Without Specific Plan, or Active Suicidal Ideation with Specific Plan and Intent) and whose most recent episode meeting the criteria for C-SSRS Item 4 or Item 5 occurred within the last 6 months, OR

Subjects who answer “yes” on any of the 5 C-SSRS Suicidal Behavior Items (actual attempt, interrupted attempt, aborted attempt, preparatory acts, or behavior) and whose most recent episode meeting the criteria for any of these 5 C-SSRS Suicidal Behavior Items occurred within the last 2 years, OR
Subjects who, in the opinion of the investigator, present a serious risk of suicide.

5. Subjects who, in the judgment of the investigator, had a clinically significant medical, surgical, psychiatric, or laboratory abnormality after completing the last trial visit in the double-blind trial that would compromise participation in this trial.

6. Subjects who had previously been enrolled in this open-label trial and had subsequently withdrawn.

### Exclusion Criteria—De Novo Subjects

De novo subjects are excluded from the trial if any of the following criteria apply:

### Parkinson's Disease Diagnosis

1. Subjects with a history or clinical features consistent with essential tremor, atypical or secondary parkinsonian syndrome (including, but not limited to, progressive supranuclear palsy, multiple system atrophy, cortico-basal degeneration, or drug-induced or poststroke parkinsonism).
2. Subjects with a history of nonresponse or insufficient response to L-Dopa at therapeutic dosages based on medical records or subject/caregiver feedback.
3. Subjects who have had previous surgical intervention (e.g., deep brain stimulation) for PD or for whom such a procedure is planned or anticipated during the trial period

### Medical History

4. Subjects with an acute or chronic, clinically significant medical or psychiatric condition, cognitive impairment, or laboratory abnormality that might increase the risk associated with trial participation or administration of trial treatment or interfere with the interpretation of the trial results or that, in the judgment of the investigator, would make the subject inappropriate for entry into this trial.
Medical conditions that are minor or well controlled may be considered acceptable if the condition does not expose the subject to an undue risk of a significant AE or interfere with the assessments of safety or efficacy during the course of the trial. Subjects with symptoms of anxiety or depression that are not debilitating and that are stable or adequately controlled with non-prohibited medication are considered acceptable. The medical monitor should be contacted in any instance where the investigator is uncertain regarding the stability of a subject's medical conditions(s) and the potential impact of the condition(s) on trial participation.
5. Subjects with a history or current diagnosis of a clinically significant impulse control disorder (Disruptive, Impulse Control, and Conduct Disorder per DSM-5).
6. Subjects with the presence of or history of brain tumor, hospitalization for severe head trauma, epilepsy (as defined by the International League Against Epilepsy), or seizures.
7. Subjects with a history of psychosis or hallucinations within the previous 12 months based on medical records or subject/caregiver feedback.
8. Subjects who answer “yes” on the C-SSRS Suicidal Ideation Item 4 or Item 5 (Active Suicidal Ideation with Some Intent to Act, Without Specific Plan, or Active Suicidal Ideation with Specific Plan and Intent) and whose most recent episode meeting the criteria for C-SSRS Item 4 or Item 5 occurred within the last 6 months, OR
   Subjects who answer “yes” on any of the 5 C-SSRS Suicidal Behavior Items (actual attempt, interrupted attempt, aborted attempt, preparatory acts, or behavior) and whose most recent episode meeting the criteria for any of these 5 C-SSRS Suicidal Behavior Items occurred within the last 2 years, OR
   Subjects who, in the opinion of the investigator, present a serious risk of suicide.
9. Subjects with substance abuse or dependence disorder, including alcohol, benzodiazepines, and opioids, but excluding nicotine, within the past 6 months (180 days).
10. Subjects with dementia or cognitive impairment that, in the judgement of the investigator, would exclude the subject from understanding the ICF or participating in the trial.
11. Subjects with any condition that could possibly affect drug absorption, including bowel resections, bariatric weight loss surgery, or gastrectomy (this does not include gastric banding).
12. Subjects who have a positive result for HIV antibodies, HbsAg, or HCV antibodies at screening.
Note: Subjects who were previously infected with hepatitis C but have been successfully treated (defined as a sustained virologic response or undetectable hepatitis C viral RNA levels at 12 or more weeks after the end of treatment) may be enrolled after discussion with the medical monitor.
13. Subjects with a history of malignancy other than:
   Non-metastatic basal or squamous cell carcinoma of the skin or carcinoma in situ that was surgically removed in total>1 year before signing the ICF and had not recurred
   Another type of malignancy that had been in remission for ≥5 years before signing the ICF and had not recurred
14. Subjects with a history of myocardial infarction with residual atrial, nodal, or ventricular arrhythmias that are not controlled with medical and/or surgical intervention; second- or third-degree atrioventricular block; sick sinus syndrome; severe or unstable angina; or congestive heart failure within the last 12 months. A recent (≤12 months) history of myocardial infarction with secondary arrhythmias is exclusionary regardless of the therapeutic control.
15. Subjects with a history of neuroleptic malignant syndrome.
16. Female subjects who are breastfeeding and/or who have a positive pregnancy test result prior to receiving IMP.

### Prior or Concomitant Medications

17. Subjects who are currently receiving moderate or strong CYP3A4 inducers or CYP3A4 inhibitors (except for topical administration).
18. Subjects with a positive urine drug screen for illicit drugs are excluded and may not be retested or rescreened. Subjects with a positive urine drug screen resulting from use of marijuana (any THC-containing product), prescription, or over-the-counter medications or products that, in the investigator's documented opinion, do not signal a clinical condition that would impact the safety of the subject or interpretation of the trial results may continue evaluation for the trial following consultation and approval by the medical monitor.
19. Subjects who are using prohibited medications prior to randomization or who would be likely to require the use of prohibited concomitant medications during the trial.

### Screening Assessments

20. Subjects with a MoCA score<26.
21. Subjects with a supine blood pressure≥160 mmHg (systolic) or ≥100 mmHg (diastolic) at screening. The average of two supine measurements will be used to assess eligibility.
22. Subjects with clinically significant orthostatic hypotension (e.g., syncope).
23. Subjects with a 12-lead ECG demonstrating a QTcF interval>450 msec
At screening:
If the QTcF interval is >450 msec on the machine reading, the ECG should be repeated with 2 additional recordings. Based on the QTcF intervals that are reported by the central service, a subject will be excluded if the QTcF interval is >450 msec on 2 or more of the 3 ECG recordings, unless due to ventricular pacing.
At baseline:
If the QTcF interval is >450 msec on the machine reading, the ECG should be repeated with 2 additional recording and the medical monitor consulted to determine whether the subject remains eligible for the trial while awaiting the readings from the central service.
24. Subjects with moderate or severe renal impairment (creatinine clearance as estimated by Cockcroft-Gault formula<30 mL/min or on dialysis).
25. Subjects with any of the following abnormalities in clinical laboratory tests at the Screening Visit, as assessed by the central laboratory and confirmed by a single repeat measurement, if deemed necessary:
   AST or ALT≥3×ULN.
   Total bilirubin≥1.5×ULN. Subjects with a history of Gilbert's syndrome may be eligible provided they have a value<ULN for direct bilirubin.
26. Subjects with other abnormal laboratory test results, vital sign results, or ECG findings unless, in the judgment of the investigator, the findings are not medically significant and would not impact the safety of the subjects or the interpretation of the trial results. The medical monitor should be contacted to discuss individual cases, as needed. Tests with exclusionary results should be repeated to ensure reproducibility of the abnormality before excluding a subject based on the criteria provided in the protocol. For medically significant or exclusory abnormal ECGs results, 2 additional ECG recordings should be collected, to ensure reproducibility of the abnormality, and the 3 ECG recordings read by the central service to confirm the abnormality before excluding a subject.

### Other

27. Subjects who previously participated in any tavapadon trial, including this trial, and received IMP.
28. Subjects who received treatment with any other investigational drug within 60 days before signing the ICF.
29. Any subject who, in the opinion of the sponsor, investigator, or medical monitor, should not participate in the trial.

Abbreviations: AE=adverse event, ALT=alanine aminotransferase, AST=aspartate aminotransferase, C-SSRS=Columbia-Suicide Severity Rating Scale, CYP=cytochrome P450, DSM-5=Diagnostic and Statistical Manual of Mental Disorders, 5th Edition, ECG=electrocardiogram, HbsAg=hepatitis B surface antigen, HCV=hepatitis C virus, HIV=human immunodeficiency virus, ICF=informed consent form, IMP=investigational medicinal product, L-Dopa=levodopa, MoCA=Montreal Cognitive Assessment, PD=Parkinson's disease, QTcF=QT interval as corrected for heart rate by Fridericia's formula, THC=tetrahydrocannabinol, ULN=upper limit of normal.

### Results

This study was initiated in February 2021 and is still ongoing at the time of interim data cut of Oct. 25, 2024. A total of 992 subjects enrolled and 991 were treated with tavapadon.

Long-term treatment with tavapadon was generally safe. Most TEAEs were non-serious and mild/moderate in severity. The safety results were consistent with the safety profile observed in previous studies with no new safety concerns.

Higher rates of AEs and discontinuations seen during titration in the previously unexposed replicating the findings of the previous double-blind studies. The most common AEs in the previously unexposed were nausea, headache and dizziness consistent with previous findings in double-blind trials. The rate were lower in the previously exposed rollover cohorts.

Somnolence continues to occur less than the other oral DAs, reinforcing the benefit of D1/D5 selectivity. ICD incidence (1.4%) remains low at the end of study. Dyskinesias remain limited; as expected more in advanced PD (4.9%) compared to early PD (1.2%)

Higher rates of certain AEs in maintenance are likely driven by advanced disease stage. Orthostatic hypotension, hallucinations, and falls all generally occurred more frequently in advanced PD patients. The vast majority of falls (86/91) were not considered related to study treatment.

The efficacy of tavapadon in both early and advanced PD populations was maintained during long-term treatment with tavapadon. There were no signals or trends suggesting loss of efficacy with long-term treatment. A slight upwards trend (worsening) in the MDS-UPDRS assessments in the adjunctive population is likely due to advanced disease state.

Rollover subjects who received tavapadon during the double-blind trials [CVL-751-PD-001 (TemPo-1) and CVL-751-PD-003 (TemPo-3)] initiated treatment in the open-label trial at the dose that they were receiving at the Week 27 visit of the double-blind trial. For the rollover subjects who received placebo in the double blind trials, all rollover subjects from TemPo-2 (the treatment was discontinued after completing 27-week treatment by design to assess potential withdrawal symptoms) and de novo (on carbidopa/levodopa) subjects, the dose of tavapadon was gradually titrated and adjusted during the 10 week blinded Dose Titration and Dose Adjustment Phases until they reached the Maintenance Phase on Day 73. The blind during Dose Titration and Dose Adjustment Phases was to safeguard the blind of the rollover subjects from the double-blind studies. The treatment for the maintenance phase was open-label for all enrolled subjects.

The sample size was not based on statistical hypothesis testing in this uncontrolled study. The study planned to enroll approximately 800 subjects to provide at least 300 evaluable subjects who complete 26 weeks of treatment at doses of 5 to 15 mg QD (with at least 50% at 15 mg QD) and 100 evaluable subjects who complete 52 weeks of treatment at doses of 5 to 15 mg QD (with at least 50% at 15 mg QD).

### Demographic, Baseline Characteristics, and Subject Disposition

Subject disposition is presented below. Overall rate of discontinuation (29.3%) consistent with expectation for long-term trials. The discontinuation rate was higher in the previously unexposed population (37.0%) than the rollover population (20.7%). Main reasons for discontinuation treatment period were adverse event and withdrawal by subject. Discontinuation due to lack of efficacy was rare (0.2%).

| | | | Rollover | | | |
|---|---|---|---|---|---|---|
| | | | Population | | | |
| | Previously | | Previously | | | |
| | Unexposed | | Treated with | | | |
| | Population | | Tavapadon | | Overall | |
| | (N = 524) | | (N = 468) | | (N = 992) | |
| Disposition | n (%) | | n (%) | | n (%) | |
| Subjects completed | 242 | (46.2%) | 265 | (56.6%) | 507 | (51.1%) |
| study | | | | | | |
| Subjects ongoing as | 88 | (16.8%) | 106 | (22.6%) | 194 | (19.6%) |
| of Oct. 25, 2024 | | | | | | |
| Subjects | 194 | (37.0%) | 97 | (20.7%) | 291 | (29.3%) |
| discontinued from | | | | | | |
| study | | | | | | |
| Adverse Event | 100 | (19.1%) | 40 | (8.5%) | 140 | (14.1%) |
| Withdrawal by | 47 | (9.0%) | 29 | (6.2%) | 76 | (7.7%) |
| Subject | | | | | | |
| Site Terminated by | 15 | (2.9%) | 7 | (1.5%) | 22 | (2.2%) |
| Sponsora | | | | | | |
| Lost to Follow-up | 10 | (1.9%) | 3 | (0.6%) | 13 | (1.3%) |
| Other | 9 | (1.7%) | 2 | (0.4%) | 11 | (1.1%) |
| Use of Prohibited | 7 | (1.3%) | 3 | (0.6%) | 10 | (1.0%) |
| Concomitant | | | | | | |
| Medications | | | | | | |
| Physician Decision | 3 | (0.6%) | 4 | (0.9%) | 7 | (0.7%) |
| Death^{b} | 2 | (0.4%) | 1 | (0.2%) | 3 | (0.3%) |
| Non-Compliance | 0 | | 3 | (0.6%) | 3 | (0.3%) |
| with Study Schedule | | | | | | |
| Non-Compliance | 1 | (0.2%) | 2 | (0.4%) | 3 | (0.3%) |
| with Study | | | | | | |
| Drug | | | | | | |
| Lack of Efficacy | 0 | (0.0%) | 2 | (0.4%) | 2 | (0.2%) |
| Failure to Meet | 0 | (0.0%) | 1 | (0.2%) | 1 | (0.1%) |
| Continuation | | | | | | |
| Criteria | | | | | | |

^{a} Sites in Ukraine.
^{b}Cause of death: 6029 (53/M) Atherosclerotic coronary artery disease; 0619 (64/M) Cardiac arrest; 0261 (71/M) Pulmonary embolism.

### Safety

Long-term treatment with Tavapadon was generally safe. The safety profile of Tavapadon was consistent with findings from prior studies with no new safety concerns.

The overall incidence of TEAEs in TemPo-4 was 76.0%; however, most of the AEs reported were non-serious (89.2%) and mild/moderate in severity (87.3%).

The overall incidence of SAEs was 8.2%; The incidence was lower in previously exposed population (6.1%) versus previously unexposed population (10.1%). Overall, the only SAE reported by more than 0.5% of the overall population is Fall (11 subjects out of 991: 1.1%), all of which were considered unrelated to study treatment by the investigators.

There were 5 (0.5%) deaths reported. Two of the deaths occurred after the subjects had already discontinued study treatment. All deaths were considered not related.

The overall rates of TEAEs leading to treatment discontinuation was 14.4%. The incidence was highest during the first 12 weeks of treatment (10.2%), particularly during titration phase (11.2%). The incidence decreased to 2.4% during Week 13-24 and decreased steadily further during subsequent exposure. The incidence was 8.8% in previously exposed population and 19.5% in previously unexposed population.

The most common TEAEs were nausea (11.0%), dizziness (10.8%) and headache (9.7%).

CMQ search was conducted in key safety topics of interest. Based on the CMQ search results:
a. Orthostatic hypotension showed an overall incidence of 3.7%. The incidence rate was similar in the previously exposed population (3.8%) and previously unexposed population (3.6%).
b. Hallucination showed an overall incidence of 6.3%. The incidence rate was 4.1% in the previously exposed population and 8.2% in the previously unexposed population.
c. Somnolence showed an overall incidence of 5.0%. The incidence was comparable between previously exposed population (5.1%) and previously unexposed population (5.0%).
d. Fall showed an overall incidence of 9.2%. The vast majority of the reported cases, 86 out of 91 were considered not related.
e. Impulse Control Disorders (ICDs) showed an overall incidence of 1.4%.
f. All CNS active drugs are required to have an assessment for abuse potential. The abuse potential search strategy yielded an overall incidence of 1.5%, with only 3 PTs represented (Brain fog, Feeling abnormal, Thinking abnormal). Further review of these events did not suggest a clinical picture of abuse potential.

| | | | Rollover | | | |
|---|---|---|---|---|---|---|
| | | | Population | | | |
| | Previously | | Previously | | | |
| | Unexposed | | Treated with | | | |
| | Population | | Tavapadon | | Overall | |
| | (N = 523) | | (N = 468) | | (N = 991) | |
| | n (%) | | n (%) | | n (%) | |
| Treatment-emergent | 421 | (80.5%) | 332 | (70.9%) | 753 | (76.0%) |
| adverse event | | | | | | |
| (TEAE) | | | | | | |
| Mild | 149 | (28.5%) | 147 | (31.4%) | 296 | (29.9%) |
| Moderate | 207 | (39.6%) | 154 | (32.9%) | 361 | (36.4%) |
| Severe | 65 | (12.4%) | 31 | (6.6%) | 96 | (9.7%) |
| TEAE Related to | 288 | (55.1%) | 165 | (35.3%) | 453 | (45.7%) |
| IMP | | | | | | |
| Treatment-emergent | 53 | (10.1%) | 28 | (6.0%) | 81 | (8.2%) |
| serious adverse event | | | | | | |
| (TESAE) | | | | | | |
| TEAE Leading to | 102 | (19.5%) | 41 | (8.8%) | 143 | (14.4%) |
| Discontinuation of | | | | | | |
| Treatment | | | | | | |
| Death* | 3 | (0.6%) | 2 | (0.4%) | 5 | (0.5%) |

*Cause of death: 6029 (53/M) Atherosclerotic coronary artery disease; 0619 (64/M) Cardiac arrest; 0261 (71/M) Pulmonary embolism; 4442 (71/M) Chronic respiratory failure; 4839 (74/M): Pulmonary embolism

Summary of Most Frequent TEAEs with Overall Incidence Z≥5%—FAS

| | Previously | | Rollover Population | | |
|---|---|---|---|---|---|
| | Unexposed | | Previously Treated | | |
| | Population | | with Tavapadon | | Overall |
| Preferred | (N = 523) | | (N = 468) | | (N = 991) |
| Term | n (%) | | n (%) | | n (%) |
| Nausea | 87 | (16.6%) | 22 | (4.7%) | 109 (11.0%) |
| Dizziness | 72 | (13.8%) | 35 | (7.5%) | 107 (10.8%) |
| Headache | 75 | (14.3%) | 21 | (4.5%) | 96 (9.7%) |
| Fall | 57 | (10.9%) | 34 | (7.3%) | 91 (9.2%) |
| COVID-19 | 39 | (7.5%) | 52 | (11.1%) | 91 (9.2%) |
| Constipation | 39 | (7.5%) | 16 | (3.4%) | 55 (5.5%) |

### Summary of TEAE Topics of Special Interest (CMQ Search)—FAS

| | Previously | | Rollover Population | | | |
|---|---|---|---|---|---|---|
| | Unexposed | | Previously Treated | | | |
| Safety Topics of | Population | | with Tavapadon | | Overall | |
| Special Interest | (N = 523) | | (N = 468) | | (N = 991) | |
| (CMQ Search) | n (%) | | n (%) | | n (%) | |
| Nausea | 95 | (18.2%) | 23 | (4.9%) | 118 | (11.9%) |
| Headache | 78 | (14.9%) | 22 | (4.7%) | 100 | (10.1%) |
| Fall | 57 | (10.9%) | 34 | (7.3%) | 91 | (9.2%) |
| Hallucination | 43 | (8.2%) | 19 | (4.1%) | 62 | (6.3%) |
| Somnolence | 26 | (5.0%) | 24 | (5.1%) | 50 | (5.0%) |
| Hypotension | 27 | (5.2%) | 14 | (3.0%) | 41 | (4.1%) |
| Orthostatic | 19 | (3.6%) | 18 | (3.8%) | 37 | (3.7%) |
| Hypotension | | | | | | |
| REM Sleep | 25 | (4.8%) | 12 | (2.6%) | 37 | (3.7%) |
| Behavior Disorders | | | | | | |
| Abuse Potential | 8 | (1.5%) | 7 | (1.5%) | 15 | (1.5%) |
| Impulse Control | 10 | (1.9%) | 4 | (0.9%) | 14 | (1.4%) |
| Disorders | | | | | | |

| | | | | | |
|---|---|---|---|---|---|
| Suicidality | 0 | 3 | (0.6%) | 3 | (0.3%) |

### Efficacy

The efficacy of tavapadon in both early and advanced PD populations was maintained during long-term treatment with tavapadon. There were no signals or trends suggesting loss of efficacy with long-term treatment.

The key efficacy endpoints below were assessed and summarized using descriptive statistics.
a. Change from baseline in the MDS-UPDRS Parts I, II, and III scores
b. Change from baseline in the Hauser diary (only in subjects who require daily L-Dopa or equivalent therapy for symptom control at the time of enrollment)

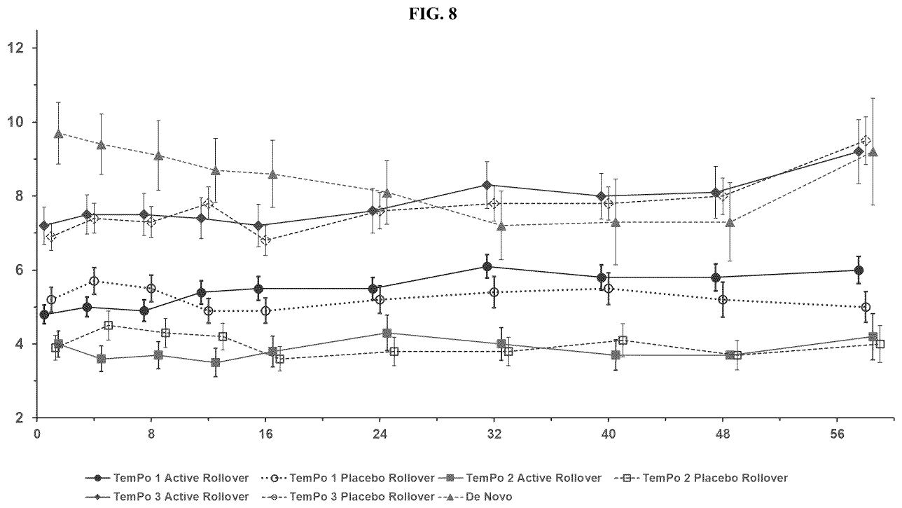

MDS UPDRS Part I (Non-Motor Aspects of Experiences of Daily Living) Scores (FIG. 8)
a. MDS-UPDRS Part I consists of 13 items and has a score range 0-52 with higher score indicating more impairment.
b. MDS-UPDRS Part 1 score in early PD populations (PD-001 and PD-002 rollover) was generally stable over the OLE exposure; no unexpected findings.
c. In the advanced populations (PD-003 rollover, De Novo) the Part I score was higher versus early PD population.
d. The score in PD-003 rollover population was stable up to Week 16 with a slight upward trend afterwards.
e. A reduction we observed in the de novo population with an upward trend after Week 48.

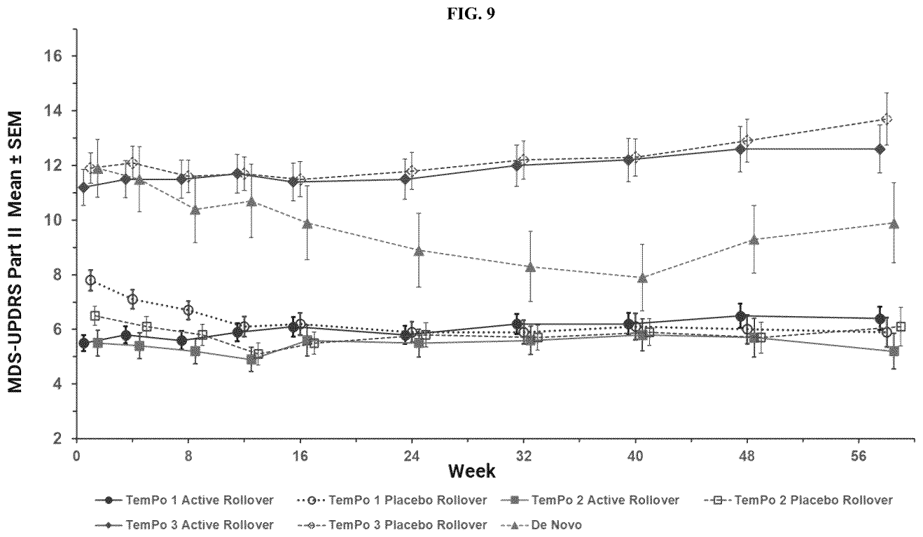

MDS UPDRS Parts II (Motor Aspects of Experiences of Daily Living) Scores (FIG. 9)
a. MDS-UPDRS Part II consists of 13 items and has a score range 0-52 with higher score indicating more impairment.
b. In previously treated early PD populations (PD-001 and PD-002 active rollover), the baseline score was lower reflecting the reduction observed in the DB trials. The scores was stable over the OLE exposure with no trend of deterioration observed.
c. In previously unexposed early PD populations (PD-001 and PD-002 placebo rollover), the baseline score was higher than previously treated groups with an improvement observed in the first 12 weeks and then remained stable over the OLE exposure with no trend of deterioration observed.
d. In the advanced populations (PD-003 rollover, De Novo) the Part II score was higher versus early PD population.
e. An apparent slight upward trend was observed after 24 weeks of OLE treatment in PD-003 rollover population. The de novo population had a reduction up to Week 40 and a slight upward trend was observed afterwards.

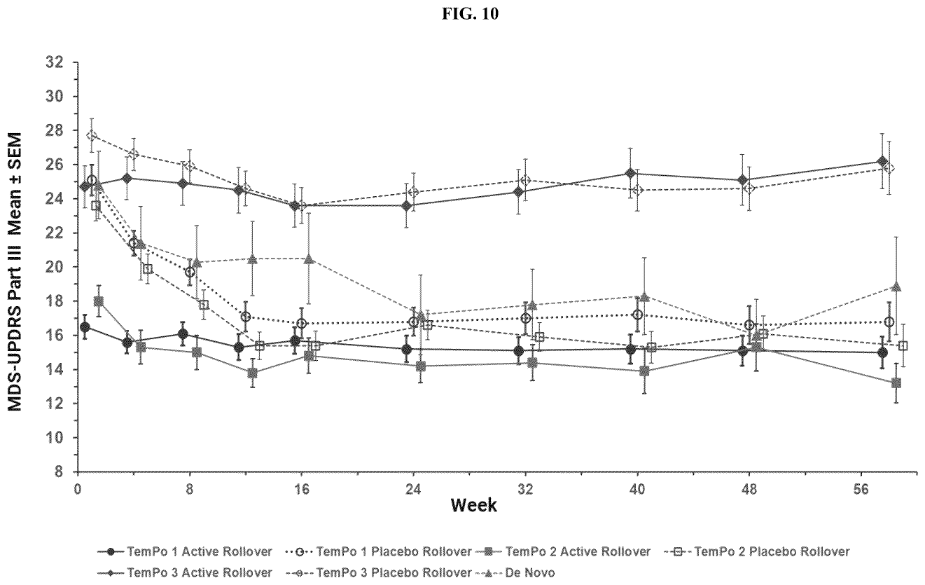

MDS UPDRS Parts III (Motor Examination) Scores (FIG. 10)
a. MDS-UPDRS Part III comprises 18 items resulting in 33 scores by location/lateralization and has a score range 0-132 with higher score indicating more impairment.
b. In previously treated early PD populations (PD-001 and PD-002 active rollover), the baseline score was lower reflecting the reduction observed in the DB trials. The scores were stable over the OLE exposure with no trend of deterioration observed.
c. In previously unexposed early PD populations (PD-001 and PD-002 placebo rollover), the baseline score was higher than previously treated groups with an improvement observed in the first 16 weeks and then remained stable over the OLE exposure with no trend of deterioration observed.
d. In the previously treated advanced population (PD-003 active rollover) the Part III score was stable up to Week 24 with a slight upward trend afterwards.
e. In the previously unexposed advanced population (PD-003 placebo rollover) a reduction in Part III score was observed up to Week 16 with a slight upward trend afterwards.
f. A reduction was observed in the de novo population up to Week 24 with the score remaining stable afterwards.

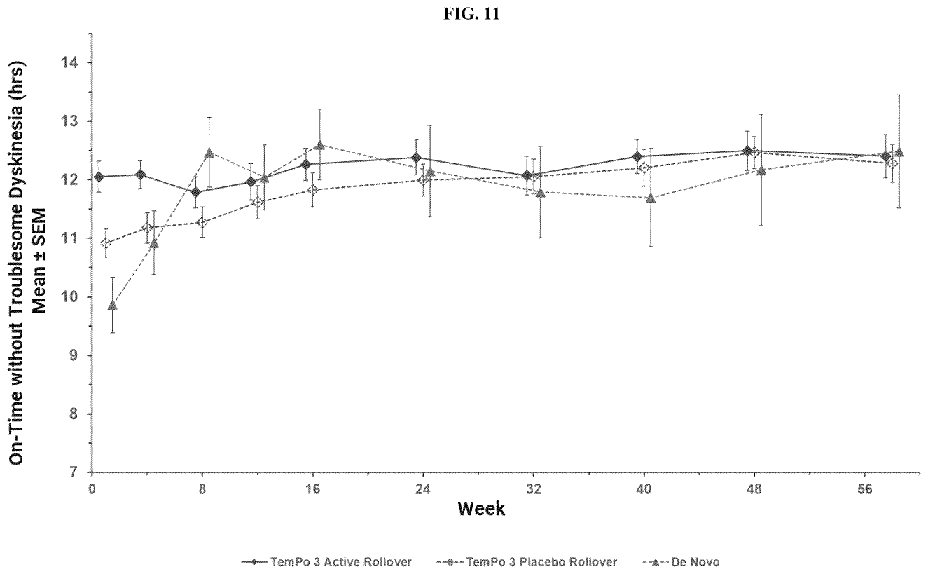

On-time without Troublesome Dyskinesia in Patients on Daily L-Dopa at the Time of Enrollment (FIG. 11)
a. In previously treated advanced PD population (PD-003 active rollover), the on-time without troublesome dyskinesia was stable throughout with no trend of deterioration.
b. In previously unexposed advanced populations (PD-003 placebo rollover and de novo), an increase in on-time without troublesome dyskinesia was observed.

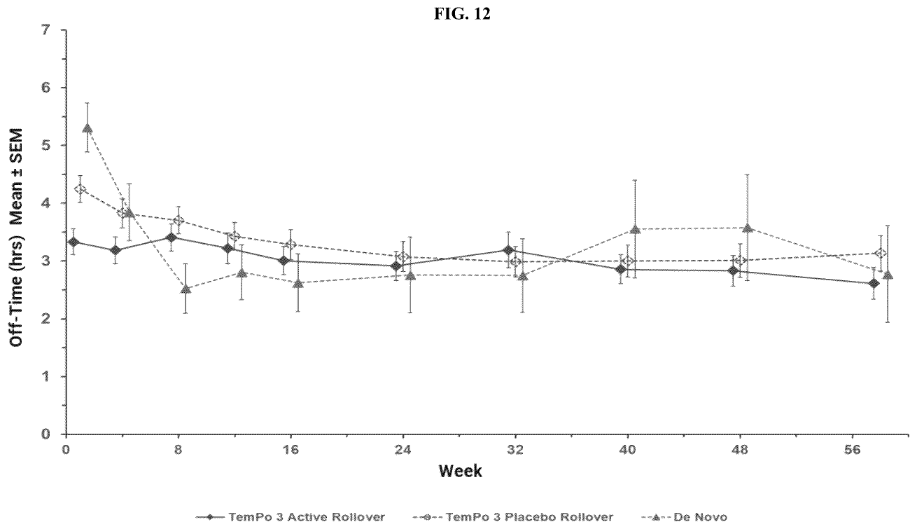

Off-time in Patients on Daily L-Dopa at the Time of Enrollment (FIG. 12)
a. In previously treated advanced PD population (PD-003 active rollover), the off time was stable throughout with no trend of deterioration.
b. In previously unexposed advanced populations (PD-003 placebo rollover and de novo), a decrease in off time on tavapadon treatment was observed.

All patents and publications mentioned in this specification are incorporated herein by reference in their entireties. From the foregoing description, it will be apparent that variations and modifications can be made to the invention described herein to adopt it to various uses and conditions. Such embodiments are also within the scope of the following claims.

1. A method for treating Parkinson's Disease in a patient, wherein the method comprises:
a titration phase comprising orally administering to the patient a daily dose of tavapadon in eight sequential dose escalation steps 1-8, wherein
step 2 comprises administering to the patient a dose of 0.5 mg per day of tavapadon on days 5-8 of treatment,
step 6 comprises administering to the patient a dose of 2.25 mg per day of tavapadon on days 21-24 of treatment,
step 8 comprises administering to the patient a dose of a therapeutically effective amount of 5 mg per day of tavapadon,
thereby treating the Parkinson's Disease.

2. (canceled)

3. (canceled)

4. (canceled)

5. The method of claim 1, wherein the method further comprises an adjustment phase following the titration phase, wherein the adjustment phase comprises:
step 9 comprising administering to the patient a dose of 10 mg per day dose of tavapadon, and
step 10 comprising administering to the patient a therapeutically effective amount of 15 mg per day dose of tavapadon;
wherein the dose of tavapadon is adjusted over the range of a therapeutically effective amount of 5, 10 or 15 mg per day of tavapadon in step 9 and step 10 based on patient tolerability.

6. The method of claim 1, wherein the method further comprises an adjustment phase following the titration phase, wherein the adjustment phase comprises:
step 9 comprising administering to the patient a dose of 10 mg per day dose of tavapadon, wherein a patient who is unable to increase the dose from 5 mg to 10 mg on the first day of step 9 due to tolerability issues will begin a maintenance phase at the patient's maximum tolerated dose of 5 mg per day.

7. The method of claim 5, wherein the method further comprises a maintenance phase following the adjustment phase, wherein the maintenance phase comprises administering to the patient a therapeutically effective amount of 5, 10 or 15 mg per day dose of tavapadon, wherein the patient is administered the highest tolerated dose achieved during the adjustment phase.

8. The method of claim 7, wherein the dose administered to the patient in the maintenance phase is a maximum tolerated dose of tavapadon.

9. The method of claim 7, wherein tavapadon is administered to the patient in a dose of 3 or fewer oral pills.

10-21. (canceled)

22. The method of claim 1, wherein the patient has a diagnosis of Parkinson's Disease that is consistent with the UK Parkinson's Disease Society Brain Bank diagnostic criteria.

23. The method of claim 1, wherein the patient has modified Hoehn and Yahr stage 1, 1.5, or 2.

24. The method of claim 1, wherein the patient has modified Hoehn and Yahr stage 2, 2.5, or 3 in the “on” state and a minimum of 22 hours of “off” time on 2 consecutive days.

25. The method of claim 1, wherein the patient has a Unified Parkinson's Disease Rating Scale (MDS-UPDRS) Part II score≥2 and Part III score≥10.

26. The method of claim 1, wherein the patient is not previously treated with a medication for Parkinson's Disease.

27. The method of claim 1, wherein the patient is previously and/or concurrently treated with a medication for Parkinson's Disease.

28. The method of claim 7, wherein the patient is previously treated with a dopaminergic agent.

29. The method of claim 7, wherein the patient is concurrently treated with a dopaminergic agent.

30. The method of claim 29, wherein the dopaminergic agent is levodopa or dopamine receptor agonist.

31-35. (canceled)

36. The method of claim 1, wherein the method comprises administering to the patient one or more catechol-O-methyl transferase inhibitor, MAO-B inhibitor, amantadine, istradefylline, or anticholinergic drug prior to or concurrently with tavapadon.

37. (canceled)

38. The method of claim 1, wherein the Parkinson's Disease is an early Parkinson's Disease.

39. The method of claim 7, wherein the Parkinson's Disease is an early Parkinson's Disease.

40. The method of claim 39, wherein the treatment results in a statistically significant and clinical meaningful improvement over placebo at Week 26 from commencement of the treatment as measured by a Movement Disorder Society-Unified Parkinson's Disease Rating Scale (MDS-UPDRS) Parts II+III Combined Score.

41. The method of claim 39, wherein the combined score in the patient is improved by −12.1, wherein the patient is administered a therapeutically effective amount of 15 mg per day dose of tavapadon in the maintenance phase.

42. The method of claim 39, wherein the combined score in the patient is improved by −11.5, wherein the patient is administered a therapeutically effective amount of 5 mg per day dose of tavapadon in the maintenance phase.

43. The method of claim 39, wherein the treatment results in a statistically significant and clinical meaningful improvement over placebo at Week 26 from commencement of the treatment as measured by a Movement Disorder Society-Unified Parkinson's Disease Rating Scale (MDS-UPDRS) Part II Score.

44. (canceled)

45. (canceled)

46. The method of claim 1, wherein the Parkinson's Disease is an advanced Parkinson's Disease.

47. The method of claim 7, wherein the Parkinson's Disease is an advanced Parkinson's Disease.

48. (canceled)

49. The method of claim 1, wherein the treatment results in an increased daily “on” time by about 1.1 hours without troublesome dyskinesia over placebo at Week 26 from commencement of the treatment.

50. The method of claim 1, wherein the treatment results in a reduced daily “off” time by about 0.95 hours without troublesome dyskinesia over placebo at Week 26 from commencement of the treatment.

51. The method of claim 1, wherein the patient exhibits improved motor control symptoms or improved motor function, as measured on the Hoehn and Yahr scale.

52. (canceled)

53. (canceled)

54. A method for treating Parkinson's Disease in a patient, wherein the method comprises:
a titration phase comprising orally administering to the patient:
0.25 mg per day dose of tavapadon for days 1-4 of the titration phase;
0.5 mg per day dose of tavapadon for days 5-8 of the titration phase;
0.75 mg per day dose of tavapadon for days 9-12 of the titration phase;
1.0 mg per day dose of tavapadon for days 13-16 of the titration phase;
1.5 mg per day dose of tavapadon for days 17-20 of the titration phase;
2.25 mg per day dose of tavapadon for days 21-24 of the titration phase;
3 mg per day dose of tavapadon for days 25-40 of the titration phase; and
a therapeutically effective amount of 5 mg per day dose of tavapadon for days 41-56 of the titration phase;
thereby treating the Parkinson's Disease.

55. A method for treating Parkinson's Disease in a patient, wherein the method comprises:
i. a titration phase comprising orally administering to the patient:
0.25 mg per day dose of tavapadon for days 1-4 of the titration phase;
0.5 mg per day dose of tavapadon for days 5-8 of the titration phase;
0.75 mg per day dose of tavapadon for days 9-12 of the titration phase;
1.0 mg per day dose of tavapadon for days 13-16 of the titration phase;
1.5 mg per day dose of tavapadon for days 17-20 of the titration phase; 2.25 mg per day dose of tavapadon for days 21-24 of the titration phase;
3 mg per day dose of tavapadon for days 25-40 of the titration phase; and a therapeutically effective amount of 5 mg per day dose of tavapadon for days 41-56 of the titration phase;
ii. followed by an adjustment phase comprising orally administering to the patient:
a therapeutically effective amount of 10 mg per day dose of tavapadon, and
a therapeutically effective amount of 15 mg per day dose of tavapadon,
wherein the dose of tavapadon is adjusted over the range of 5, 10 or 15 mg based on patient tolerability; and
iii. followed by a maintenance phase comprising orally administering to the patient:
a therapeutically effective amount of 5, 10 or 15 mg per day dose of tavapadon;
wherein the patient is administered the highest tolerated dose achieved during the adjustment phase and the maintenance phase maintains the patient on a steady dose of tavapadon,
thereby treating the Parkinson's Disease.

56. A method for treating Parkinson's Disease in a patient, wherein the method comprises:
i. a titration phase comprising orally administering to the patient:
0.25 mg per day dose of tavapadon for days 1-4 of the titration phase;
0.5 mg per day dose of tavapadon for days 5-8 of the titration phase;
0.75 mg per day dose of tavapadon for days 9-12 of the titration phase;
1.0 mg per day dose of tavapadon for days 13-16 of the titration phase;
1.5 mg per day dose of tavapadon for days 17-20 of the titration phase; 2.25 mg per day dose of tavapadon for days 21-24 of the titration phase;
3 mg per day dose of tavapadon for days 25-40 of the titration phase; and a therapeutically effective amount of 5 mg per day dose of tavapadon for days 41-56 of the titration phase;
ii. followed by an adjustment phase comprising orally administering to the patient: a therapeutically effective amount of 10 mg per day dose of tavapadon, and
wherein a patient who is unable to increase the dose from 5 mg to 10 mg on the first day of step 9 due to tolerability issues will begin a maintenance phase at the patient's maximum tolerated dose of 5 mg per day; and
iii. followed by a maintenance phase comprising orally administering to the patient:
a therapeutically effective amount of 5, 10 or 15 mg per day dose of tavapadon;
wherein the patient is administered the highest tolerated dose achieved during the adjustment phase and the maintenance phase maintains the patient on a steady dose of tavapadon,
thereby treating the Parkinson's Disease.

57-72. (canceled)
